SEQUENCE LISTING (6 sequences)

SEQ ID NO: 1 - protein, 357 aa, Artificial Sequence [sequence omitted]

SEQ ID NO: 2 - protein, 357 aa, Artificial Sequence
  Synthetic polypeptide
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 131 aa, Artificial Sequence
  Synthetic polypeptide
PTSSSTKKTQLQLEHLLLDLQMILNGINNYKNPKLTRMLTFKFYMPKKATELKHLQLEEELKPLEEVLNLAQSKNFHLRPRDLISNINVIVLELKGSETTFMCEYADETATIVEFLNRWITFSQSIISTLT

SEQ ID NO: 4 - protein, 188 aa, Artificial Sequence
  Synthetic polypeptide
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 5 - protein, 133 aa, Artificial Sequence [sequence omitted]

SEQ ID NO: 6 - protein, 132 aa, Artificial Sequence
  Synthetic polypeptide
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

(19) United States
(12) Patent Application Publication
(10) Pub. No.: US 2024/0148827 A1
(43) Pub. Date: May 9, 2024

(54) Methods and Compositions for Treatment of Disease

(71) Applicants: The Methodist Hospital, Houston, TX (US); Coya Therapeutics, Inc., Houston, TX (US)

(72) Inventors: Stanley Hersh Appel, Houston, TX (US); Alireza Faridar, Houston, TX (US); Howard Berman, Houston, TX (US)

(21) Appl. No.: 18/280,632

(22) PCT Filed: Mar. 10, 2022
(86) PCT No.: PCT/US2022/019748

§ 371 Date: Sep. 6, 2023

Related U.S. Application Data
(60) Provisional application No. 63/310,839, filed on Feb. 16, 2022.
(60) Provisional application No. 63/225,846, filed on Jul. 26, 2021.
(60) Provisional application No. 63/159,919, filed on Mar. 11, 2021.

(51) Int. Cl. A61K 38/17; A61K 9/00; A61K 38/20; A61K 45/06; A61P 25/28

(52) U.S. Cl. CPC A61K 38/1774; A61K 9/0019; A61K 38/2013; A61K 45/06; A61P 25/28

(57) ABSTRACT

The present disclosure provides methods for treating diseases such as neurodegenerative and neuroinflammatory diseases, for example, Alzheimer's disease, comprising administration of a CTLA-4-containing protein, e.g., abatacept, and an IL-2 protein, e.g., aldesleukin, to a subject, either separately or in a single formulation. Also presented herein are pharmaceutical compositions comprising a CTLA-4-containing protein, e.g., abatacept, and an IL-2 protein, e.g., aldesleukin.

### CROSS-REFERENCE TO RELATED APPLICATIONS

This application claims the benefit of U.S. Provisional Application No. 63/159,919 filed Mar. 11, 2021, U.S. Provisional Application No. 63/225,846 filed Jul. 26, 2021, and U.S. Provisional Application No. 63/310,839 filed Feb. 16, 2022, each of which is incorporated by reference herein in its entirety.

### REFERENCE TO SEQUENCE LISTING SUBMITTED ELECTRONICALLY

This application incorporates by reference a Sequence Listing submitted with this application as text file entitled “14678-014-228_SEQ_LISTING.txt,” created on Mar. 9, 2022, and having 12,142 bytes in size.

### 1. FIELD

The present disclosure provides methods for treating diseases such as neurodegenerative and neuroinflammatory diseases, for example, Alzheimer's disease, comprising administration of a CTLA-4-containing protein, e.g., abatacept, and an IL-2 protein, e.g., aldesleukin, to a subject, either separately or in a single formulation. Also presented herein are pharmaceutical compositions comprising a CTLA-4-containing protein, e.g., abatacept, and an IL-2 protein, e.g., aldesleukin.

### 2. BACKGROUND

Inflammatory and neuroinflammatory mechanisms contribute to a wide variety of devastating diseases, including such neurodegenerative diseases as amyotrophic lateral sclerosis (ALS), Alzheimer's disease and Parkinson's disease. Neurodegenerative diseases such as this direct a tremendous health and economic burden that will only exacerbate further over time.

Currently, no disease-modifying treatments for such diseases are available. Anti-inflammatory treatments have been utilized for decades in attempting to ameliorate a multitude of neurodegenerative diseases. Little progress, however, has been made with single drug/target approaches.

Increasingly, studies point to immune system involvement in the etiology of diseases such as this, and point to dysfunction of immune cells as a chief mediator of disease pathogenesis. The complex signaling mechanisms and built-in redundancies of the immune system and its constituents may help explain the ineffectiveness of such single drug/single target anti-inflammatory approaches.

Recently great promise has been demonstrated with regulatory T cell (Treg) cell therapy, which may represent a more global approach to suppressing immune system dysfunction contributing to disease. For example, clinical trials involving administration of expanded autologous Tregs to ALS patients report that the Treg therapy slowed progression rates during early and later stages of the disease, and that Treg suppressive function correlated with the slowing of disease progression (Thonhoff, J. R. et al., 2018, Neurology-Neuroimmunology Neuroinflammation 5(4)).

Nonetheless, there still exists a need for development of additional treatments that can suppress inflammatory and/or promote anti-inflammatory immune system components.

### 3. SUMMARY

In one aspect, presented herein is a method of treating a neurodegenerative or neuroinflammatory disease or disorder in a subject in need thereof, comprising administering to the subject:
i) a CTLA-4-containing protein; and
ii) an IL-2 protein;
wherein the method mitigates one or more symptoms associated with the neurodegenerative or neuroinflammatory disease or disorder in the treated subject. In certain embodiments, the CTLA-4-containing protein is abatacept. In certain embodiments, the IL-2 protein is aldesleukin. In certain embodiments, the CTLA-4-containing protein is abatacept and the IL-2 protein is aldesleukin.

In certain embodiments, the CTLA-4-containing protein is administered by injection or infusion. In particular embodiments, the CTLA-4-containing protein is administered subcutaneously. In particular embodiments, the CTLA-4-containing protein is administered intravenously. In certain embodiments, the IL-2 protein is administered by injection or infusion. In particular embodiments, the IL-2 protein is administered subcutaneously. In particular embodiments, the IL-2 protein is administered intravenously. In certain embodiments, the CTLA-4-containing protein and the IL-2 protein are administered by injection or infusion. In particular embodiments, the CTLA-4-containing protein and the IL-2 protein are administered subcutaneously. In particular embodiments, the CTLA-4-containing protein and the IL-2 protein are administered intravenously.

In certain embodiments, the CTLA-4-containing protein comprises a human CTLA-4 extracellular domain. In particular embodiments, the CTLA-4-containing protein is a fusion protein, for example, a fusion protein that comprises a human CTLA-4 extracellular domain and a human immunoglobulin Fe domain, e.g., a modified Fe domain that comprises an immunoglobulin hinge region, CH2 region and CH3. In particular embodiments, the human immunoglobulin Fc domain is a human IgG1 Fc domain. In certain embodiments, the CTLA-4-containing protein is glycosylated.

In certain embodiments, the CTLA-4-containing protein comprises the following amino acid sequence monomer: MHVAQPAVVLASSRGIASFVCEYASPGKATEVRVTVLRQADSQVTEVCAATYMMG NELTFLDDSICTGTSSGNQVNLTIQGLRAMDTGLYICKVELMYPPPYYLGIGNGTQIY VIDPEPCPDSDQEPKSSDKTHTSPPSPAPELLGGSSVFLFPPKPKDTLMISRTPEVTCVV VDVSHEDPEVKFNWYVDGVEVHNAKTKPREEQYNSTYRVVSVLTVLHQDWLNGK EYKCKVSNKALPAPIEKTISKAKGQPREPQVYTLPPSRDELTKNQVSLTCLVKGFYPS DIAVEWESNGQPENNYKTTPPVLDSDGSFFLYSKLTVDKSRWQQGNVFSCSVMHEA LHNHYTQKSLSLSPGK (SEQ ID NO:1). In specific embodiments, the CTLA-4-containing protein comprises a homodimer of two monomers, each monomer comprising the amino acid sequence of SEQ ID NO:1.

In particular embodiments, the CTLA-4-containing protein is abatacept.

In certain embodiments, the CTLA-4-containing protein comprises the following amino acid sequence monomer:

MHVAQPAVVLASSRGIASFVCEYASPGKYTEVRVTVLRQADSQVTEVCA ATYMMGNELTFLDDSICTGTSSGNQVNLTIQGLRAMDTGLYICKVELMYPPPYYEGI GNGTQIYVIDPEPCPDSDQEPKSSDKTHTSPPSPAPELLGGSSVFLFPPKPKDTLMISRT PEVTCVVVDVSHEDPEVKFNWYVDGVEVHNAKTKPREEQYNSTYRVVSVLTVLHQ DWLNGKEYKCKVSNKALPAPIEKTISKAKGQPREPQVYTLPPSRDELTKNQVSLTCL VKGFYPSDIAVEWESNGQPENNYKTTPPVLDSDGSFFLYSKLTVDKSRWQQGNVFS CSVMHEALHNHYTQKSLSLSPGK (SEQ ID NO:2). In specific embodiments, the CTLA-4-containing protein comprises a homodimer of two monomers, each monomer comprising the amino acid sequence of SEQ ID NO:2.

In particular embodiments, the CTLA-4-containing protein is belatacept.

In certain embodiments, the IL-2 protein is a human IL-2 protein. In particular embodiments, the human IL-2 protein comprises a serine at the amino acid position corresponding to native mature human IL-2 amino acid residue 125. In certain embodiments, the human IL-2 protein lacks an N-terminal alanine amino acid. In particular embodiments, the human IL-2 protein lacks an N-terminal alanine amino acid and comprises a serine at the amino acid position corresponding to native mature human IL-2 amino acid residue 125.

In certain embodiments, the human IL-2 protein comprises the following amino acid sequence:

(SEQ ID NO: 3)
PTSSSTKKTQLQLEHLLLDLQMILNGINNYKNPKLTRMLTFKFYMPKKA
TELKHLQLEEELKPLEEVLNLAQSKNFHLRPRDLISNINVIVLELKGSE
TTFMCEYADETATIVEFLNRWITFSQSIISTLT.

In specific embodiments, the IL-2 protein is not glycosylated. In certain embodiments, the IL-2 protein is aldesleukin.

In certain embodiments, the human IL-2 protein comprises the following amino acid sequence:

(SEQ ID NO: 6)
PTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGS
ETTFMCEYADETATIVEFLNRWITFSQSIISTLT.

In one aspect, presented herein is a method of treating a neurodegenerative or neuroinflammatory disease or disorder in a subject in need thereof, comprising administering to the subject:
i) abatacept; and
ii) aldesleukin;
wherein the method mitigates one or more symptoms associated with the neurodegenerative or neuroinflammatory disease or disorder in the treated subject.

In certain embodiments, the abatacept is administered by injection or infusion. In particular embodiments, the abatacept is administered subcutaneously. In particular embodiments, the abatacept is administered intravenously. In certain embodiments, the aldesleukin is administered by injection or infusion. In particular embodiments, the aldesleukin is administered subcutaneously. In particular embodiments, the aldesleukin is administered intravenously. In certain embodiments, the abatacept and the aldesleukin are administered by injection or infusion. In particular embodiments, the abatacept and the aldesleukin are administered subcutaneously. In particular embodiments, the abatacept and the aldesleukin are administered intravenously.

In certain embodiments, the CTLA-4-containing protein, e.g., abatacept, is administered to the subject over the course of 2 months, 3 months, 4 months, 5 months, 6 months, 1 year, 2 years, 5 years, 10 years or more.

In certain embodiments, the CTLA-4-containing protein, e.g., abatacept, is administered to the subject once every two weeks. In particular embodiments, the CTLA-4-containing protein, e.g., abatacept, is administered to the subject subcutaneously once every two weeks. In certain embodiments, the CTLA-4-containing protein, e.g., abatacept, is administered to the subject once every two weeks for 15 weeks, sixteen weeks, 4 months, 5 months, 6 months, 1 year, 2 years, 5 years, 10 years or more. In particular embodiments, the CTLA-4-containing protein, e.g., abatacept, is administered to the subject subcutaneously once every two weeks for 15 weeks, sixteen weeks, 4 months, 5 months, 6 months, 1 year, 2 years, 5 years, 10 years or more.

In certain embodiments, the CTLA-4-containing protein, e.g., abatacept, is administered to the subject once every three weeks. In particular embodiments, the CTLA-4-containing protein, e.g., abatacept, is administered to the subject subcutaneously once every three weeks. In certain embodiments, the CTLA-4-containing protein, e.g., abatacept, is administered to the subject once every three weeks for 15 weeks, sixteen weeks, 4 months, 5 months, 6 months, 1 year, 2 years, 5 years, 10 years or more. In particular embodiments, the CTLA-4-containing protein, e.g., abatacept, is administered to the subject subcutaneously once every three weeks for 15 weeks, sixteen weeks, 4 months, 5 months, 6 months, 1 year, 2 years, 5 years, 10 years or more.

In certain embodiments, the CTLA-4-containing protein, e.g., abatacept, is administered to the subject once every four weeks. In particular embodiments, the CTLA-4-containing protein, e.g., abatacept, is administered to the subject subcutaneously once every four weeks. In certain embodiments, the CTLA-4-containing protein, e.g., abatacept, is administered to the subject once every four weeks for 15 weeks, sixteen weeks, 4 months, 5 months, 6 months, 1 year, 2 years, 5 years, 10 years or more. In particular embodiments, the CTLA-4-containing protein, e.g., abatacept, is administered to the subject subcutaneously once every four weeks for 15 weeks, sixteen weeks, 4 months, 5 months, 6 months, 1 year, 2 years, 5 years, 10 years or more.

In certain embodiments, the IL-2 protein, e.g., aldesleukin, is administered to the subject once daily for three consecutive days. In particular embodiments, the IL-2 protein, e.g., aldesleukin, is administered to the subject subcutaneously once daily for three consecutive days.

In certain embodiments, the CTLA-4-containing protein, e.g., abatacept, is administered to the subject once every two weeks and the IL-2 protein, e.g., aldesleukin, is administered to the subject once daily for three consecutive days. In certain embodiments, the CTLA-4-containing protein, e.g., abatacept, is administered to the subject subcutaneously once every two weeks and the IL-2 protein, e.g., aldesleukin, is administered to the subject subcutaneously once daily for three consecutive days.

In certain embodiments, the CTLA-4-containing protein, e.g., abatacept, is administered to the subject once every two weeks and the IL-2 protein, e.g., aldesleukin, is administered to the subject once daily for three consecutive days beginning on the day the CTLA-4-containing protein, e.g., abatacept, is administered. In certain embodiments, the CTLA-4-containing protein, e.g., abatacept, is administered to the subject subcutaneously once every two weeks and the IL-2 protein, e.g., aldesleukin, is administered to the subject subcutaneously once daily for three consecutive days, beginning on the day the CTLA-4-containing protein, e.g., abatacept, is administered to the subject.

In certain embodiments, the CTLA-4-containing protein, e.g., abatacept, is administered to the subject once every two weeks for fifteen weeks, sixteen weeks, 4 months, 5 months, 6 months, 1 year, 2 years, 5 years, 10 years or more, and the IL-2 protein is administered to the subject once daily for three consecutive days beginning on the day the CTLA-4-containing protein, e.g., abatacept, is administered to the subject. In particular embodiments, the first administration of the CTLA-4-containing protein to the subject is performed in the absence of an IL-2 protein administration. For example, in certain embodiments, the IL-2 protein, e.g., aldesleukin, administration to the subject begins on week three and the IL-2 protein is administered to the subject daily for three consecutive days beginning on the day the CTLA-4-containing protein, e.g., abatacept, is administered to the subject. In certain embodiments, the CTLA-4-containing protein, e.g., abatacept, is administered to the subject subcutaneously once every two weeks for fifteen weeks, sixteen weeks, 4 months, 5 months, 6 months, 1 year, 2 years, 5 years, 10 years or more, and the IL-2 protein is administered to the subject subcutaneously once daily for three consecutive days beginning on the day the CTLA-4-containing protein, e.g., abatacept, is administered. In particular embodiments, the first administration of the CTLA-4-containing protein to the subject is performed in the absence of an IL-2 protein administration. For example, in certain embodiments, the IL-2 protein, e.g., aldesleukin, administration to the subject begins on week three and the IL-2 protein is subcutaneously administered to the subject daily for three consecutive days beginning on the day the CTLA-4-containing protein, e.g., abatacept, is administered to the subject.

In certain embodiments, the CTLA-4-containing protein, e.g., abatacept, is administered to the subject once every three weeks and the IL-2 protein, e.g., aldesleukin, is administered to the subject once daily for three consecutive days. In certain embodiments, the CTLA-4-containing protein, e.g., abatacept, is administered to the subject subcutaneously once every three weeks and the IL-2 protein, e.g., aldesleukin, is administered to the subject subcutaneously once daily for three consecutive days.

In certain embodiments, the CTLA-4-containing protein, e.g., abatacept, is administered to the subject once every three weeks and the IL-2 protein, e.g., aldesleukin, is administered to the subject once daily for three consecutive days beginning on the day the CTLA-4-containing protein, e.g., abatacept, is administered. In certain embodiments, the CTLA-4-containing protein, e.g., abatacept, is administered to the subject subcutaneously once every three weeks and the IL-2 protein, e.g., aldesleukin, is administered to the subject subcutaneously once daily for three consecutive days, beginning on the day the CTLA-4-containing protein, e.g., abatacept, is administered to the subject.

In certain embodiments, the CTLA-4-containing protein, e.g., abatacept, is administered to the subject once every three weeks for fifteen weeks, sixteen weeks, 4 months, 5 months, 6 months, 1 year, 2 years, 5 years, 10 years or more, and the IL-2 protein is administered to the subject once daily for three consecutive days beginning on the day the CTLA-4-containing protein, e.g., abatacept, is administered to the subject. In certain embodiments, the CTLA-4-containing protein, e.g., abatacept, is administered to the subject subcutaneously once every three weeks for fifteen weeks, sixteen weeks, 4 months, 5 months, 6 months, 1 year, 2 years, 5 years, 10 years or more, and the IL-2 protein is administered to the subject subcutaneously once daily for three consecutive days beginning on the day the CTLA-4-containing protein, e.g., abatacept, is administered. In particular embodiments, the first administration of the CTLA-4-containing protein to the subject is performed in the absence of an IL-2 protein administration.

In certain embodiments, the CTLA-4-containing protein, e.g., abatacept, is administered to the subject once every three weeks and the IL-2 protein, e.g., aldesleukin, is administered to the subject once daily for three consecutive days beginning on the day the CTLA-4-containing protein, e.g., abatacept, is administered. In certain embodiments, the CTLA-4-containing protein, e.g., abatacept, is administered to the subject subcutaneously once every three weeks and the IL-2 protein, e.g., aldesleukin, is administered to the subject subcutaneously once daily for three consecutive days, beginning on the day the CTLA-4-containing protein, e.g., abatacept, is administered to the subject.

In certain embodiments, the CTLA-4-containing protein, e.g., abatacept, is administered to the subject once every four weeks for fifteen weeks, sixteen weeks, 4 months, 5 months, 6 months, 1 year, 2 years, 5 years, 10 years or more, and the IL-2 protein is administered to the subject once daily for three consecutive days beginning on the day the CTLA-4-containing protein, e.g., abatacept, is administered to the subject. In certain embodiments, the CTLA-4-containing protein, e.g., abatacept, is administered to the subject subcutaneously once every four weeks for fifteen weeks, sixteen weeks, 4 months, 5 months, 6 months, 1 year, 2 years, 5 years, 10 years or more, and the IL-2 protein is administered to the subject subcutaneously once daily for three consecutive days beginning on the day the CTLA-4-containing protein, e.g., abatacept, is administered. In particular embodiments, the first administration of the CTLA-4-containing protein to the subject is performed in the absence of an IL-2 protein administration.

In certain embodiments, about 5 mg to about 200 mg, about 10 mg to about 200 mg, about 15 mg to about 200 mg, about 20 mg to about 200 mg, about 25 mg to about 200 mg, about 50 to about 200 mg, about 50 mg to about 175 mg, about 50 mg to about 150 mg or about 50 mg to about 125 mg of the CTLA-4-containing protein, e.g., abatacept, is administered to the subject. In a particular embodiment, the CTLA-4-containing protein, e.g., abatacept, is subcutaneously administered to the subject.

In a particular embodiment, about 50 mg of the CTLA-4-containing protein, e.g., abatacept, is administered to the subject. In a specific embodiment, about 50 mg of the CTLA-4-containing protein, e.g., abatacept, in a 0.4 mL volume is administered to the subject. In a particular embodiment, the CTLA-4-containing protein, e.g., abatacept, is subcutaneously administered to the subject.

In a particular embodiment, about 87.5 mg of the CTLA-4-containing protein, e.g., abatacept, is administered to the subject. In a specific embodiment, about 87.5 mg of the CTLA-4-containing protein, e.g., abatacept, in a 0.7 mL volume is administered to the subject. In a particular embodiment, the CTLA-4-containing protein, e.g., abatacept, is subcutaneously administered to the subject.

In a particular embodiment, about 125 mg of the CTLA-4-containing protein, e.g., abatacept, is administered to the subject. In a specific embodiment, about 125 mg of the CTLA-4-containing protein, e.g., abatacept, in a 1.0 mL volume is administered to the subject. In a particular embodiment, the CTLA-4-containing protein, e.g., abatacept, is subcutaneously administered to the subject.

In a particular embodiment, about 1×10⁴ to about 1×10⁷, about 5×10⁴ to about 1×10⁷, about 1×10⁵ to about 1×10⁷, about 5×10⁵ to about 1×10⁷, 5×10⁵ to about 5×10⁶, 5×10⁵ to about 4×10⁶, 5×10⁵ to about 3×10⁶, 5×10⁵ to about 2×10⁶, about 5×10⁵ to about 1×10⁶ units IL-2 protein, e.g., aldesleukin, is administered to the subject. In a particular embodiment, the IL-2 protein, e.g., aldesleukin, is subcutaneously administered to the subject.

In a specific embodiment, about 500,000 units to 3,000,000 units of the IL-2 protein, e.g., aldesleukin, is administered to the subject, for example, is subcutaneously administered to the subject. In a specific embodiment, about 500,000 units to 2,000,000 units of the IL-2 protein, e.g., aldesleukin, is administered to the subject, for example, is subcutaneously administered to the subject. In a specific embodiment, about 500,000 units to 1,000,000 units of the IL-2 protein, e.g., aldesleukin, is administered to the subject, for example, is subcutaneously administered to the subject.

In one aspect, presented herein is a method of treating a neurodegenerative or neuroinflammatory disease or disorder in a subject in need thereof, wherein the method comprises administering to the subject a formulation comprising:
i) a CTLA-4-containing protein; and
ii) an IL-2 protein;
wherein the method mitigates one or more symptoms associated with the neurodegenerative or neuroinflammatory disease or disorder in the treated subject. The formulation is administered to the subject one or more times. In certain embodiments, the formulation is administered to the subject over the course of 2 months, 3 months, 4 months, 5 months, 6 months, 1 year, 2 years, 5 years, 10 years or more. In certain embodiments, the CTLA-4-containing protein is abatacept. In certain embodiments, the CTLA-4-containing protein is belatacept. In certain embodiments, the IL-2 protein is aldesleukin. In certain embodiments, the CTLA-4-containing protein is abatacept and the IL-2 protein is aldesleukin.

In one aspect, presented herein is a method of treating a neurodegenerative or neuroinflammatory disease or disorder in a subject in need thereof, comprising a dosing cycle that begins on day 1 and comprises administering to the subject a formulation comprising:
i) a CTLA-4-containing protein; and
ii) an IL-2 protein; wherein the method mitigates one or more symptoms associated with the neurodegenerative or neuroinflammatory disease or disorder in the treated subject. The formulation may be administered to the subject one or more times during the dosing cycle.

The method may comprise one or more dosing cycles. In certain embodiments, the method comprises more than one dosing cycle and each dosing cycle is the same. In certain embodiments, the method comprises more than one dosing cycle and at least one dosing cycle differs from another. A dosing cycle may be repeated one or more times. There may be a period of time between the completion of one dosing cycle and the beginning of the next dosing cycle.

In certain embodiments, the CTLA-4-containing protein is abatacept. In certain embodiments, the CTLA-4-containing protein is belatacept. In certain embodiments, the IL-2 protein is aldesleukin. In certain embodiments, the CTLA-4-containing protein is abatacept and the IL-2 protein is aldesleukin.

For ease of description, the formulation may be referred to herein as a “CTLA-4-containing protein/IL-2 protein formulation,” or an “IL-2 protein/CTLA-4-containing protein formulation.” In instances wherein the CTLA-4-containing protein is abatacept and the IL-2 protein is aldesleukin, the formulation may be referred to herein as an “abatacept/aldesleukin formulation” or an “aldesleukin/abatacept formulation.”

In certain embodiments, the CTLA-4-containing protein/IL-2 protein formulation, e.g., the abatacept/aldesleukin formulation, is administered to the subject by injection or infusion. In specific embodiments, the CTLA-4-containing protein/IL-2 protein formulation, e.g., the abatacept/aldesleukin formulation, is administered to the subject subcutaneously. In specific embodiments, the CTLA-4-containing protein/IL-2 protein formulation, e.g., the abatacept/aldesleukin formulation, is administered to the subject intravenously.

In certain embodiments of such a method, the dosing cycle comprises administering the CTLA-4-containing protein/IL-2 protein formulation, e.g., the abatacept/aldesleukin formulation, to the subject 1-10 times.

In specific embodiments, of the method described herein, the dosing cycle comprises a single administration of the CTLA-4-containing protein/IL-2 protein formulation, e.g., the abatacept/aldesleukin formulation, to the subject on day 1 of the dosing cycle. In specific embodiments of such a method, the dosing cycle comprises administering the CTLA-4-containing protein/IL-2 protein formulation, e.g., the abatacept/aldesleukin formulation, to the subject daily for two consecutive days, beginning on day 1 of the dosing cycle. In specific embodiments of such a method, the dosing cycle comprises administering the CTLA-4-containing protein/IL-2 protein formulation, e.g., the abatacept/aldesleukin formulation, to the subject daily for three consecutive days, beginning on day 1 of the dosing cycle. In specific embodiments of such a method, the dosing cycle comprises administering the CTLA-4-containing protein/IL-2 protein formulation, e.g., the abatacept/aldesleukin formulation, to the subject daily for four consecutive days, beginning on day 1 of the dosing cycle. In specific embodiments of such a method, the dosing cycle comprises administering the CTLA-4-containing protein/IL-2 protein formulation, e.g., the abatacept/aldesleukin formulation, to the subject daily for five consecutive days, beginning on day 1 of the dosing cycle. In specific embodiments of such a method, the dosing cycle comprises administering the CTLA-4-containing protein/IL-2 protein formulation, e.g., the abatacept/aldesleukin formulation, to the subject daily for six consecutive days, beginning on day 1 of the dosing cycle. In specific embodiments of such a method, the dosing cycle comprises administering the CTLA-4-containing protein/IL-2 protein formulation, e.g., the abatacept/aldesleukin formulation, to the subject daily for seven consecutive days, beginning on day 1 of the dosing cycle.

In specific embodiments of the methods described herein, the dosing cycle comprises administering the CTLA-4-containing protein/IL-2 protein formulation, e.g., the abatacept/aldesleukin formulation, to the subject daily for at least two non-consecutive days. In one non-limiting embodiment, for example, the CTLA-4-containing protein/IL-2 protein formulation, e.g., the abatacept/aldesleukin formulation, is first administered to the subject on day 1 and is next administered to the subject on day 3, day 4, day 5, day 6 or day 7 of the dosing cycle.

In certain embodiments of the methods described herein, a method comprises 2-13 dosing cycles. In particular embodiments of the method described herein, the dosing cycle is repeated 1-12 times. In specific embodiments, a method comprises 7 dosing cycles, e.g., the dosing cycle is repeated 6 times. In particular embodiments, each dosing cycle, e.g., each repeated dosing cycle, begins 10-28 days after day 1 of the previous dosing cycle. In particular embodiments, each dosing cycle, e.g., each repeated dosing cycle, begins 10-28 days after the completion of the previous dosing cycle. In specific embodiments, each dosing cycle, e.g., each repeated dosing cycle, begins 14 days after day 1 of the previous dosing cycle. In particular embodiments, each dosing cycle, e.g., each repeated dosing cycle, begins 14 days after the completion of the previous dosing cycle.

In certain embodiments of the methods described herein, the first dosing cycle comprises administering the CTLA-4-containing protein/IL-2 protein formulation, e.g., the abatacept/aldesleukin formulation, to the subject daily for three consecutive days, beginning on day 1 of the dosing cycle, and the first dosing cycle is repeated 6 times, with each repeated dosing cycle beginning 14 days after day 1 of the previous dosing cycle.

In certain embodiments of the methods described herein, the dosing cycle comprises administering to the subject a CTLA-4-containing protein/IL-2 protein formulation comprising about 5 mg to about 125 mg CTLA-4-containing protein and about 3×10⁴ to about 3×10⁷ units IL-2 protein. In certain embodiments of the methods described herein, the dosing cycle comprises administering to the subject an abatacept/aldesleukin formulation comprising about 5 mg to about 125 mg abatacept and about 3×10⁴ to about 3×10⁷ units aldesleukin.

In certain embodiments of the methods described herein, the dosing cycle comprises administering to the subject a formulation comprising about 8.75 mg to about 87.5 mg abatacept and about 3×10⁴ to about 3×10⁷ units aldesleukin.

In certain embodiments of the methods described herein, the dosing cycle comprises administering to the subject a formulation comprising about 29.17 mg abatacept and about 1×10⁵ units aldesleukin. In certain embodiments of the methods described herein, the dosing cycle comprises administering to the subject a formulation comprising about 29.17 mg abatacept and about 1×10⁶ units aldesleukin. In certain embodiments of the methods described herein, the dosing cycle comprises administering to the subject a formulation comprising about 29.17 mg abatacept and about 1×10⁷ units aldesleukin.

In certain embodiments of the methods described herein, the dosing cycle comprises administering to the subject a formulation comprising about 5 mg to about 50 mg abatacept and about 3×10⁴ to about 3×10⁷ units aldesleukin.

In certain embodiments of the methods described herein, the dosing cycle comprises administering to the subject a formulation comprising about 16.67 mg abatacept and about 1×10⁵ units aldesleukin. In certain embodiments of the methods described herein, the dosing cycle comprises administering to the subject a formulation comprising about 16.67 mg abatacept and about 1×10⁶ units aldesleukin. In certain embodiments of the methods described herein, the dosing cycle comprises administering to the subject a formulation comprising about 16.67 mg abatacept and about 1×10⁷ units aldesleukin.

In certain embodiments of the methods described herein, the dosing cycle comprises administering to the subject a formulation comprising about 12.5 mg to about 125 mg abatacept and about 3×10⁴ to about 3×10⁷ units aldesleukin.

In certain embodiments of the methods described herein, the dosing cycle comprises administering to the subject a formulation comprising about 41.67 mg abatacept and about 1×10⁵ units aldesleukin. In certain embodiments of the methods described herein, the dosing cycle comprises administering to the subject a formulation comprising about 41.67 mg abatacept and about 1×10⁶ units aldesleukin. In certain embodiments of the methods described herein, the dosing cycle comprises administering to the subject a formulation comprising about 41.67 mg abatacept and about 1×10⁷ units aldesleukin.

In certain embodiments of the methods described herein, the dosing cycle comprises administering the formulation to the subject daily for three consecutive days, beginning on day 1 of the dosing cycle, wherein the formulation comprises about 29.17 mg abatacept and about 1×10⁶ units aldesleukin. In specific embodiments, the dosing cycle is repeated 6 times, with each repeated dosing cycle beginning 14 days after day 1 of the previous dosing cycle.

In certain of the embodiments of the methods described herein, dosing cycles continue over the course of 2 months, 3 months, 4 months, 5 months, 6 months, 1 year, 2 years, 5 years, 10 years or more, e.g., dosing cycles are repeated over the course of 2 months, 3 months, 4 months, 5 months, 6 months, 1 year, 2 years, 5 years, 10 years or more.

In certain embodiments, the methods described herein further comprise administering a CTLA-4-containing protein formulation, e.g., an abatacept formulation, to the subject prior to the first administration to the subject of the CTLA-4-containing/IL-2 protein formulation, e.g., the abatacept/aldesleukin formulation. In certain embodiments, the methods described herein further comprise administering a CTLA-4-containing protein formulation, e.g., an abatacept formulation, to the subject 14 days prior to day 1 of the first dosing cycle, that is, 14 days prior to the first administration to the subject of the CTLA-4-containing/IL-2 protein formulation, e.g., the abatacept/aldesleukin formulation.

In specific embodiments, the CTLA-4-containing protein formulation comprises 50 mg to 125 mg CTLA-4-containing protein, for example, 50 mg CTLA-4-containing protein, 87.5 mg CTLA-4-containing protein or 125 mg CTLA-4-containing protein. In specific embodiments, the abatacept formulation comprises 50 mg to 125 mg abatacept, for example, 50 mg abatacept, 87.5 mg abatacept or 125 mg abatacept. In certain embodiments, the CTLA-4-containing protein formulation, e.g., abatacept formulation, is administered to the subject by injection or infusion. In certain embodiments, the CTLA-4-containing protein formulation, e.g., abatacept formulation, is administered to the subject subcutaneously or intravenously.

In certain embodiments of the methods described herein, the neurodegenerative disease or disorder is amyotrophic lateral sclerosis, Alzheimer's disease, Parkinson's disease, multiple sclerosis, frontotemporal dementia or Huntington's disease. In certain embodiments of the methods described herein, the neurodegenerative disease or disorder is Alzheimer's disease.

In certain embodiments of the methods described herein, the neuroinflammatory disease or disorder is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

In certain embodiments of the methods described herein, the method further comprises performing an additional therapeutic intervention, wherein the additional therapeutic intervention comprises a cognitive rehabilitation program, a neurostimulation technique, or a combination thereof. In certain embodiments, the cognitive rehabilitation program is a computer-implemented cognitive rehabilitation program. In certain embodiments, the neurostimulation technique is an invasive brain stimulation (IBS) technique. In some embodiments, the neurostimulation technique is a non-invasive brain stimulation (NIBS) technique. In some embodiments, the IBS technique is selected from the group consisting of: deep brain stimulation (DBS) and invasive vagus nerve stimulation (VNS). In some embodiments, the NIBS technique is selected from the group consisting of transcranial magnetic stimulation (TMS), transcranial direct current stimulation (tDCS), transcranial alternating current stimulation (tACS), electroconvulsive treatment (ECT), magnetic seizure therapy (MST), cranial electrostimulation (CES), and non-invasive VNS.

In one aspect, presented herein is a kit, comprising, in separate containers, i) one or more doses of a formulation comprising 50 to 125 mg abatacept, and ii) one or more doses of a formulation comprising 500,000 to 3,000,000 units aldesleukin. In certain embodiments, the kit comprises one or more doses of a formulation comprising 50 mg abatacept, 87.5 mg abatacept or 125 mg abatacept. In specific embodiments, the one or more doses of abatacept are present in lyophilized form, e.g., are present as a lyophilized powder or cake. In certain embodiments, the formulation of one or more doses of abatacept is suitable for subcutaneous administration or intravenous administration. In certain embodiments, the kit comprises one or more doses of a formulation comprising 500,000 to 2,000,000 units aldesleukin or 1,000,000 units aldesleukin. In specific embodiments, the one or more doses of aldesleukin are present in lyophilized form, e.g., are present as a lyophilized powder or cake. In certain embodiments, the formulation of one or more doses of aldesleukin is suitable for subcutaneous administration or intravenous administration.

In one aspect, presented herein is a pharmaceutical composition comprising one or more doses of a CTLA-4-containing protein and an IL-2 protein (“CTLA-4-containing protein/IL-2 protein doses”). In certain embodiments, presented herein is a pharmaceutical composition comprising one or more doses of abatacept and aldesleukin (“abatacept/aldesleukin doses”).

In certain embodiments, presented herein is a pharmaceutical composition comprising one or more abatacept/aldesleukin doses, wherein an abatacept/aldesleukin dose comprises 5 mg to 125 mg abatacept and 3×10⁴ to 3×10⁷ units aldesleukin.

In particular embodiments, presented herein is a pharmaceutical composition comprising one or more abatacept/aldesleukin doses, wherein an abatacept/aldesleukin dose comprises 8.75 to 87.5 mg abatacept and 3×10⁴ to 3×10⁷ units aldesleukin. In specific embodiments, presented herein is a pharmaceutical composition comprising one or more abatacept/aldesleukin doses, wherein an abatacept/aldesleukin dose comprises 29.17 mg abatacept and 1×10⁵ units aldesleukin, 1×10⁶ units aldesleukin or 1×10⁷ units aldesleukin. In specific embodiments, presented herein is a pharmaceutical composition comprising one or more abatacept/aldesleukin doses, wherein an abatacept/aldesleukin dose comprises 29.17 mg abatacept and 1×10⁶ units aldesleukin.

In particular embodiments, presented herein is a pharmaceutical composition comprising one or more abatacept/aldesleukin doses, wherein an abatacept/aldesleukin dose comprises 5 mg to 50 mg abatacept and 3×10⁴ to 3×10⁷ units aldesleukin. In specific embodiments, presented herein is a pharmaceutical composition comprising one or more abatacept/aldesleukin doses, wherein an abatacept/aldesleukin dose comprises 16.67 mg abatacept and 1×10⁵ units aldesleukin, 1×10⁶ units aldesleukin or 1×10⁷ units aldesleukin. In specific embodiments, presented herein is a pharmaceutical composition comprising one or more abatacept/aldesleukin doses, wherein an abatacept/aldesleukin dose comprises 16.67 mg abatacept and 1×10⁶ units aldesleukin.

In particular embodiments, presented herein is a pharmaceutical composition comprising one or more abatacept/aldesleukin doses, wherein an abatacept/aldesleukin dose comprises 12.5 mg to 125 mg abatacept and 3×10⁴ to 3×10⁷ units aldesleukin. In specific embodiments, presented herein is a pharmaceutical composition comprising one or more abatacept/aldesleukin doses, wherein an abatacept/aldesleukin dose comprises 41.67 mg abatacept and 1×10⁵ units aldesleukin, 1×10⁶ units aldesleukin or 1×10⁷ units aldesleukin. In specific embodiments, presented herein is a pharmaceutical composition comprising one or more abatacept/aldesleukin doses, wherein an abatacept/aldesleukin dose comprises 41.67 mg abatacept and 1×10⁶ units aldesleukin.

In certain embodiments, presented herein is a pharmaceutical composition comprising one or more abatacept/aldesleukin doses as shown in Table 4.

In certain embodiments, a pharmaceutical composition comprising one or more abatacept/aldesleukin doses described herein is present in lyophilized form, for example, is present as a lyophilized powder or lyophilized cake.

In certain embodiments, a pharmaceutical composition comprising one or more abatacept/aldesleukin doses described herein is a solution, for example, an aqueous solution. In specific embodiments, the one or more abatacept/aldesleukin doses are present in the pharmaceutical composition at a concentration of 1 abatacept/aldesleukin dose/0.4 ml, 1 abatacept/aldesleukin dose/0.7 ml, 1 abatacept/aldesleukin dose/1.0 ml, 1 abatacept/aldesleukin dose/1.5 ml or 1 abatacept/aldesleukin dose/2.0 ml.

In certain embodiments, a pharmaceutical composition comprising one or more abatacept/aldesleukin doses described herein is suitable for subcutaneous administration. In certain embodiments, a pharmaceutical composition comprising one or more abatacept/aldesleukin doses described herein is suitable for intravenous administration.

Further illustrative embodiments are as follows:
1. A method of treating a neurodegenerative or neuroinflammatory disease or disorder in a subject in need thereof, comprising administering to the subject:
i) a CTLA-4-containing protein; and
ii) an IL-2 protein;
wherein the method mitigates one or more symptoms associated with the neurodegenerative or neuroinflammatory disease or disorder in the treated subject.
2. The method of embodiment 1, wherein the CTLA-4-containing protein comprises a human CTLA-4 extracellular domain.
3. The method of embodiment 1 or 2, wherein the CTLA-4-containing protein is a fusion protein.
4. The method of embodiment 3, wherein the fusion protein comprises a human CTLA-4 extracellular domain and a human immunoglobulin Fc domain.
5. The method of embodiment 4, wherein the Fe domain is a modified Fe domain that comprises an immunoglobulin hinge region, CH2 region and CH3.
6. The method of embodiment 4 or 5, wherein the human immunoglobulin Fe domain is a human IgG1 Fe domain.
7. The method of any one of embodiments 1-6, wherein the CTLA-4-containing protein is glycosylated.
8. The method of any one of embodiments 1-7, wherein the CTLA-4-containing protein the following amino acid sequence monomer:

(SEQ ID NO: 1)
MHVAQPAVVLASSRGIASFVCEYASPGKATEVRVTVLRQADSQVTEVCA
ATYMMGNELTFLDDSICTGTSSGNQVNLTIQGLRAMDTGLYICKVELMY
PPPYYLGIGNGTQIYVIDPEPCPDSDQEPKSSDKTHTSPPSPAPELLGG
SSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGK.

9. The method of embodiment 8, wherein the CTLA-4-containing protein comprises a homodimer of two monomers, each comprising the amino acid sequence of SEQ ID NO:1.
10. The method of embodiment 1, wherein the CTLA-4-containing protein is abatacept.
11. The method of any one of embodiments 1-10, wherein the IL-2 protein is a human IL-2 protein.
12. The method of embodiment 11, wherein the human IL-2 protein comprises a serine at the amino acid position corresponding to native mature human IL-2 amino acid residue 125.
13. The method of embodiment 11 or 12, wherein the human IL-2 protein lacks an N-terminal alanine amino acid.
14. The method of any one of embodiments 11-13, wherein the human IL-2 protein comprises the following amino acid sequence:

(SEQ ID NO: 3)
PTSSSTKKTQLQLEHLLLDLQMILNGINNYKNPKLTRMLTFKFYMPKKA
TELKHLQLEEELKPLEEVLNLAQSKNFHLRPRDLISNINVIVLELKGSE
TTFMCEYADETATIVEFLNRWITFSQSIISTLT.

15. The method of any one of embodiments 1-14, wherein the IL-2 protein is not glycosylated.
16. The method of embodiment 11, wherein the IL-2 protein is aldesleukin.
17. A method of treating a neurodegenerative or neuroinflammatory disease or disorder in a subject in need thereof, comprising administering to the subject:
i) abatacept; and
ii) aldesleukin;
wherein the method mitigates one or more symptoms associated with the neurodegenerative or neuroinflammatory disease or disorder in the treated subject.
18. The method of embodiment 17, wherein the abatacept is administered by injection or infusion.
19. The method of 18, wherein the abatacept is administered subcutaneously.
20. The method of 18, wherein the abatacept is administered intravenously.
21. The method of embodiment 17, wherein the aldesleukin is administered by injection or infusion.
22. The method of 21, wherein the aldesleukin is administered subcutaneously.
23. The method of 21, wherein the aldesleukin is administered intravenously.
24. The method of embodiment 17, wherein the abatacept and the aldesleukin are administered subcutaneously.
25. The method of embodiment 17, wherein the abatacept and the aldesleukin are administered intravenously.
26. The method of any one of embodiments 17-25, wherein the abatacept is administered once every two weeks.
27. The method of embodiment 26, wherein the abatacept is administered subcutaneously once every two weeks.
28. The method of embodiment 26 or 27, wherein the abatacept is administered once every two weeks for 15 weeks.
29. The method of any one of embodiments 17-28, wherein the aldesleukin is administered once daily for three consecutive days.
30. The method of embodiment 29, wherein the aldesleukin is administered subcutaneously once daily for three consecutive days.
31. The method of any one of embodiments 17-25, wherein:
a) the abatacept is administered once every two weeks; and
b) the aldesleukin is administered once daily for three consecutive days beginning on the day the abatacept is administered.
32. The method of embodiment 31, wherein the abatacept and the aldesleukin are administered subcutaneously.
33. The method of any one of embodiments 17-25, wherein:
a) the abatacept is administered once every two weeks for fifteen weeks;
b) aldesleukin administration begins on week three; and
c) once aldesleukin administration begins, the aldesleukin is administered once daily for three consecutive days beginning on the day the abatacept is administered.
34. The method of embodiment 33, wherein the abatacept and the aldesleukin are administered subcutaneously.
35. The method of any one of embodiments 17-34, wherein the abatacept is administered in an amount in the range of 50 mg to 125 mg.
36. The method of embodiment 35, wherein the abatacept is administered in a 50 mg amount.
37. The method of embodiment 36, wherein the abatacept is subcutaneously administered in a 0.4 mL volume.
38. The method of embodiment 35, wherein the abatacept is administered in an 87.5 mg amount.
39. The method of embodiment 38, wherein the abatacept is subcutaneously administered in a 0.7 mL volume.
40. The method of embodiment 35, wherein the abatacept is administered in a 125 mg amount.
41. The method of embodiment 40, wherein the abatacept is subcutaneously administered in a 1.0 mL volume.
42. The method of any one of embodiments 17-41, wherein the aldesleukin is administered in an amount in the range of 500,000 units to 3,000,000 units.
43. The method of embodiment 42, wherein the aldesleukin is administered in an amount in the range of 500,000 units to 2,000,000 units.
44. The method of embodiment 43, wherein the aldesleukin is administered in an amount of 1,000,000 units.
45. The method of any one of embodiments 42-44, wherein the aldesleukin is administered subcutaneously.
46. A method of treating a neurodegenerative or neuroinflammatory disease or disorder in a subject in need thereof, comprising a dosing cycle that begins on day 1 and comprises administering to the subject a formulation comprising:
i) abatacept; and
ii) aldesleukin;
wherein the method mitigates one or more symptoms associated with the neurodegenerative or neuroinflammatory disease or disorder in the treated subject.
47. The method of embodiment 46, wherein the formulation is administered by injection or infusion.
48. The method of embodiment 46, wherein the formulation is administered subcutaneously.
49. The method of embodiment 46, wherein the formulation is administered intravenously.
50. The method of any one of embodiments 46-49, wherein the dosing cycle comprises administering the formulation to the subject 1-10 times.
51. The method of any one of embodiments 46-49, wherein the dosing cycle comprises a single administration of the formulation to the subject on day 1 of the dosing cycle.
52. The method of any one of embodiments 46-49, wherein the dosing cycle comprises administering the formulation to the subject daily for two consecutive days, beginning on day 1 of the dosing cycle.
53. The method of any one of embodiments 46-49, wherein the dosing cycle comprises administering the formulation to the subject daily for three consecutive days, beginning on day 1 of the dosing cycle.
54. The method of any one of embodiments 46-49, wherein the dosing cycle comprises administering the formulation to the subject daily for four consecutive days, beginning on day 1 of the dosing cycle.
55. The method of any one of embodiments 46-49, wherein the dosing cycle comprises administering the formulation to the subject daily for five consecutive days, beginning on day 1 of the dosing cycle.
56. The method of any one of embodiments 46-55, wherein the dosing cycle is repeated 1-12 times.
57. The method of any one of embodiments 46-55, wherein the dosing cycle is repeated 6 times.
58. The method of embodiment 56 or 57, wherein each repeated dosing cycle begins 10-28 days after day 1 of the previous dosing cycle.
59. The method of any one of embodiments 56-58, wherein each repeated dosing cycle begins 14 days after day 1 of the previous dosing cycle.
60. The method of any one of embodiments 46-49, wherein the first dosing cycle comprises administering the formulation to the subject daily for three consecutive days, beginning on day 1 of the dosing cycle, and the first dosing cycle is repeated 6 times, with each repeated dosing cycle beginning 14 days after day 1 of the previous dosing cycle.
61. The method of any one of embodiments 46-49, wherein the dosing cycle comprises administering to the subject a formulation comprising about 5 mg to about 125 mg abatacept and about 3×10⁴ to about 3×10⁷ units aldesleukin.
62. The method of any one of embodiments 46-49, wherein the dosing cycle comprises administering to the subject a formulation comprising about 8.75 mg to about 87.5 mg abatacept and about 3×10⁴ to about 3×10⁷ units aldesleukin.
63. The method of any one of embodiments 46-49, wherein the dosing cycle comprises administering to the subject a formulation comprising about 29.17 mg abatacept and about 1×10⁵ units aldesleukin.
64. The method of any one of embodiments 46-49, wherein the dosing cycle comprises administering to the subject a formulation comprising about 29.17 mg abatacept and about 1×10⁶ units aldesleukin.
65. The method of any one of embodiments 46-49, wherein the dosing cycle comprises administering to the subject a formulation comprising about 29.17 mg abatacept and about 1×10⁷ units aldesleukin.
66. The method of any one of embodiments 46-49, wherein the dosing cycle comprises administering to the subject a formulation comprising about 5 mg to about 50 mg abatacept and about 3×10⁴ to about 3×10⁷ units aldesleukin.
67. The method of any one of embodiments 46-49, wherein the dosing cycle comprises administering to the subject a formulation comprising about 16.67 mg abatacept and about 1×10⁵ units aldesleukin.
68. The method of any one of embodiments 46-49, wherein the dosing cycle comprises administering to the subject a formulation comprising about 16.67 mg abatacept and about 1×10⁶ units aldesleukin.
69. The method of any one of embodiments 46-49, wherein the dosing cycle comprises administering to the subject a formulation comprising about 16.67 mg abatacept and about 1×10⁷ units aldesleukin.
70. The method of any one of embodiments 46-49, wherein the dosing cycle comprises administering to the subject a formulation comprising about 12.5 mg to about 125 mg abatacept and about 3×10⁴ to about 3×10⁷ units aldesleukin.
71. The method of any one of embodiments 46-49, wherein the dosing cycle comprises administering to the subject a formulation comprising about 41.67 mg abatacept and about 1×10⁵ units aldesleukin.
72. The method of any one of embodiments 46-49, wherein the dosing cycle comprises administering to the subject a formulation comprising about 41.67 mg abatacept and about 1×10⁶ units aldesleukin.
73. The method of any one of embodiments 46-49, wherein the dosing cycle comprises administering to the subject a formulation comprising about 41.67 mg abatacept and about 1×10⁷ units aldesleukin.
74. The method of any one of embodiments 46-49, wherein the dosing cycle comprises administering the formulation to the subject daily for three consecutive days, beginning on day 1 of the dosing cycle, wherein the formulation comprises about 29.17 mg abatacept and about 1×10⁶ units aldesleukin.
75. The method of embodiment 74, wherein the dosing cycle is repeated 6 times, with each repeated dosing cycle beginning 14 days after day 1 of the previous dosing cycle.
76. The method of embodiment 75, wherein the dosing cycle comprises 1-10 administrations of an abatacept/aldesleukin formulation as shown at Table 1A.
77. The method of any one of embodiments 46-49, wherein a total of 50 mg abatacept and 3×10⁶ units aldesleukin are administered to the subject per dosing cycle.
78. The method of embodiment 77, wherein the dosing cycle comprises 1-10 administrations of an abatacept/aldesleukin formulation as shown at Table 1B.
79. The method of any one of embodiments 46-49, wherein a total of 50 mg abatacept and 3×10⁷ units aldesleukin are administered to the subject per dosing cycle.
80. The method of embodiment 79, wherein the dosing cycle comprises 1-10 administrations of an abatacept/aldesleukin formulation as shown at Table 1C.
81. The method of any one of embodiments 46-49, wherein a total of 87.5 mg abatacept and 3×10⁵ units aldesleukin are administered to the subject per dosing cycle.
82. The method of embodiment 81, wherein the dosing cycle comprises 1-10 administrations of an abatacept/aldesleukin formulation as shown at Table 2A.
83. The method of any one of embodiments 46-49, wherein a total of 87.5 mg abatacept and 3×10⁶ units aldesleukin are administered to the subject per dosing cycle.
84. The method of embodiment 83, wherein the dosing cycle comprises 1-10 administrations of an abatacept/aldesleukin formulation as shown at Table 2B.
85. The method of any one of embodiments 46-49, wherein a total of 87.5 mg abatacept and 3×10⁷ units aldesleukin are administered to the subject per dosing cycle.
86. The method of embodiment 85, wherein the dosing cycle comprises 1-10 administrations of an abatacept/aldesleukin formulation as shown at Table 2C.
87. The method of any one of embodiments 46-49, wherein a total of 125 mg abatacept and 3×10⁵ units aldesleukin are administered to the subject per dosing cycle.
88. The method of embodiment 87, wherein the dosing cycle comprises 1-10 administrations of an abatacept/aldesleukin formulation as shown at Table 3A.
89. The method of any one of embodiments 46-49, wherein a total of 125 mg abatacept and 3×10⁶ units aldesleukin are administered to the subject per dosing cycle.
90. The method of embodiment 89, wherein the dosing cycle comprises 1-10 administrations of an abatacept/aldesleukin formulation as shown at Table 3B.
91. The method of any one of embodiments 46-49, wherein a total of 125 mg abatacept and 3×10⁷ units aldesleukin are administered to the subject per dosing cycle.
92. The method of embodiment 91, wherein the dosing cycle comprises 1-10 administrations of an abatacept/aldesleukin formulation as shown at Table 3C.
93. The method of any one of embodiments 46-92, wherein the dosing cycle is repeated 6 times, with each repeated dosing cycle beginning 14 days after day 1 of the previous dosing cycle.
94. The method of any one of embodiments 46-92, wherein the formulation is administered by injection or infusion.
95. The method of any one of embodiments 46-92, wherein the formulation is administered subcutaneously.
96. The method of any one of embodiments 46-92, wherein the formulation is administered intravenously.
97. The method of any one of embodiments 46-96, further comprising administering an abatacept formulation to the subject 14 days prior to day 1 of the first dosing cycle, wherein the abatacept formulation comprises abatacept.
98. The method of embodiment 97, wherein the abatacept formulation comprises 50 mg to 125 mg abatacept.
99. The method of embodiment 97, wherein the abatacept formulation comprises 87.5 mg abatacept.
100. The method of any one of embodiments 97-99, wherein the abatacept formulation is administered by injection or infusion.
101. The method of any one of embodiments 97-99, wherein the abatacept formulation is administered subcutaneously.
102. The method of any one of embodiments 97-99, wherein the abatacept formulation is administered intravenously.
103. The method of any one of embodiments 97-99, wherein the neurodegenerative disease or disorder is amyotrophic lateral sclerosis, Alzheimer's disease, Parkinson's disease, multiple sclerosis, frontotemporal dementia or Huntington's disease.
104. The method of embodiment 103, wherein the neurodegenerative disease or disorder is Alzheimer's disease.
105. The method of any one of embodiments 1-104, wherein the neuroinflammatory disease or disorder is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.
106. The method of any one of embodiments 1-105, wherein the method further comprises performing a cognitive rehabilitation program, a neurostimulation technique, or a combination thereof.
107. The method of embodiment 106, wherein the cognitive rehabilitation program is a computer-implemented cognitive rehabilitation program.
108. The method of embodiment 105 or 106, wherein the neurostimulation technique is an invasive brain stimulation (IBS) technique.
109. The method of embodiment 105 or 106, wherein the neurostimulation technique is a non-invasive brain stimulation (NIBS) technique.
110. The method of embodiment 108, wherein the IBS technique is selected from the group consisting of: deep brain stimulation (DBS) and invasive vagus nerve stimulation (VNS).
111. The method of embodiment 109, wherein the NIBS technique is selected from the group consisting of transcranial magnetic stimulation (TMS), transcranial direct current stimulation (tDCS), transcranial alternating current stimulation (tACS), electroconvulsive treatment (ECT), magnetic seizure therapy (MST), cranial electrostimulation (CES), and non-invasive VNS.
112. A kit, comprising, in separate containers, i) one or more doses of a formulation comprising 50 to 125 mg abatacept, and ii) one or more doses of a formulation comprising 500,000 to 3,000,000 units aldesleukin.
113. The kit of embodiment 112, wherein the kit comprises one or more doses of a formulation comprising 50 mg abatacept.
114. The kit of embodiment 112, wherein the kit comprises one or more doses of a formulation comprising 87.5 mg abatacept.
115. The kit of embodiment 112, wherein the kit comprises one or more doses of a formulation comprising 125 mg abatacept.
116. The kit of any one of embodiments 112-115, wherein the kit comprises one or more doses of a formulation of 500,000 to 2,000,000 units aldesleukin.
117. The kit of any one of embodiments 112-116, wherein the kit comprises one or more doses of a formulation of 1,000,000 units aldesleukin.
118. The kit of any one of embodiments 112-117, wherein the one or more doses of abatacept are present in lyophilized form.
119. The kit of embodiment 118, wherein the one or more doses of abatacept are present as a lyophilized powder or lyophilized cake.
120. The kit of any one of embodiments 112-119, wherein the one or more doses of aldesleukin are present in lyophilized form.
121. The kit of embodiment 120, wherein the one or more doses of aldesleukin are present as a lyophilized powder or lyophilized cake.
122. The kit of any one of embodiments 112-121, wherein the formulation of one or more doses of abatacept is suitable for subcutaneous administration.
123. The kit of any one of embodiments 112-121, wherein the formulation of one or more doses of abatacept is suitable for intravenous administration.
124. The kit of any one of embodiments 112-123, wherein the formulation of one or more doses of aldesleukin is suitable for subcutaneous administration.
125. The kit of any one of embodiments 112-123, wherein the formulation of one or more doses of aldesleukin is suitable for intravenous administration.
126. A pharmaceutical composition comprising one or more abatacept/aldesleukin doses.
127. The pharmaceutical composition of embodiment 126, wherein an abatacept/aldesleukin dose comprises 5 mg to 125 mg abatacept and 3×10⁴ to 3×10⁷ units aldesleukin.
128. The pharmaceutical composition of embodiment 126, wherein an abatacept/aldesleukin dose comprises 8.75 mg to about 87.5 mg abatacept and 3×10⁴ to 3×10⁷ units aldesleukin.
129. The pharmaceutical composition of embodiment 126, wherein an abatacept/aldesleukin dose comprises about 29.17 mg abatacept and about 1×10⁵ units aldesleukin.
130. The pharmaceutical composition of embodiment 126, wherein an abatacept/aldesleukin dose comprises about 29.17 mg abatacept and about 1×10⁶ units aldesleukin.
131. The pharmaceutical composition of embodiment 126, wherein an abatacept/aldesleukin dose comprises about 29.17 mg abatacept and about 1×10⁷ units aldesleukin.
132. The pharmaceutical composition of embodiment 126, wherein an abatacept/aldesleukin dose comprises about 5 mg to about 50 mg abatacept and about 3×10⁴ to about 3×10⁷ units aldesleukin.
133. The pharmaceutical composition of embodiment 126, wherein an abatacept/aldesleukin dose comprises about 16.67 mg abatacept and about 1×10⁵ units aldesleukin.
134. The pharmaceutical composition of embodiment 126, wherein an abatacept/aldesleukin dose comprises about 16.67 mg abatacept and about 1×10⁶ units aldesleukin.
135. The pharmaceutical composition of embodiment 126, wherein an abatacept/aldesleukin dose comprises about 16.67 mg abatacept and about 1×10⁷ units aldesleukin.
136. The pharmaceutical composition of embodiment 126, wherein an abatacept/aldesleukin dose comprises about 12.5 mg to about 125 mg abatacept and about 3×10⁴ to about 3×10⁷ units aldesleukin.
137. The pharmaceutical composition of embodiment 126, wherein an abatacept/aldesleukin dose comprises about 41.67 mg abatacept and about 1×10⁵ units aldesleukin.
138. The pharmaceutical composition of embodiment 126, wherein an abatacept/aldesleukin dose comprises about 41.67 mg abatacept and about 1×10⁶ units aldesleukin.
139. The pharmaceutical composition of embodiment 126, wherein an abatacept/aldesleukin dose comprises comprising about 41.67 mg abatacept and about 1×10⁷ units aldesleukin.
140. The pharmaceutical composition of embodiment 126, wherein the pharmaceutical compositions comprises one or more abatacept/aldesleukin doses as shown at Table 4.
141. The pharmaceutical composition of any one of embodiments 126-140, wherein the pharmaceutical composition is present in lyophilized form.
142. The pharmaceutical composition of embodiment 141, wherein the pharmaceutical composition is present as a lyophilized powder or lyophilized cake.
143. The pharmaceutical composition of any one of embodiments 126-140, wherein the pharmaceutical composition is a solution.
144. The pharmaceutical composition of embodiment 143, wherein the pharmaceutical composition is present as an aqueous solution.
145. The pharmaceutical composition of embodiment 143 or 144, wherein the one or more abatacept/aldesleukin doses are present at a concentration of 1 abatacept/aldesleukin dose/0.4 ml.
146. The pharmaceutical composition of embodiment 143 or 144, wherein the one or more abatacept/aldesleukin doses are present at a concentration of 1 abatacept/aldesleukin dose/0.7 ml.
147. The pharmaceutical composition of embodiment 143 or 144, wherein the one or more abatacept/aldesleukin doses are present at a concentration of 1 abatacept/aldesleukin dose/1.0 ml.
148. The pharmaceutical composition of embodiment 143 or 144, wherein the one or more abatacept/aldesleukin doses are present at a concentration of 1 abatacept/aldesleukin dose/1.5 ml.
149. The pharmaceutical composition of embodiment 143 or 144, wherein the one or more abatacept/aldesleukin doses are present at a concentration of 1 abatacept/aldesleukin dose/2.0 ml.
150. The pharmaceutical composition of any one of embodiments 126-149, wherein the pharmaceutical composition is suitable for subcutaneous administration.
151. The pharmaceutical composition of any one of embodiments 126-149, wherein the pharmaceutical composition is suitable for intravenous administration.

### 4. BRIEF DESCRIPTION OF THE FIGURES

FIG. 1: Dose-dependent suppression of M1 IL-6 expression in pro-inflammatory M1 macrophages with increasing amounts of a CTLA4 IgG (abatacept).

FIG. 2: Dose-dependent suppression of Tresp proliferation with increasing amounts of a CTLA4 IgG (abatacept).

FIG. 3: Dose-dependent enhanced suppressive function of Tregs on Tresp proliferation of IL-2 induced in vivo expanded Tregs isolated from Alzheimer patients with increasing amounts of a CTLA4 IgG (abatacept).

FIG. 4: Dose-dependent enhanced Treg suppression of M1 IL6 protein expression of IL-2 induced in vivo expanded Tregs (isolated from Alzheimer patients):M1 co-culture with increasing amounts of a CTLA4 IgG (abatacept).

FIG. 5: Impact on Treg suppression of M1-IL6 protein expression percentage of Tregs isolated from Alzheimer patients co-cultured with M1 upon the addition of IL-2 or a CTLA4 IgG (abatacept), or a combination thereof.

FIG. 6: Effect of IL-2 and abatacept treatment in restoring Treg function in Patient AD-01.

FIG. 7: Effect of IL-2 and abatacept treatment in restoring Treg function in Patient AD-02.

FIG. 8: Effect of IL-2 and abatacept treatment in cognitive improvement (MMSE score) in Patient AD-01 and Patient AD-02.

FIG. 9: Effect of IL-2 monotherapy in MMSE score in AD patients (n=8), and effect of IL-2 and abatacept treatment in MMSE score in AD patients (n=3). “Screening” refers to the measurement taken prior to initiation of the dosing regimens.

FIG. 10: Effect of IL-2 monotherapy on Treg suppressive function in AD patients (n=8; left graph), and effect of IL-2 and abatacept treatment on Treg suppressive function in AD patients (n=3; right graph). F/U=follow-up, post-treatment. Baseline and SC are measurements taken just prior to the initiation of the dosing regimens. For the IL-2 plus abatacept graph, the D8 measurement (showing an approximately 19% change over SC) was taken following the initial abatacept-only dose.

FIG. 11: Effect of IL-2 and abatacept treatment on Treg suppressive function in ALS patients.

FIG. 12: Effect of IL-2 and abatacept treatment on the percentage of cells expressing CD4+CD25+ FOXP3+ in ALS patients.

FIG. 13: Effect of IL-2 and abatacept treatment on the percentage of cells expressing CD8+ in ALS patients.

FIG. 14: ALSFRS-R scores in ALS patients prior to and during treatment with IL-2 and abatacept.

FIG. 15: Maximum inspiratory pressure (MIP) values in ALS patients prior to and during (shaded) treatment with IL-2 and abatacept.

### 5. DETAILED DESCRIPTION

In one aspect, provided herein are methods of treating a disease or disorder, for example a neurodegenerative or neuroinflammatory disease or disorder, e.g., Alzheimer's disease, comprising administering to a subject in need of treatment i) a CTLA-4-containing protein, and ii) an IL-2 protein, wherein the method mitigates one or more symptoms associated with the disease or disorder. In certain embodiments, the CTLA-4-containing protein is abatacept. In certain embodiments, the IL-2 protein is aldesleukin. In certain embodiments, the CTLA-4-containing protein is abatacept and the IL-2 protein is aldesleukin.

In certain embodiments, the CTLA-4-containing protein and the IL-2 protein are administered to the subject separately. In certain embodiments, the CTLA-4-containing protein is abatacept, and the abatacept and the IL-2 protein are administered to the subject separately. In certain embodiments, the IL-2 protein is aldesleukin, and the aldesleukin and the CTLA-4-containing protein are administered to the subject separately. In certain embodiments, the CTLA-4-containing protein is abatacept and the IL-2 protein is aldesleukin, and the abatacept and the aldesleukin are administered to the subject separately.

In certain embodiments, the CTLA-4-containing protein and the IL-2 protein are administered to the subject together in a single formulation. In certain embodiments, the CTLA-4-containing protein is abatacept, and the abatacept and the IL-2 protein are administered to the subject together in a single formulation. In certain embodiments, the IL-2 protein is aldesleukin, and the aldesleukin and the CTLA-4-containing protein are administered to the subject together in a single formulation. In certain embodiments, the CTLA-4-containing protein is abatacept and the IL-2 protein is aldesleukin, and the abatacept and the aldesleukin are administered to the subject together in a single formulation.

Also presented herein are pharmaceutical composition comprising one or more doses of a CTLA-4-containing protein and an IL-2 protein (“CTLA-4-containing protein/IL-2 protein doses”). In certain embodiments, presented herein is a pharmaceutical composition comprising one or more doses of abatacept and aldesleukin (“abatacept/aldesleukin doses”).

In another aspect, presented herein is a kit, comprising, in separate containers, i) one or more doses of a formulation comprising 50 to 125 mg abatacept, and ii) one or more doses of a formulation comprising 500,000 to 3,000,000 units aldesleukin. In certain embodiments, the kit comprises one or more doses of a formulation comprising 50 mg abatacept, 87.5 mg abatacept or 125 mg abatacept. In specific embodiments, the one or more doses of abatacept are present in lyophilized form, e.g., are present as a lyophilized powder or cake. In certain embodiments, the formulation of one or more doses of abatacept is suitable for subcutaneous administration or intravenous administration. In certain embodiments, the kit comprises one or more doses of a formulation comprising 500,000 to 2,000,000 units aldesleukin or 1,000,000 units aldesleukin. In specific embodiments, the one or more doses of aldesleukin are present in lyophilized form, e.g., are present as a lyophilized powder or cake. In certain embodiments, the formulation of one or more doses of aldesleukin is suitable for subcutaneous administration or intravenous administration.

Recitation of ranges of values herein are merely intended to serve as a shorthand method of referring individually to each separate value falling within the range, unless otherwise indicated herein, and each separate value is incorporated into the specification as if it were individually recited herein.

Unless specifically stated or apparent from context, as used herein, the terms “a”, “an”, and “the” are understood to be singular or plural, and denote “one or more.”

The terms “include,” “such as,” and the like are intended to convey inclusion without limitation, unless otherwise specifically indicated.

The terms “or” and “and” can be used interchangeably and can be understood to mean “and/or.”

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range. In particular, unless otherwise noted the terms mean within plus or minus 10% of a given value or range. In instances where an integer is required, the terms mean within plus or minus 10% of a given value or range, rounded either up or down to the nearest integer.

### 5.1 CTLA-4-Containing Proteins

The methods and compositions presented herein comprise or utilize a CTLA-4-containing protein, for example, a human CTLA-4-containing protein.

CTLA-4 (cytotoxic T-lymphocyte associated protein 4) proteins are well known. See, e.g., UniProtKB identifier P16410.

In certain embodiments, the CTLA-4-containing protein is a human CTLA-4-containing protein. In certain embodiments, the CTLA-4-containing protein comprises a CD80- and/or CD86-binding portion of CTLA-4. In certain embodiments, the CTLA-4-containing protein comprises a human CTLA-4 extracellular domain. In particular embodiments, the CTLA-4-containing protein comprises the extracellular domain of the following sequence:

(SEQ ID NO: 4)
KAMHVAQPAVVLASSRGIASFVCEYASPGKATEVRVTVLRQADSQVTEV
CAATYMMGNELTFLDDSICTGTSSGNQVNLTIQGLRAMDTGLYICKVEL
MYPPPYYLGIGNGTQIYVIDPEPCPDSDFLLWILAAVSSGLFFYSFLLT
AVSLSKMLKKRSPLTTGVYVKMPPTEPECEKQFQPYFIPIN.

For example, in certain embodiments, the CTLA-4-containing protein comprises the underlined portion of SEQ ID NO:4. In other embodiments, the CTLA-4-containing protein comprises at least 80%, 85%, 90%, 95%, 98%, 99% of the underlined portion of SEQ ID NO:4. In other embodiments, the CTLA-4-containing protein comprises at least 80%, 85%, 90%, 95%, 98%, 99% of the bolded and underlined portion of SEQ ID NO:4. In particular embodiments, for example, a CTLA-4-containing protein may comprise a sequence that is at least 90%, 95%, 98% or 99% identical to the amino acid sequence of SEQ ID NO:4, the underlined portion of SEQ ID NO:4 or the bolded and underlined portion of SEQ ID NO:4

To determine the percent identity of two amino acid sequences, the sequences are aligned for optimal comparison purposes (e.g., gaps can be introduced in the sequence of a first amino acid sequence for optimal alignment with a second amino acid sequence). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. In a certain embodiment, the percent identity is determined over the entire length of an amino acid sequence.

The determination of percent identity between two sequences (e.g., amino acid sequences) can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the XBLAST program of Altschul et al., 1990, J. Mol. Biol. 215:403. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST) can be used (see, e.g., National Center for Biotechnology Information (NCBI) on the worldwide web, ncbi.nlm.nih.gov). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, the CTLA-4-containing protein is a monomer. In certain embodiments, the CTLA-4-containing protein is a dimer.

In particular embodiments, the CTLA-4-containing protein is a fusion protein, for example, a fusion protein that comprises a human CTLA-4 extracellular domain, such as those described herein, and a human immunoglobulin Fe domain, e.g., a modified Fe domain that comprises an immunoglobulin hinge region, CH2 region and CH3. In particular embodiments, the human immunoglobulin Fe domain is an Ig domain, for example, a human IgG1 Fe domain. In certain embodiments, the CTLA-4-containing protein is glycosylated.

In certain embodiments, the CTLA-4-containing protein comprises the following amino acid sequence monomer:

(SEQ ID NO: 1)
MHVAQPAVVLASSRGIASFVCEYASPGKATEVRVTVLRQADSQVTEVCA
ATYMMGNELTFLDDSICTGTSSGNQVNLTIQGLRAMDTGLYICKVELMY
PPPYYLGIGNGTQIYVIDPEPCPDSDQEPKSSDKTHTSPPSPAPELLGG
SSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGK.

In specific embodiments, the CTLA-4-containing protein comprises a homodimer of two monomers, each monomer comprising the amino acid sequence of SEQ ID NO:1.

In particular embodiments, the CTLA-4-containing protein is abatacept.

In certain embodiments, the CTLA-4-containing protein comprises the following amino acid sequence monomer:

(SEQ ID NO: 2)
MHVAQPAVVLASSRGIASFVCEYASPGKYTEVRVTVLRQADSQVTEVCA
ATYMMGNELTFLDDSICTGTSSGNQVNLTIQGLRAMDTGLYICKVELMY
PPPYYEGIGNGTQIYVIDPEPCPDSDQEPKSSDKTHTSPPSPAPELLGG
SSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGK.

In specific embodiments, the CTLA-4-containing protein comprises a homodimer of two monomers, each monomer comprising the amino acid sequence of SEQ ID NO:2.

In particular embodiments, the CTLA-4-containing protein is belatacept.

In certain embodiments, the CTLA-4-containing protein is glycosylated.

### 5.2 IL-2 Proteins

The methods and compositions presented herein comprise or utilize an IL-2 protein, for example, a human IL-2 protein.

IL-2 proteins are well known. See, e.g., UniProtKB identifier QOGK43.

In certain embodiments, the IL-2 protein is a human IL-2 protein. For example, in certain embodiments, the IL-2 protein is or is derived from the following native mature human IL-2 amino acid sequence:

(SEQ ID NO: 5)
APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFCQSIISTLT.

In certain embodiments, the IL-2 protein contains one or more mutations relative to the native mature human IL-2 polypeptide. For example, in certain embodiments, the human IL-2 protein lacks an N-terminal alanine amino acid. In particular embodiments, the human IL-2 protein comprises a mutation at the amino acid position corresponding to native mature human Il-2 amino acid residue 125 (underlined in the above sequence). For example, in particular embodiments, the IL-2 protein contains a serine at the amino acid position corresponding to native mature human IL-2 amino acid residue 125. In particular embodiments, the human IL-2 protein lacks an N-terminal alanine amino acid and comprises a serine at the amino acid position corresponding to native mature human IL-2 amino acid residue 125.

In specific embodiments, the IL-2 protein is not glycosylated.

In particular embodiments, the human IL-2 protein lacks an N-terminal alanine amino acid, comprises a serine at the amino acid position corresponding to native mature human IL-2 amino acid residue 125 and is not glycosylated.

In certain embodiments, the IL-2 protein is aldesleukin (des-alanyl-1, serine-125 human interleukin-2; trade name PROLEUKIN), which is well-known.

In certain embodiments, the human IL-2 protein comprises the following amino acid sequence:

(SEQ ID NO: 6)
PTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGS
ETTFMCEYADETATIVEFLNRWITFSQSIISTLT.

In certain embodiments, the IL-2 protein comprises one or modifications, e.g., mutations, compared to that of mature human IL-2 and exhibits reduced binding to IL-2 receptor (IL-2R) alpha subunit (CD25) relative to that of native mature IL-2 protein. In certain embodiments, the IL-2 protein comprises one or modifications, e.g., mutations, compared to that of mature human IL-2 and exhibits reduced binding to Il-2 receptor beta subunit (CD122) relative to that of native IL-2 protein. In certain embodiments, the IL-2 protein comprises one or modifications, e.g., mutations, compared to that of mature human IL-2 and reduced binding to IL-2 receptor gamma subunit (CD132) relative to that of wild-type IL-2 protein. In particular embodiments, the IL-2 protein comprises one or modifications, e.g., mutations, compared to that of mature human IL-2 and exhibits reduced binding to IL-2 receptor beta and gamma subunits relative to that of native mature IL-2 protein. In particular embodiments, the IL-2 protein comprises one or modifications, e.g., mutations, compared to that of mature human IL-2 and exhibits reduced binding to IL-2 receptor alpha and beta subunits relative to that of native mature IL-2 protein. In particular embodiments, the IL-2 protein comprises one or modifications, e.g., mutations, compared to that of mature human IL-2 and exhibits reduced binding to IL-2 receptor beta subunit but not reduced binding to IL-2 receptor subunit alpha relative to that of native mature IL-2 protein. In particular embodiments, the IL-2 protein comprises one or modifications, e.g., mutations, compared to that of mature human IL-2 and exhibits reduced binding to IL-2 receptor alpha, beta and gamma subunits relative to that of native mature IL-2 protein.

In certain embodiments, the IL-2 protein comprises one or modifications, e.g., mutations, compared to that of mature human IL-2 and exhibits reduced IL-2 receptor-mediated signaling activity relative to that of native mature IL-2 protein. IL-2 receptor-mediated signaling activity may be assayed using routine, well known techniques, for example may be assayed via an assessment of STAT5 phosphorylation. See, e.g., Ghelani et al. (2020) Front. Immunol. 11:1106, which is incorporation herein in its entirety.

In certain embodiments, the IL-2 protein comprises one or modifications, e.g., mutations, compared to that of mature human IL-2 and exhibits selectivity for (e.g., preferential activation of) T regulatory (Treg) cells, for example selectivity over natural killer cells and/or T effector cells, as assessed, e.g., by Treg cell proliferation assays, Treg-mediated suppressor function, and/or lineage and/or phenotypic marker expression. Such assays are well-known. See, e.g., Ghelani (2020) Id.

In some embodiments, the IL-protein is an IL-2 mutein (that is, an IL-2 protein comprising one or more mutations relative to native mature IL-2 protein) comprising an amino acid sequence corresponding to native mature human IL-2, which further comprises a replacement substitution or deletion at one or more amino acid positions, e.g., A1 (deletion); P2 (e.g., deletion); T3 (e.g., T3C, T3A, T3G, T3Q, T3E, T3N, T3D, T3R, T3K, T3P, or deletion); S4 (e.g., deletion); S5 (e.g., deletion); S6 (e.g., deletion); H16 (e.g., H16E, H16R), L18 (e.g., L18R, L18G, L18M, L18F, L18E, L18H, L18W, L18K, L18Q, L18S, L18V, L18I, L18Y, L18H, L18D, L18T); D20 (e.g., D20A, D20G, D20H, D20W); Q22 (e.g., Q22F, Q22E, Q22G, Q22A, Q22L, Q22M, Q22F, Q22W, Q22K, Q22S, Q22V, Q22I, Q22Y, Q22H, Q22R, Q22N, Q22D, Q22T, Q22F); K35 (e.g., K35E); R38 (e.g., R38A, R38G); M39 (e.g., M39L, M39V); F42 (e.g., F42A, F42L, F42Y); Y45 e.g., Y45A); H55 (e.g., H55Y); C58 (e.g., deletion); E61 (e.g., E61Q); E62 (e.g., E62A); 186 (e.g., I86V); N88 (e.g., N88I, N88G, N88D, N88K, N88R); 189 (e.g., I89V); V91 (e.g., V91D, V91K); 192 (I92F); K97 (e.g, K97Q); M104 (e.g., M104A, M104T, M104V); D109 (e.g, D109C or substituted with a non-natural amino acid with an activated side chain); T113 (e.g, T113N); C125 (e.g., C125A, C125S); Q126 (e.g., Q126H, Q126M, Q126K, Q126C, Q126D, Q126E, Q126G, Q126I, Q126R, Q126S, Q126T); or S130 (e.g., S130T, S130G, S130R).

In certain embodiments, the human IL-2 protein comprises the following amino acid sequence:

(SEQ ID NO: 3)
PTSSSTKKTQLQLEHLLLDLQMILNGINNYKNPKLTRMLTFKFYMPKKA
TELKHLQLEEELKPLEEVLNLAQSKNFHLRPRDLISNINVIVLELKGSE
TTFMCEYADETATIVEFLNRWITFSQSIISTLT.

In some embodiments, the IL-protein is an IL-2 mutein that comprises an amino acid sequence corresponding to native mature human IL-2 and which further comprises D20A and H16E mutations; D20A and M104T mutations; H16E and E61Q mutations; V91K, D20A and M104V mutations; a D20G mutation; a D20W mutation; an F42Y mutation; an N88K mutation; or D20A, H16R and E61Q mutations.

In some embodiments, the IL-protein is an IL-2 mutein that comprises an amino acid sequence corresponding to native mature human IL-2 and which further comprises a D20, N88 and/or Q126 mutation. For example, in certain embodiments, an IL-2 protein may comprise a D20H mutation, an N88R, N88I or N88G mutation, and/or a Q126D mutation. See, e.g., U.S. Pat. No. 6,955,807.

In some embodiments, the IL-2 protein is an IL-2Ra/IL-2Rb biased IL-2 protein. For example, in certain embodiments, the IL-12 is STK-012 (Emmerich, J. et al. Cancer Res 2021; 81(13_Suppl):Abstract nr 1744.

In certain embodiments, the IL-2 protein is fused or conjugated to one or more additional moieties. In some embodiments, the IL-protein is fused or conjugated to one or more polymers, e.g., one or more polymers having a weight average molecular weight of from about 250 Daltons to about 50,000 Daltons. In some embodiments, the IL-2 protein is pegylated. See, e.g., WO202114636. In particular embodiments, the IL-2 protein or IL-2 mutein is pegylated at a tyrosine residue. In specific embodiments, the pegylated tyrosine residue is Y45 or F42Y. In some embodiments, the IL-2 protein is a modified IL-2 polypeptide as described in PCT Publication No. WO2021140416. In some embodiments, the IL-2 protein is IL-2 clinical candidate BPT-143 (Bright Peak).

In some embodiments, the IL-2 protein is conjugated to one or more water-soluble polymers. In some embodiments, the water-soluble polymer is conjugated at the unnatural amino acid. In some embodiments, the water-soluble polymer comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), poly[oligo(ethylene glycol)methyl methacrylate] (POEGMA), or a combination thereof. In specific embodiments, the water-soluble polymer is PEG and has a weight average molecular weight of from about 100 Daltons to about 150,000 Daltons.

In some embodiments, the IL-2 protein is conjugated via releasable linkage to one or more poly(ethylene)glycol (PEG) polymers, for example to one to seven PEG polymers, e.g., branched PEG. In particular embodiments, the PEG polymers are branched polymers each having a weight average molecular weight of from about 20,000 daltons to 85,000 daltons. In some embodiments, the releasable branched PEG is attached at an amino group of a lysine of the IL-2 protein. In some embodiments, there is a plurality of conjugates that is a mixture monopegylated, dipeglated and tripegylated conjugates. In some embodiments, the IL-2 protein is a conjugate of IL-2 protein as described in U.S. Pat. Nos. 9,861,705, 10,960,079 or PCT Publication No. WO2012065086. In some embodiments, the IL-2 protein is clinical candidate NKTR-214.

In some embodiments, the IL-2 protein is fused or conjugated to an antibody or fragment thereof. In some embodiments, the antibody or fragment thereof binds to human IL-2 (e.g., an anti-hIL-2 antibody). In some embodiments, the antibody binds to human IL-2Ra. In some embodiments, the antibody binds to human IL-2Ra. In some embodiments, the antibody is a whole antibody. In some embodiments, the antibody fragment is an antigen binding domain. In some embodiments, the antibody fragment is an Fc domain, for example, a human Fc domain, e.g., a human IgG Fc domain. In particular embodiments, the IL-2 protein comprises an N-terminal or C-terminal human Fc domain fusion or conjugation, e.g., a human IgG Fc domain. In some embodiments, such moieties are directly attached to the IL-2 protein. In other embodiments, such moieties are attached to the IL-2 protein indirectly, for example via linker, e.g., via a GSSSS-containing linker, for example, a GSSS, (GSSSS)₂, (GSSSS)₃ or (GSSSS)₄-containing liker.

In particular embodiments, the IL-protein is an IL-2 mutein that comprises an amino acid sequence corresponding to native mature human IL-2 and which further comprises: i) mutations at one or more of L53 (e.g., L53I), L56 (e.g., L56I), L80 (e.g., L80I), and L118 (e.g., L118I); and, optionally, mutations at one or more of V69 (e.g., V69A), Q74 (e.g., Q74P), N88 (e.g., N88D), and C125 (e.g., C125S), for example, L53I, N88D, V69A, Q74P, and C125S mutations; L56I, N88D, V69A, Q74P, and C125S mutations; L80I, N88D, V69A, Q74P, and C125S mutations; or L118I, N88D, V69A, Q74P, and C125S mutations; and ii) optionally an Fc domain, for example, a human IgG1 Fc domain, e.g., an N-terminal human IgG1 Fc domain.

In some embodiments, the IL-2 protein is an IL-2 mutein comprising: i) an amino acid sequence corresponding to native mature human IL-2, which further comprises a replacement substitution at one or more positions: E15 (e.g., E15Q); H16 (e.g., H16N); Q22 (e.g., Q22E); N29 (e.g., N29S); Y31 (e.g., Y31S, Y31H); K35 (e.g., K35R); T37 (e.g., T37A); K48 (e.g., K48E); V69 (e.g., V69A); N71 (e.g., N71R); Q74 (e.g., Q74P); D84 (e.g., D84N); N88 (e.g., N88D, N88R); E95 (e.g., E95Q); C125 (e.g., C125A, C125S); or Q126 (e.g., Q126E); and ii) optionally is fused or conjugated to an antibody or antigen-binding fragment that binds to MAdCAM, OAT1, OCT2, FXYD2, TSPAN7, DPP6, HEPACAM2, TMEM27, or GPR119.

In some embodiments, the IL-2 protein or IL-2 mutein, e.g., IL-2 mutein fused or conjugated one or more additional moieties, e.g., an antibody, antigen-binding fragment of an antibody, or an Fc domain, are as described in U.S. Pat. Nos. 10,174,091, 10,174,092, 10,946,068, 11,091,526, or 11,091,527 or PCT Publication Nos. WO2019112852 or WO2019112854.

In some embodiments, the IL-2 protein is PT101/MK-6194 (Pandion Therapeutics/Merck & Co).

In some embodiments, the IL-2 protein is an IL-2 mutein conjugated to an antibody or fragment thereof as described in PCT Publication No. WO2020247843.

In some embodiments, the IL-2 protein is IL-2 clinical candidate AB248.

In some embodiments, the IL-2 protein is ANV419 (Anaveon).

In certain embodiments, the IL-2 protein comprises one or more non-standard or non-natural amino acids. For example, in particular embodiments, the IL-2 protein may comprise an amino acid sequence corresponding to the mature human IL-2 protein and may further comprise one or more amino acids other than the standard twenty amino acids found in the majority of proteins.

For example, in some embodiments, the IL-protein is an IL-2 mutein comprising a homoserine (Hse) substitution at any one of residues 35-45, 61-81, or 94-114. In some embodiments, the IL-2 mutein comprises Hse41, Hse71, Hse104, or a combination thereof. In some embodiments, the IL-2 mutein comprises norleucine substitution at positions 23, 39, or 46.

In some embodiments, the IL-2 protein comprises at least one non-natural amino acid. In some embodiments, the at least one non-natural amino acid is a replacement substitution at an amino acid position corresponding to native mature human IL-2 selected from T37, R38, T41, F42, K43, F44, Y45, E60, E61, E62, K64, P65, E68, V69, N71, L72, M104, C105, or Y107. In some embodiments, the unnatural amino acid is a lysine analogue or comprises an aromatic side chain. In some embodiments, the unnatural amino acid is N6-[(2-azidoethoxy)carbonyl]-1-lysine.

In some embodiments, the IL-2 protein comprises at least one unnatural amino acid. In some embodiments, the at least one unnatural amino acid is a replacement substitution at an amino acid position corresponding to native mature human IL-2 selected from T37, R38, T41, F42, K43, F44, Y45, E60, E61, E62, K64, P65, E68, V69, N71, L72, M104, C105, or Y107. In some embodiments, the unnatural amino acid is a lysine analogue or comprises an aromatic side chain. In some embodiments, the unnatural amino acid is N6-[(2-azidoethoxy)carbonyl]-1-lysine. In some embodiments, the IL-2 protein is a conjugate of an IL-2 mutein comprising at least one unnatural amino acid as described in U.S. Pat. No. 10,610,571 or PCT Publication Nos. WO2019028419 or WO2019028425; PCT Publication No. WO19165453; U.S. Pat. No. 11,077,195 or PCT Publication No. WO2020163532; PCT Publication No. WO2021030706; PCT Publication No. WO2021050554; or PCT Publication No. WO2021263026. In some embodiments, the IL-2 protein is THOR-707 (Sanofi).

In some embodiments, the IL-2 protein is an IL-2 mimetic (e.g., a de novo protein that mimics the activity of IL-2). In some embodiments, the IL-2 protein is an IL-2 mimetic as described in PCT Publication No WO2021081193 or PCT Publication No WO2021188374. In some embodiments, the IL-2 mimetic induces the hetero-dimerization of two IL-2 cell membrane receptors. In some embodiments, the IL-2 mimetic is Neoleukin-2/15. In some embodiments, the IL-2 protein is IL-2 clinical candidate NL-201.

### 5.3 Methods of Treatment

Provided herein are methods of treating a disease or disorder, comprising administering to a subject in need of treatment i) a CTLA-4-containing protein, and ii) an IL-2 protein, wherein the method mitigates one or more symptoms associated with the disease or disorder. In certain embodiments, the CTLA-4-containing protein is abatacept. In certain embodiments, the IL-2 protein is aldesleukin. In certain embodiments, the CTLA-4-containing protein is abatacept and the IL-2 protein is aldesleukin.

In certain embodiments, the CTLA-4-containing protein and the IL-2 protein are administered to the subject separately. In certain embodiments, the CTLA-4-containing protein is abatacept, and the abatacept and the IL-2 protein are administered to the subject separately. In certain embodiments, the IL-2 protein is aldesleukin, and the aldesleukin and the CTLA-4-containing protein are administered to the subject separately. In certain embodiments, the CTLA-4-containing protein is abatacept and the IL-2 protein is aldesleukin, and the abatacept and the aldesleukin are administered to the subject separately.

In certain embodiments, the CTLA-4-containing protein and the IL-2 protein are administered to the subject together in a single formulation. In certain embodiments, the CTLA-4-containing protein is abatacept, and the abatacept and the IL-2 protein are administered to the subject together in a single formulation. In certain embodiments, the IL-2 protein is aldesleukin, and the aldesleukin and the CTLA-4-containing protein are administered to the subject together in a single formulation. In certain embodiments, the CTLA-4-containing protein is abatacept and the IL-2 protein is aldesleukin, and the abatacept and the aldesleukin are administered to the subject together in a single formulation.

In some embodiments, the disease or disorder is associated with Treg dysfunction and the subject is diagnosed with or is suspected of having a disorder associated with Treg dysfunction. In some embodiments, the disease or disorder is associated with Treg deficiency and the subject is diagnosed with or is suspected of having a disorder associated with Treg deficiency. In some embodiments, the disease or disorder is a condition driven by a T cell response and the subject is diagnosed with or is suspected of having a condition driven by a T cell response.

In some embodiments the disease is a neurodegenerative disease and the subject is diagnosed with or is suspected of having a neurodegenerative disease. In some embodiments, the subject is diagnosed with or is suspected of having Alzheimer's disease, Amyotrophic Lateral Sclerosis, Huntington's disease, Parkinson's disease, or frontotemporal dementia.

In some embodiments, the disorder is a disorder that would benefit from downregulation of the immune system, and the subject is diagnosed with or is suspected of having a disorder that would benefit from downregulation of the immune system.

In some embodiments, the disease is an autoimmune disease, and the subject is diagnosed with or suspected of having an autoimmune disease. The autoimmune disease may be, for example, systemic sclerosis (scleroderma), polymyositis, ulcerative colitis, inflammatory bowel disease, Crohn's disease, celiac disease, multiple sclerosis (MS), rheumatoid arthritis (RA), Type I diabetes, psoriasis, dermatomyositis, systemic lupus erythematosus, cutaneous lupus, myasthenia gravis, autoimmune nephropathy, autoimmune hemolytic anemia, autoimmune cytopenia autoimmune hepatitis, autoimmune uveitis, alopecia, thyroiditis or pemphigus.

In certain embodiments, the disease is moderately to severely active RA. In certain embodiments, the disease is moderately to severely active RA and the subject is an adult. In certain embodiments, the disease is polyarticular juvenile idiopathic arthritis (pJIA). In particular, embodiments, the disease is pJIA and the subject is 2 years of age or older, for example, 6 years of age or older. In certain embodiments, the disease is psoriatic arthritis. In particular embodiments, the disease is psoriatic arthritis and the subject is an adult.

In some embodiments, the disease or disorder is heart failure or ischemic cardiomyopathy, and the subject is diagnosed with or suspected of having heart failure or ischemic cardiomyopathy. In some embodiments, the disease is graft-versus-host disease, and the subject is diagnosed with or suspected of having graft-versus-host disease, e.g., after undergoing organ transplantation (such as a kidney transplantation or a liver transplantation), or after undergoing stem cell transplantation (such as hematopoietic stem cell transplantation).

In some embodiments, the disease or disorder is neuroinflammation or is a disease or disorder associated with neuroinflammation, and the subject is diagnosed with or suspected of having neuroinflammation. The neuroinflammation may be associated, for example, with stroke, acute disseminated encephalomyelitis (ADEM), acute optic neuritis, transverse myelitis, neuromyelitis optica (NMO), epilepsy, traumatic brain injury, spinal cord injury, encephalitis central nervous system (CNS) vasculitis, neurosarcoidosis, autoimmune or post-infectious encephalitis, or chronic meningitis.

In some embodiments, the disease or disorder is cardio-inflammation, and the subject is diagnosed with or suspected of having cardio-inflammation, e.g., cardio-inflammation associated with atherosclerosis, myocardial infarction, ischemic cardiomyopathy, with heart failure.

In some embodiments, disease or disorder is chronic inflammatory demyelinating polyradiculoneuropathy (CIDP), and the subject is diagnosed with or suspected of having CIDP. In some embodiments, the disease or disorder is acute inflammatory demyelinating polyneuropathy (AIDP), and the subject is diagnosed with or suspected of having AIDP. In some embodiments, the disease or disorder is Guillain-Barre syndrome (GBS), and the subject is diagnosed with or suspected of having GBS.

In some embodiments, the subject has had a stroke.

In some embodiments, the subject being treated is diagnosed with or suspected of having cancer, e.g., a blood cancer.

In some embodiments, the subject being treated is diagnosed with or suspected of having asthma.

In some embodiments, the subject being treated is diagnosed with or suspected of having eczema.

In some embodiments, the subject being treated is diagnosed with or suspected of having a disorder associated with overactivation of the immune system.

In some embodiments, the subject being treated is diagnosed with or suspected of having Tregopathy. The Tregopathy may be caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In one aspect, presented herein is a method of treating a disease or disorder in a subject in need thereof, for example, a disease or disorder described herein, e.g., a neurodegenerative or neuroinflammatory disease or disorder, comprising administering to the subject:
i) a CTLA-4-containing protein; and
ii) an IL-2 protein;
wherein the method mitigates one or more symptoms associated with the neurodegenerative or neuroinflammatory disease or disorder in the treated subject. In certain embodiments, the CTLA-4-containing protein is abatacept. In certain embodiments, the IL-2 protein is aldesleukin. In certain embodiments, the CTLA-4-containing protein is abatacept and the IL-2 protein is aldesleukin.

In certain embodiments, the CTLA-4-containing protein is administered by injection or infusion. In particular embodiments, the CTLA-4-containing protein is administered subcutaneously. In particular embodiments, the CTLA-4-containing protein is administered intravenously. In certain embodiments, the IL-2 protein is administered by injection or infusion. In particular embodiments, the IL-2 protein is administered subcutaneously. In particular embodiments, the IL-2 protein is administered intravenously. In certain embodiments, the CTLA-4-containing protein and the IL-2 protein are administered by injection or infusion. In particular embodiments, the CTLA-4-containing protein and the IL-2 protein are administered subcutaneously. In particular embodiments, the CTLA-4-containing protein and the IL-2 protein are administered intravenously.

In certain embodiments, the CTLA-4-containing protein comprises a human CTLA-4 extracellular domain. In particular embodiments, the CTLA-4-containing protein is a fusion protein, for example, a fusion protein that comprises a human CTLA-4 extracellular domain and a human immunoglobulin Fc domain, e.g., a modified Fc domain that comprises an immunoglobulin hinge region, CH2 region and CH3. In particular embodiments, the human immunoglobulin Fc domain is a human IgG1 Fc domain. In certain embodiments, the CTLA-4-containing protein is glycosylated.

In certain embodiments, the CTLA-4-containing protein the following amino acid sequence monomer:

(SEQ ID NO: 1)
MHVAQPAVVLASSRGIASFVCEYASPGKATEVRVTVLRQADSQVTEVCA
ATYMMGNELTFLDDSICTGTSSGNQVNLTIQGLRAMDTGLYICKVELMY
PPPYYLGIGNGTQIYVIDPEPCPDSDQEPKSSDKTHTSPPSPAPELLGG
SSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGK.

In specific embodiments, the CTLA-4-containing protein comprises a homodimer of two monomers, each monomer comprising the amino acid sequence of SEQ ID NO:1.

In particular embodiments, the CTLA-4-containing protein is abatacept.

In certain embodiments, the IL-2 protein is a human IL-2 protein. In particular embodiments, the human IL-2 protein comprises a serine at the amino acid position corresponding to native mature human IL-2 amino acid residue 125. In certain embodiments, the human IL-2 protein lacks an N-terminal alanine amino acid. In particular embodiments, the human IL-2 protein lacks an N-terminal alanine amino acid and comprises a serine at the amino acid position corresponding to native mature human IL-2 amino acid residue 125.

In certain embodiments, the human IL-2 protein comprises the following amino acid sequence:

(SEQ ID NO: 3)
PTSSSTKKTQLQLEHLLLDLQMILNGINNYKNPKLTRMLTFKFYMPKKA
TELKHLQLEEELKPLEEVLNLAQSKNFHLRPRDLISNINVIVLELKGSE
TTFMCEYADETATIVEFLNRWITFSQSIISTLT.

In specific embodiments, the IL-2 protein is not glycosylated. In certain embodiments, the IL-2 protein is aldesleukin.

In certain embodiments, the human IL-2 protein comprises the following amino acid sequence:

(SEQ ID NO: 6)
PTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGS
ETTFMCEYADETATIVEFLNRWITFSQSIISTLT.

In some embodiments, according to the methods provided herein, the CTLA-4-containing protein is administered to the subject once every two weeks. In some embodiments, the CTLA-4-containing protein is administered intravenously to the subject once every two weeks. In some embodiments, the CTLA-4-containing protein is administered subcutaneously to the subject once every two weeks. In some embodiments, the CTLA-4-containing protein is administered to the subject once every two weeks for 10-20 weeks. For example, in some embodiments, the CTLA-4-containing protein is administered to the subject once every two weeks for 12 weeks, for 15 weeks, or for 18 weeks. In some embodiments, the CTLA-4-containing protein is abatacept.

In some embodiments, according to the methods provided herein, the CTLA-4-containing protein is administered to the subject once every week. In some embodiments, the CTLA-4-containing protein is administered intravenously to the subject once every week. In some embodiments, the CTLA-4-containing protein is administered subcutaneously to the subject once every week. In some embodiments, the CTLA-4-containing protein is administered to the subject once every weeks for 10-20 weeks. For example, in some embodiments, the CTLA-4-containing protein is administered to the subject once week for 12 weeks, for 15 weeks, or for 18 weeks. In some embodiments, the CTLA-4-containing protein is abatacept.

In some embodiments, according to the methods provided herein, the CTLA-4-containing protein is administered to the subject once daily for 2-5 consecutive days. For example, in some embodiments, the CTLA-4-containing protein is administered to the subject once daily for 2, 3, 4 or 5 consecutive days. In some embodiments, the CTLA-4-containing protein is administered to the subject once daily for three consecutive days. In some embodiments, the CTLA-4-containing protein is administered intravenously to the subject once daily for 2-5 consecutive days. In some embodiments, the CTLA-4-containing protein is administered subcutaneously to the subject once daily for 2-5 consecutive days. For example, in some embodiments, the CTLA-4-containing protein is administered subcutaneously to the subject once daily for 2, 3, 4 or 5 consecutive days. In some embodiments, the CTLA-4-containing protein is administered subcutaneously to the subject once daily for three consecutive days. In some embodiments, the IL-2 protein is administered to the subject once daily for 2-5 consecutive days. For example, in some embodiments, the IL-2 protein is administered to the subject once daily for 2, 3, 4 or 5 consecutive days. In some embodiments, the IL-2 protein is administered to the subject once daily for three consecutive days. In some embodiments, the IL-2 protein is administered intravenously to the subject once daily for 2-5 consecutive days. In some embodiments, the IL-2 protein is administered subcutaneously to the subject once daily for 2-5 consecutive days. For example, in some embodiments, the IL-2 protein is administered subcutaneously to the subject once daily for 2, 3, 4 or 5 consecutive days. In some embodiments, the IL-2 protein is administered subcutaneously to the subject once daily for three consecutive days. In some embodiments, the CTLA-4-containing protein is abatacept. In some embodiments, the IL-2 protein is aldesleukin.

In some embodiments, according to the methods provided herein, the CTLA-4-containing protein is administered to the subject one or more times during a dosing cycle, for example 1-10 times during a dosing cycle, such as 1, 2, 3, 4, 5, 6, 7, 8, 9 or 10 times during a dosing cycle. In some embodiments, the CTLA-4-containing protein is administered to the subject as a single administration on day 1 of the dosing cycle. In some embodiments, the CTLA-4-containing protein is administered to the subject daily for a series of consecutive days, beginning on day 1 of the dosing cycle. In some embodiments, the CTLA-4-containing protein is administered to the subject daily for two consecutive days, beginning on day 1 of the dosing cycle. In some embodiments, the CTLA-4-containing protein is administered to the subject daily for three consecutive days, beginning on day 1 of the dosing cycle. In some embodiments, the CTLA-4-containing protein is administered to the subject daily for four consecutive days, beginning on day 1 of the dosing cycle. In some embodiments, the CTLA-4-containing protein is administered to the subject daily for five consecutive days, beginning on day 1 of the dosing cycle. In some embodiments, the CTLA-4-containing protein is administered to the subject for a series of non-consecutive days, beginning on day 1 of the dosing cycle, for example, administered to the subject for a series of non-consecutive days, such as 2, 3, 4, 5, 6, 7, 8, 9 or 10 non-consecutive days, independently separated by 1, 2, 3, 4 or 5 days, beginning on day 1 of the dosing cycle. In some embodiments, the CTLA-4-containing protein is abatacept.

In some embodiments, according to the methods provided herein, the IL-2 protein is administered to the subject one or more times during a dosing cycle, for example 1-10 times during a dosing cycle, such as 1, 2, 3, 4, 5, 6, 7, 8, 9 or 10 times during a dosing cycle. In some embodiments, the IL-2 protein is administered to the subject daily for a series of consecutive days, beginning on day 1 of the dosing cycle. In some embodiments, the IL-2 protein is administered to the subject daily for two consecutive days, beginning on day 1 of the dosing cycle. In some embodiments, the IL-2 protein is administered to the subject daily for three consecutive days, beginning on day 1 of the dosing cycle. In some embodiments, the IL-2 protein is administered to the subject daily for four consecutive days, beginning on day 1 of the dosing cycle. In some embodiments, the IL-2 protein is administered to the subject daily for five consecutive days, beginning on day 1 of the dosing cycle. In some embodiments, the IL-2 protein is administered to the subject for a series of non-consecutive days, beginning on day 1 of the dosing cycle, for example, administered to the subject for a series of non-consecutive days, such as 2, 3, 4, 5, 6, 7, 8, 9 or 10 non-consecutive days, independently separated by 1, 2, 3, 4 or 5 days, beginning on day 1 of the dosing cycle. In some embodiments, the IL-2 protein is aldesleukin.

In some embodiments, according to the methods provided herein, the dosing cycle is 1-6 weeks. In some embodiments, according to the methods provided herein, the dosing cycle is 2-6 weeks. In some embodiments, the dosing cycle is 1 week. In some embodiments, the dosing cycle is 2 weeks. In some embodiments, the dosing cycle is 3 weeks. In some embodiments, the dosing cycle is 4 weeks. In some embodiments, the dosing cycle is 5 weeks. In some embodiments, the dosing cycle is 6 weeks. In some embodiments, the dosing cycle is repeated 1-12 times. In some embodiments, the dosing cycle is repeated 10 times. In some embodiments, the dosing cycle is repeated 8 times. In some embodiments, the dosing cycle is repeated 6 times. In some embodiments, each repeated dosing cycle begins 10-28 days after day 1 of the previous dosing cycle. For example, in some embodiments, each repeated dosing cycle begins 14 days after day 1 of the previous dosing cycle. In some embodiments, each repeated dosing cycle begins 2-6 weeks after day 1 of the previous dosing cycle. In some embodiments, each repeated dosing cycle begins 2 weeks after day 1 of the previous dosing cycle. In some embodiments, each repeated dosing cycle begins 3 weeks after day 1 of the previous dosing cycle. In some embodiments, each repeated dosing cycle begins 4 weeks after day 1 of the previous dosing cycle. In some embodiments, each repeated dosing cycle begins 5 weeks after day 1 of the previous dosing cycle. In some embodiments, each repeated dosing cycle begins 6 weeks after day 1 of the previous dosing cycle. In some embodiments, the CTLA-4-containing protein is administered to the subject daily for three consecutive days, beginning on day 1 of a first dosing cycle. In some embodiments, the IL-2 protein is administered to the subject daily for three consecutive days, beginning on day 1 of a first dosing cycle. In some embodiments, the IL-2 protein is administered to the subject daily for three consecutive days, beginning on day 1 of the second dosing cycle. In some embodiments, the first dosing cycle is repeated 6 times, with each repeated dosing cycle beginning 14 days after day 1 of the previous dosing cycle. In some embodiments, the CTLA-4-containing protein is abatacept. In some embodiments, the IL-2 protein is aldesleukin.

In some embodiments, the CTLA4-containing protein is administered to the subject weekly. In certain embodiments, the CTLA4-containing protein is administered to the subject weekly on day 1 of each week (in other words, if the first administration of the CTLA4-containing protein is administered to the subject on day 1, subsequent administrations of the CTLA4-containing protein occur at day 8, day 15, day 22 etc.). In some embodiments, the CTLA-4-containing protein is abatacept.

In specific embodiments, the CTLA4-containing protein is administered to the subject weekly and the IL-2 protein is administered to the subject every two weeks. For example, in certain embodiments, the CTLA4-containing protein is administered to the subject weekly beginning with week 1, and the IL-2 protein is administered to the subject on week 2, week 4, week 6 etc. For example, in certain embodiments, the CTLA4-containing protein is administered to the subject weekly beginning with week 1, and the IL-2 protein is administered to the subject on week 3, week 5, week 7 etc. In some embodiments, the CTLA-4-containing protein is abatacept. In some embodiments, the IL-2 protein is aldesleukin.

In certain embodiments, the CTLA4-containing protein is administered to the subject weekly on day 1 of each week (in other words, if the first administration of the CTLA4-containing protein is administered to the subject on day 1, subsequent administrations of the CTLA4-containing protein occur at day 8, day 15, day 22 etc.) and the IL-2 protein is administered to the subject on, or beginning on, day 1 of every other week, beginning with week 2 (in other words, day 8, day 22, day 36 etc). In certain embodiments, IL-2 protein is administered to the subject on day 1 of an IL-2 protein administration week for a series of consecutive days, e.g., daily for two consecutive days, daily for three consecutive days, daily for four consecutive days or daily for five consecutive days. In certain embodiments, IL-2 protein is administered to the subject on day 1 of an IL-2 protein administration week for a series of non-consecutive days, such as 2, 3, 4, 5, 6 or 7 non-consecutive days, independently separated by 1, 2, 3, 4 or 5 days. In some embodiments, the CTLA-4-containing protein is abatacept. In some embodiments, the IL-2 protein is aldesleukin.

In certain embodiments, the CTLA4-containing protein is administered to the subject weekly on day 1 of week 1 (in other words, if the first administration of the CTLA4-containing protein is administered to the subject on day 1, subsequent administrations of the CTLA4-containing protein occur at day 8, day 15, day 22 etc.) and the IL-2 protein is administered to the subject on, or beginning on, day 1 of every other week, beginning with week 3 (in other words, day 15, day 29, day 53 etc). In certain embodiments, IL-2 protein is administered to the subject on day 1 of an IL-2 protein administration week for a series of consecutive days, e.g., daily for two consecutive days, daily for three consecutive days, daily for four consecutive days or daily for five consecutive days. In certain embodiments, IL-2 protein is administered to the subject on day 1 of an IL-2 protein administration week for a series of non-consecutive days, such as 2, 3, 4, 5, 6 or 7 non-consecutive days, independently separated by 1, 2, 3, 4 or 5 days. In some embodiments, the CTLA-4-containing protein is abatacept. In some embodiments, the IL-2 protein is aldesleukin.

In some embodiments, according to the methods provided herein, the CTLA4-containing protein is administered to the subject weekly, e.g., as described in any of the embodiments presented herein in an amount in the range of 20-50 mg per week. In certain embodiments, according to the methods provided herein, the CTLA4-containing protein is administered to the subject weekly, e.g., as described in any of the embodiments presented herein in an amount in the range of 20 mg per week. In certain embodiments, according to the methods provided herein, the CTLA4-containing protein is administered to the subject weekly, e.g., as described in any of the embodiments presented herein in an amount in the range of 25 mg per week. In certain embodiments, according to the methods provided herein, the CTLA4-containing protein is administered to the subject weekly, e.g., as described in any of the embodiments presented herein in an amount in the range of 30 mg per week. In certain embodiments, according to the methods provided herein, the CTLA4-containing protein is administered to the subject weekly, e.g., as described in any of the embodiments presented herein in an amount in the range of 35 mg per week. In certain embodiments, according to the methods provided herein, the CTLA4-containing protein is administered to the subject weekly, e.g., as described in any of the embodiments presented herein in an amount in the range of 40 mg per week. In certain embodiments, according to the methods provided herein, the CTLA4-containing protein is administered to the subject weekly, e.g., as described in any of the embodiments presented herein in an amount in the range of 45 mg per week. In certain embodiments, according to the methods provided herein, the CTLA4-containing protein is administered to the subject weekly, e.g., as described in any of the embodiments presented herein in an amount in the range of 50 mg per week. In certain embodiments, the CTLA4-containing protein is abatacept. In some embodiments, the IL-2 protein, e.g., aldesleukin, administered in conjunction with methods comprising weekly administration of the CTLA4-containing protein, e.g., abatacept, is administered in an amount in the range of 10,000-3,000,000 units. In some such embodiments, the IL-2 protein, e.g., aldesleukin, is administered in an amount in the range of 500,000-3,000,000 units. In some such embodiments, the IL-2 protein, e.g. aldesleukin, is administered in an amount in the range of 500,000-2,000,000 units. In some embodiments, the IL-2 protein, e.g., aldesleukin, is administered in an amount of 1,000,000 units.

In one aspect, presented herein is a method of treating a disease or disorder in a subject in need thereof, for example, a disease or disorder described herein, e.g., a neurodegenerative or neuroinflammatory disease or disorder, comprising administering to the subject:
i) abatacept; and
ii) aldesleukin;
wherein the method mitigates one or more symptoms associated with the neurodegenerative or neuroinflammatory disease or disorder in the treated subject.

In certain embodiments, the abatacept is administered by injection or infusion. In particular embodiments, the abatacept is administered subcutaneously. In particular embodiments, the abatacept is administered intravenously. In certain embodiments, the aldesleukin is administered by injection or infusion. In particular embodiments, the aldesleukin is administered subcutaneously. In particular embodiments, the aldesleukin is administered intravenously. In certain embodiments, the abatacept and the aldesleukin are administered by injection or infusion. In particular embodiments, the abatacept and the aldesleukin are administered subcutaneously. In particular embodiments, the abatacept and the aldesleukin are administered intravenously.

In certain embodiments, the CTLA-4-containing protein, e.g., abatacept, is administered to the subject once every two weeks. In particular embodiments, the CTLA-4-containing protein, e.g., abatacept, is administered to the subject subcutaneously once every two weeks. In certain embodiments, the CTLA-4-containing protein, e.g., abatacept, is administered to the subject once every two weeks for 15 weeks. In particular embodiments, the CTLA-4-containing protein, e.g., abatacept, is administered to the subject subcutaneously once every two weeks for 15 weeks.

In certain embodiments, the IL-2 protein, e.g., aldesleukin, is administered to the subject once daily for three consecutive days. In particular embodiments, the IL-2 protein, e.g., aldesleukin, is administered to the subject subcutaneously once daily for three consecutive days.

In certain embodiments, the CTLA-4-containing protein, e.g., abatacept, is administered to the subject once every two weeks and the IL-2 protein, e.g., aldesleukin, is administered to the subject once daily for three consecutive days. In certain embodiments, the CTLA-4-containing protein, e.g., abatacept, is administered to the subject subcutaneously once every two weeks and the IL-2 protein, e.g., aldesleukin, is administered to the subject subcutaneously once daily for three consecutive days.

In certain embodiments, the CTLA-4-containing protein, e.g., abatacept, is administered to the subject once every two weeks and the IL-2 protein, e.g., aldesleukin, is administered to the subject once daily for three consecutive days beginning on the day the CTLA-4-containing protein, e.g., abatacept, is administered. In certain embodiments, the CTLA-4-containing protein, e.g., abatacept, is administered to the subject subcutaneously once every two weeks and the IL-2 protein, e.g., aldesleukin, is administered to the subject subcutaneously once daily for three consecutive days, beginning on the day the CTLA-4-containing protein, e.g., abatacept, is administered to the subject.

In certain embodiments, the CTLA-4-containing protein, e.g., abatacept, is administered to the subject once every two weeks for fifteen weeks and the IL-2 protein, e.g., aldesleukin, administration to the subject begins on week three and the IL-2 protein is administered to the subject once daily for three consecutive days beginning on the day the CTLA-4-containing protein, e.g., abatacept, is administered to the subject. In certain embodiments, the CTLA-4-containing protein, e.g., abatacept, is administered to the subject subcutaneously once every two weeks for fifteen weeks and the IL-2 protein, e.g., aldesleukin, administration to the subject begins on week three and the IL-2 protein is administered to the subject subcutaneously once daily for three consecutive days beginning on the day the CTLA-4-containing protein, e.g., abatacept, is administered.

In certain embodiments, about 5 mg to about 200 mg, about 10 mg to about 200 mg, about 15 mg to about 200 mg, about 20 mg to about 200 mg, about 25 mg to about 200 mg, about 50 to about 200 mg, about 50 mg to about 175 mg, about 50 mg to about 150 mg or about 50 mg to about 125 mg of the CTLA-4-containing protein, e.g., abatacept, is administered to the subject. In a particular embodiment, the CTLA-4-containing protein, e.g., abatacept, is subcutaneously administered to the subject.

In a particular embodiment, about 50 mg of the CTLA-4-containing protein, e.g., abatacept, is administered to the subject. In a specific embodiment, about 50 mg of the CTLA-4-containing protein, e.g., abatacept, in a 0.4 mL volume is administered to the subject. In a particular embodiment, the CTLA-4-containing protein, e.g., abatacept, is subcutaneously administered to the subject.

In a particular embodiment, about 87.5 mg of the CTLA-4-containing protein, e.g., abatacept, is administered to the subject. In a specific embodiment, about 87.5 mg of the CTLA-4-containing protein, e.g., abatacept, in a 0.7 mL volume is administered to the subject. In a particular embodiment, the CTLA-4-containing protein, e.g., abatacept, is subcutaneously administered to the subject.

In a particular embodiment, about 125 mg of the CTLA-4-containing protein, e.g., abatacept, is administered to the subject. In a specific embodiment, about 125 mg of the CTLA-4-containing protein, e.g., abatacept, in a 1.0 mL volume is administered to the subject. In a particular embodiment, the CTLA-4-containing protein, e.g., abatacept, is subcutaneously administered to the subject.

In a particular embodiment, about 1×10⁴ to about 1×10⁷, about 5×10⁴ to about 1×10⁷, about 1×10⁵ to about 1×10⁷, about 5×10⁵ to about 1×10⁷, 5×10⁵ to about 5×10⁶, 5×10⁵ to about 4×10⁶, 5×10⁵ to about 3×10⁶, 5×10⁵ to about 2×10⁶, about 5×10⁵ to about 1×10⁶ units IL-2 protein, e.g., aldesleukin, is administered to the subject. In a particular embodiment, the IL-2 protein, e.g., aldesleukin, is subcutaneously administered to the subject.

In a specific embodiment, about 500,000 units to 3,000,000 units of the IL-2 protein, e.g., aldesleukin, is administered to the subject, for example, is subcutaneously administered to the subject. In a specific embodiment, about 500,000 units to 2,000,000 units of the IL-2 protein, e.g., aldesleukin, is administered to the subject, for example, is subcutaneously administered to the subject. In a specific embodiment, about 500,000 units to 1,000,000 units of the IL-2 protein, e.g., aldesleukin, is administered to the subject, for example, is subcutaneously administered to the subject.

In some embodiments, according to the methods provided herein, the abatacept is administered in an amount in the range of 20-200 mg. In some embodiments, the abatacept is administered in an amount in the range of 25-200 mg. In some embodiments, the abatacept is administered in an amount in the range of 50-200 mg. In some embodiments, the abatacept is administered in an amount in the range of 50-175 mg. In some embodiments, the abatacept is administered in an amount in the range of 50-150 mg. In some embodiments, the abatacept is administered in an amount in the range of 50-125 mg. In some embodiments, the abatacept is administered in a 50 mg amount. In some embodiments, the abatacept is administered in an 87.5 mg amount. In some embodiments, the abatacept is administered in a 125 mg amount. In some embodiments, the abatacept is subcutaneously administered in a 0.1-2.0 mL volume. In some embodiments, the abatacept is subcutaneously administered in a 0.4 mL volume. In some embodiments, the abatacept is subcutaneously administered in a 0.7 mL volume. In some embodiments, the abatacept is subcutaneously administered in a 1.0 mL volume. In some embodiments, the abatacept is subcutaneously administered in a 50 mg amount in a 0.4 mL volume. In some embodiments, the abatacept is subcutaneously administered in an 87.5 mg amount in a 0.7 mL volume. In some embodiments, the abatacept is subcutaneously administered in a 125 mg amount in a 1.0 mL volume. In some embodiments, the aldesleukin is administered in an amount in the range of 10,000-3,000,000 units. In some embodiments, the aldesleukin is administered in an amount in the range of 500,000-3,000,000 units. In some embodiments, the aldesleukin is administered in an amount in the range of 500,000-2,000,000 units. In some embodiments, the aldesleukin is administered in an amount of 1,000,000 units. In some embodiments, the aldesleukin is administered subcutaneously.

In some embodiments, according to the methods provided herein, the abatacept is administered to the subject in an amount in the range of 20-50 mg per dosing cycle, for multiple dosing cycles. In some embodiments, the abatacept alone is administered to the subject in the absence of IL-2 in a dosing cycle of the multiple dosing cycles. In some embodiments, the abatacept is administered in combination with IL-2 in a dosing cycle of the multiple dosing cycles. In some embodiments, the multiple dosing cycles comprise (i) dosing cycles in which the abatacept alone is administered to the subject in the absence of IL-2 in each dosing cycle; and (ii) dosing cycles in which the abatacept is administered in combination with IL-2 in each dosing cycle. In certain embodiments, the multiple dosing cycles comprise (i) dosing cycles in which the abatacept alone is administered to the subject in the absence of IL-2 in each dosing cycle; and (ii) dosing cycles in which the abatacept is administered in combination with IL-2 in each dosing cycle, wherein the dosing cycles in (i) and (ii) alternate in the multiple dosing cycles (for example, a dosing cycle in which the abatacept alone is administered is followed by a dosing cycle in which the abatacept is administered in combination with IL-2, which in turn is followed by a dosing in which the abatacept alone is administered followed by a dosing cycle in which the abatacept is administered in combination with IL-2). Each of the dosing cycles in the multiple dosing cycles can be, for instance, 1 week. In certain embodiments, the IL-2 protein is aldesleukin.

In certain embodiments, according to the methods provided herein, the abatacept is administered to the subject in an amount in the range of 20-50 mg per dosing cycle, e.g., 20 mg, 25 mg, 30 mg, 35 mg, 40 mg, 45 mg or 50 mg per dosing cycle, for multiple dosing cycles, wherein the multiple dosing cycles comprise 1-week dosing cycles according to the following regimen:
cycle 1: abatacept alone, in the absence of IL-2;
cycle 2: abatacept alone, in the absence of IL-2;
cycle 3: abatacept in combination with IL-2;
cycle 4: abatacept alone, in the absence of IL-2;
   wherein dosing cycles 3 and 4 are repeated in subsequent cycles, e.g., for a total of 6, 8, 10, 12, 16, 18, 20, or more cycles. In certain embodiments, the IL-2 protein is aldesleukin.

In some embodiments, according to the methods provided herein, the abatacept is administered to the subject once every two weeks. In some embodiments, the abatacept is administered intravenously to the subject once every two weeks. In some embodiments, the abatacept is administered subcutaneously to the subject once every two weeks. In some embodiments, the abatacept is administered to the subject once every two weeks for 10-20 weeks. For example, in some embodiments, the abatacept is administered to the subject once every two weeks for 12 weeks, for 15 weeks, or for 18 weeks. In some embodiments, the abatacept is administered once every two weeks for 15 weeks. In some embodiments, the abatacept is administered to the subject once daily for 2-5 consecutive days. For example, in some embodiments, the abatacept is administered to the subject once daily for 2, 3, 4 or 5 consecutive days. In some embodiments, the abatacept is administered to the subject once daily for three consecutive days. In some embodiments, the abatacept is administered intravenously to the subject once daily for 2-5 consecutive days. In some embodiments, the abatacept is administered subcutaneously to the subject once daily for 2-5 consecutive days. For example, in some embodiments, the abatacept is administered subcutaneously to the subject once daily for 2, 3, 4 or 5 consecutive days. In some embodiments, the abatacept is administered subcutaneously to the subject once daily for three consecutive days.

In some embodiments, according to the methods provided herein, the aldesleukin is administered to the subject once daily for 2-5 consecutive days. For example, in some embodiments, the aldesleukin is administered to the subject once daily for 2, 3, 4 or 5 consecutive days. In some embodiments, the aldesleukin is administered to the subject once daily for three consecutive days. In some embodiments, the aldesleukin is administered intravenously to the subject once daily for 2-5 consecutive days. In some embodiments, the aldesleukin is administered subcutaneously to the subject once daily for 2-5 consecutive days. For example, in some embodiments, the aldesleukin is administered subcutaneously to the subject once daily for 2, 3, 4 or 5 consecutive days. In some embodiments, the aldesleukin is administered subcutaneously to the subject once daily for three consecutive days.

In some embodiments, according to the methods provided herein, the abatacept is administered once every two weeks, and the aldesleukin is administered once daily for 2-5 consecutive days, such as for three consecutive days, beginning on the day the abatacept is administered. In some embodiments, the abatacept and the aldesleukin are administered subcutaneously. In some embodiments, the abatacept is administered once every two weeks for 10-20 weeks, such as fifteen weeks, aldesleukin administration begins on week three; and once aldesleukin administration begins, the aldesleukin is administered once daily for 2-5 consecutive days, such as for three consecutive days, beginning on the day the abatacept is administered. In some embodiments, the abatacept and the aldesleukin are administered subcutaneously.

In some embodiments, according to the methods provided herein, the abatacept is administered to the subject one or more times during a dosing cycle, for example 1-10 times during a dosing cycle, such as 1, 2, 3, 4, 5, 6, 7, 8, 9 or 10 times during a dosing cycle. In some embodiments, the abatacept is administered to the subject as a single administration on day 1 of the dosing cycle. In some embodiments, the abatacept is administered to the subject daily for a series of consecutive days, beginning on day 1 of the dosing cycle. In some embodiments, the abatacept is administered to the subject daily for two consecutive days, beginning on day 1 of the dosing cycle. In some embodiments, the abatacept is administered to the subject daily for three consecutive days, beginning on day 1 of the dosing cycle. In some embodiments, the abatacept is administered to the subject daily for four consecutive days, beginning on day 1 of the dosing cycle. In some embodiments, the abatacept is administered to the subject daily for five consecutive days, beginning on day 1 of the dosing cycle. In some embodiments, the abatacept is administered to the subject for a series of non-consecutive days, beginning on day 1 of the dosing cycle, for example, administered to the subject for a series of non-consecutive days, such as 2, 3, 4, 5, 6, 7, 8, 9 or 10 non-consecutive days, independently separated by 1, 2, 3, 4 or 5 days, beginning on day 1 of the dosing cycle.

In some embodiments, according to the methods provided herein, the aldesleukin is administered to the subject one or more times during a dosing cycle, for example 1-10 times during a dosing cycle, such as 1, 2, 3, 4, 5, 6, 7, 8, 9 or 10 times during a dosing cycle. In some embodiments, the aldesleukin is administered to the subject daily for a series of consecutive days, beginning on day 1 of the dosing cycle. In some embodiments, the aldesleukin is administered to the subject daily for two consecutive days, beginning on day 1 of the dosing cycle. In some embodiments, the aldesleukin is administered to the subject daily for three consecutive days, beginning on day 1 of the dosing cycle. In some embodiments, the aldesleukin is administered to the subject daily for four consecutive days, beginning on day 1 of the dosing cycle. In some embodiments, the aldesleukin is administered to the subject daily for five consecutive days, beginning on day 1 of the dosing cycle. In some embodiments, the aldesleukin is administered to the subject for a series of non-consecutive days, beginning on day 1 of the dosing cycle, for example, administered to the subject for a series of non-consecutive days, such as 2, 3, 4, 5, 6, 7, 8, 9 or 10 non-consecutive days, independently separated by 1, 2, 3, 4 or 5 days, beginning on day 1 of the dosing cycle.

In some embodiments, according to the methods provided herein, the abatacept is administered to the subject as a single administration on day 1 of the dosing cycle. In some embodiments, the abatacept is administered daily to the subject for 2-5 consecutive days, such as for three consecutive days, beginning on day 1 of a first dosing cycle. In some embodiments, the aldesleukin is administered daily to the subject for three consecutive days, beginning on day 1 of a first dosing cycle. In some embodiments, the aldesleukin is administered to the subject daily for three consecutive days, beginning on day 1 of the first dosing cycle. In some embodiments, the aldesleukin is administered to the subject daily for three consecutive days, beginning on day 1 of the second dosing cycle. In some embodiments, the first dosing cycle is repeated 6 times, with each repeated dosing cycle beginning 14 days after day 1 of the previous dosing cycle.

In some embodiments, according to the methods provided herein, the dosing cycle is 2-6 weeks. In some embodiments, the dosing cycle is 2 weeks. In some embodiments, the dosing cycle is 3 weeks. In some embodiments, the dosing cycle is 4 weeks. In some embodiments, the dosing cycle is 5 weeks. In some embodiments, the dosing cycle is 6 weeks. In some embodiments, the dosing cycle is repeated 1-12 times. In some embodiments, the dosing cycle is repeated 10 times. In some embodiments, the dosing cycle is repeated 8 times. In some embodiments, the dosing cycle is repeated 6 times. In some embodiments, each repeated dosing cycle begins 10-28 days after day 1 of the previous dosing cycle. For example, in some embodiments, each repeated dosing cycle begins 14 days after day 1 of the previous dosing cycle. In some embodiments, each repeated dosing cycle begins 2-6 weeks after day 1 of the previous dosing cycle. In some embodiments, each repeated dosing cycle begins 2 weeks after day 1 of the previous dosing cycle. In some embodiments, each repeated dosing cycle begins 3 weeks after day 1 of the previous dosing cycle. In some embodiments, each repeated dosing cycle begins 4 weeks after day 1 of the previous dosing cycle. In some embodiments, each repeated dosing cycle begins 5 weeks after day 1 of the previous dosing cycle. In some embodiments, each repeated dosing cycle begins 6 weeks after day 1 of the previous dosing cycle.

In one aspect, presented herein is a method of treating a disease or disorder in a subject in need thereof, for example, a disease or disorder described herein, e.g., a neurodegenerative or neuroinflammatory disease or disorder, comprising a dosing cycle that begins on day 1 and comprises administering to the subject a formulation comprising:
i) a CTLA-4-containing protein; and
ii) an IL-2 protein;
wherein the method mitigates one or more symptoms associated with the neurodegenerative or neuroinflammatory disease or disorder in the treated subject. The formulation is administered to the subject one or more times during the dosing cycle. The dosing cycle may be repeated one or more times. There may be a period of time between the completion of one dosing cycle and the beginning of the next dosing cycle. In certain embodiments, the CTLA-4-containing protein is abatacept.

In certain embodiments, the IL-2 protein is aldesleukin. In certain embodiments, the CTLA-4-containing protein is abatacept and the IL-2 protein is aldesleukin.

For ease of description, the formulation may be referred to herein as a “CTLA-4-containing protein/IL-2 protein formulation,” or an “IL-2 protein/CTLA-4-containing protein formulation.” In instances wherein the CTLA-4-containing protein is abatacept and the IL-2 protein is aldesleukin, the formulation may be referred to herein as an “abatacept/aldesleukin formulation” or an “aldesleukin/abatacept formulation.”

The certain embodiments, the formulation is administered to the subject by injection or infusion. In specific embodiments, the formulation is administered to the subject subcutaneously. In specific embodiments, the formulation is administered to the subject intravenously.

In certain embodiments of such a method, the dosing cycle comprises administering the formulation to the subject 1-10 times.

In specific embodiments, of the method described herein, the dosing cycle comprises a single administration of the formulation to the subject on day 1 of the dosing cycle. In specific embodiments of such a method, the dosing cycle comprises administering the formulation to the subject daily for two consecutive days, beginning on day 1 of the dosing cycle. In specific embodiments of such a method, the dosing cycle comprises administering the formulation to the subject daily for three consecutive days, beginning on day 1 of the dosing cycle. In specific embodiments of such a method, the dosing cycle comprises administering the formulation to the subject daily for four consecutive days, beginning on day 1 of the dosing cycle. In specific embodiments of such a method, the dosing cycle comprises administering the formulation to the subject daily for five consecutive days, beginning on day 1 of the dosing cycle.

In specific embodiments of the methods described herein, the dosing cycle comprises administering the formulation to the subject daily for at least two non-consecutive days. In one non-limiting embodiment, for example, the formulation is first administered to the subject on day 1 and is next administered to the subject on day 3, day 4, day 5, day 6 or day 7 of the dosing cycle.

In certain embodiments of the methods described herein, the dosing cycle is repeated 1-12 times. In specific embodiments, the dosing cycle is repeated 6 times. In particular embodiments, each repeated dosing cycle begins 10-28 days after day 1 of the previous dosing cycle. In particular embodiments, each repeated dosing cycle begins 10-28 days after the completion of the previous dosing cycle. In specific embodiments, each repeated dosing cycle begins 14 days after day 1 of the previous dosing cycle. In particular embodiments, each repeated dosing cycle begins 14 days after the completion of the previous dosing cycle.

In certain embodiments of the methods described herein, the first dosing cycle comprises administering the formulation to the subject daily for three consecutive days, beginning on day 1 of the dosing cycle, and the first dosing cycle is repeated 6 times, with each repeated dosing cycle beginning 14 days after day 1 of the previous dosing cycle.

In certain embodiments of the methods described herein, the dosing cycle comprises administering to the subject a CTLA-4-containing protein/IL-2 protein formulation comprising about 5 mg to about 125 mg CTLA-4-containing protein and about 3×10⁴ to about 3×10⁷ units IL-2 protein. In certain embodiments of the methods described herein, the dosing cycle comprises administering to the subject an abatacept/aldesleukin formulation comprising about 5 mg to about 125 mg abatacept and about 3×10⁴ to about 3×10⁷ units aldesleukin.

In certain embodiments of the methods described herein, the dosing cycle comprises administering to the subject a formulation comprising about 8.75 mg to about 87.5 mg abatacept and about 3×10⁴ to about 3×10⁷ units aldesleukin.

In certain embodiments of the methods described herein, the dosing cycle comprises administering to the subject a formulation comprising about 29.17 mg abatacept and about 1×10⁵ units aldesleukin. In certain embodiments of the methods described herein, the dosing cycle comprises administering to the subject a formulation comprising about 29.17 mg abatacept and about 1×10⁶ units aldesleukin. In certain embodiments of the methods described herein, the dosing cycle comprises administering to the subject a formulation comprising about 29.17 mg abatacept and about 1×10⁷ units aldesleukin.

In certain embodiments of the methods described herein, the dosing cycle comprises administering to the subject a formulation comprising about 5 mg to about 50 mg abatacept and about 3×10⁴ to about 3×10⁷ units aldesleukin.

In certain embodiments of the methods described herein, the dosing cycle comprises administering to the subject a formulation comprising about 16.67 mg abatacept and about 1×10⁵ units aldesleukin. In certain embodiments of the methods described herein, the dosing cycle comprises administering to the subject a formulation comprising about 16.67 mg abatacept and about 1×10⁶ units aldesleukin. In certain embodiments of the methods described herein, the dosing cycle comprises administering to the subject a formulation comprising about 16.67 mg abatacept and about 1×10⁷ units aldesleukin.

In certain embodiments of the methods described herein, the dosing cycle comprises administering to the subject a formulation comprising about 12.5 mg to about 125 mg abatacept and about 3×10⁴ to about 3×10⁷ units aldesleukin.

In certain embodiments of the methods described herein, the dosing cycle comprises administering to the subject a formulation comprising about 41.67 mg abatacept and about 1×10⁵ units aldesleukin. In certain embodiments of the methods described herein, the dosing cycle comprises administering to the subject a formulation comprising about 41.67 mg abatacept and about 1×10⁶ units aldesleukin. In certain embodiments of the methods described herein, the dosing cycle comprises administering to the subject a formulation comprising about 41.67 mg abatacept and about 1×10⁷ units aldesleukin.

In certain embodiments of the methods described herein, the dosing cycle comprises administering the formulation to the subject daily for three consecutive days, beginning on day 1 of the dosing cycle, wherein the formulation comprises about 29.17 mg abatacept and about 1×10⁶ units aldesleukin. In specific embodiments, the dosing cycle is repeated 6 times, with each repeated dosing cycle beginning 14 days after day 1 of the previous dosing cycle.

In certain embodiments of the methods described herein, a total of 50 mg abatacept and 3×10⁵ units aldesleukin are administered to the subject per dosing cycle, via one or more administrations of an abatacept/aldesleukin formulation. In certain embodiments of the methods described herein, a total of 50 mg abatacept and 3×10⁵ units aldesleukin are administered to the subject per dosing cycle, via 1-10 administrations of an abatacept/aldesleukin formulation. In a particular embodiment, the dosing cycle comprises 1-10 administrations of an abatacept/aldesleukin formulation as shown at Table 1A.

In certain embodiments of the methods described herein, a total of 50 mg abatacept and 3×10⁶ units aldesleukin are administered to the subject per dosing cycle, via one or more administrations of an abatacept/aldesleukin formulation. In certain embodiments of the methods described herein, a total of 50 mg abatacept and 3×10⁶ units aldesleukin are administered to the subject per dosing cycle, via 1-10 administrations of an abatacept/aldesleukin formulation. In a particular embodiment, the dosing cycle comprises 1-10 administrations of an abatacept/aldesleukin formulation as shown at Table 1B.

In certain embodiments of the methods described herein, a total of 50 mg abatacept and 3×10⁷ units aldesleukin are administered to the subject per dosing cycle, via one or more administrations of an abatacept/aldesleukin formulation. In certain embodiments of the methods described herein, a total of 50 mg abatacept and 3×10⁷ units aldesleukin are administered to the subject per dosing cycle, via 1-10 administrations of an abatacept/aldesleukin formulation. In a particular embodiment, the dosing cycle comprises 1-10 administrations of an abatacept/aldesleukin formulation as shown at Table 1C.

In certain embodiments of the methods described herein, a total of 87.5 mg abatacept and 3×10⁵ units aldesleukin are administered to the subject per dosing cycle, via one or more administrations of an abatacept/aldesleukin formulation. In certain embodiments of the methods described herein, a total of 87.5 mg abatacept and 3×10⁵ units aldesleukin are administered to the subject per dosing cycle, via 1-10 administrations of an abatacept/aldesleukin formulation. In a particular embodiment, the dosing cycle comprises 1-10 administrations of an abatacept/aldesleukin formulation as shown at Table 2A.

In certain embodiments of the methods described herein, a total of 87.5 mg abatacept and 3×10⁶ units aldesleukin are administered to the subject per dosing cycle, via one or more administrations of an abatacept/aldesleukin formulation. In certain embodiments of the methods described herein, a total of 87.5 mg abatacept and 3×10⁶ units aldesleukin are administered to the subject per dosing cycle, via 1-10 administrations of an abatacept/aldesleukin formulation. In a particular embodiment, the dosing cycle comprises 1-10 administrations of an abatacept/aldesleukin formulation as shown at Table 2B.

In certain embodiments of the methods described herein, a total of 87.5 mg abatacept and 3×10⁷ units aldesleukin are administered to the subject per dosing cycle, via one or more administrations of an abatacept/aldesleukin formulation. In certain embodiments of the methods described herein, a total of 87.5 mg abatacept and 3×10⁷ units aldesleukin are administered to the subject per dosing cycle, via 1-10 administrations of an abatacept/aldesleukin formulation. In a particular embodiment, the dosing cycle comprises 1-10 administrations of an abatacept/aldesleukin formulation as shown at Table 2C.

In certain embodiments of the methods described herein, a total of 125 mg abatacept and 3×10⁵ units aldesleukin are administered to the subject per dosing cycle, via one or more administrations of an abatacept/aldesleukin formulation. In certain embodiments of the methods described herein, a total of 125 mg abatacept and 3×10⁵ units aldesleukin are administered to the subject per dosing cycle, via 1-10 administrations of an abatacept/aldesleukin formulation. In a particular embodiment, the dosing cycle comprises 1-10 administrations of an abatacept/aldesleukin formulation as shown at Table 3A.

In certain embodiments of the methods described herein, a total of 125 mg abatacept and 3×10⁶ units aldesleukin are administered to the subject per dosing cycle, via one or more administrations of an abatacept/aldesleukin formulation. In certain embodiments of the methods described herein, a total of 125 mg abatacept and 3×10⁶ units aldesleukin are administered to the subject per dosing cycle, via one or more administrations of an abatacept/aldesleukin formulation. In a particular embodiment, the dosing cycle comprises 1-10 administrations of an abatacept/aldesleukin formulation as shown at Table 3B.

In certain embodiments of the methods described herein, a total of 125 mg abatacept and 3×10⁷ units aldesleukin are administered to the subject per dosing cycle, via one or more administrations of an abatacept/aldesleukin formulation. In certain embodiments of the methods described herein, a total of 125 mg abatacept and 3×10⁷ units aldesleukin are administered to the subject per dosing cycle, via one or more administrations of an abatacept/aldesleukin formulation. In a particular embodiment, the dosing cycle comprises 1-10 administrations of an abatacept/aldesleukin formulation as shown at Table 3C.

TABLE 1
(50 mg abatacept/dosing cycle. A = 3 × 10⁵ units aldesleukin/dosing cycle; B = 3 × 10⁶
units aldesleukin/dosing cycle; C = 3 × 10⁷ units aldesleukin/dosing cycle; #/dosing
cycle = number of administrations of the formulation (number of doses) per dosing cycle)

| | #/dosing cycle | | | | | | | | | |
|---|---|---|---|---|---|---|---|---|---|---|
| | 1 | 2 | 3 | 4 | 5 | 6 | 7 | 8 | 9 | 10 |
| A | 50 | 25 | 16.67 | 15 | 10 | 8.33 | 7.14 | 6.25 | 5.56 | 5 |
| | 3 × 10⁵ | 1.5 × 10⁵ | 1 × 10⁵ | 7.5 × 10⁴ | 6 × 10⁴ | 5 × 10⁴ | 4.3 × 10⁴ | 3.75 × 10⁴ | 3.33 × 10⁴ | 3 × 10⁴ |
| B | 50 | 25 | 16.67 | 15 | 10 | 8.33 | 7.14 | 6.25 | 5.56 | 5 |
| | 3 × 10⁶ | 1.5 × 10⁶ | 1 × 10⁶ | 7.5 × 10⁵ | 6 × 10⁵ | 5 × 10⁵ | 4.3 × 10⁵ | 3.75 × 10⁵ | 3.33 × 10⁵ | 3 × 10⁵ |
| C | 50 | 25 | 16.67 | 15 | 10 | 8.33 | 7.14 | 6.25 | 5.56 | 5 |
| | 3 × 10⁷ | 1.5 × 10⁷ | 1 × 10⁷ | 7.5 × 10⁶ | 6 × 10⁶ | 5 × 10⁶ | 4.3 × 10⁶ | 3.75 × 10⁶ | 3.33 × 10⁶ | 3 × 10⁶ |

TABLE 2
(87.5 mg abatacept/dosing cycle. A = 3 × 10⁵ units aldesleukin/dosing cycle;
B = 3 × 10⁶ units aldesleukin/dosing cycle; C = 3 × 10⁷ units aldesleukin/dosing
cycle; #/dosing cycle = number of administrations of the formulation (number of doses) per dosing cycle)

| | #/dosing cycle | | | | | | | | | |
|---|---|---|---|---|---|---|---|---|---|---|
| | 1 | 2 | 3 | 4 | 5 | 6 | 7 | 8 | 9 | 10 |
| A | 87.5 | 43.75 | 29.17 | 21.88 | 17.5 | 14.58 | 12.5 | 10.94 | 9.72 | 8.75 |
| | 3 × 10⁵ | 1.5 × 10⁵ | 1 × 10⁵ | 7.5 × 10⁴ | 6 × 10⁴ | 5 × 10⁴ | 4.3 × 10⁴ | 3.75 × 10⁴ | 3.33 × 10⁴ | 3 × 10⁴ |
| B | 87.5 | 43.75 | 29.17 | 21.88 | 17.5 | 14.58 | 12.5 | 10.94 | 9.72 | 8.75 |
| | 3 × 10⁶ | 1.5 × 10⁶ | 1 × 10⁶ | 7.5 × 10⁵ | 6 × 10⁵ | 5 × 10⁵ | 4.3 × 10⁵ | 3.75 × 10⁵ | 3.33 × 10⁵ | 3 × 10⁵ |
| C | 87.5 | 43.75 | 29.17 | 21.88 | 17.5 | 14.58 | 12.5 | 10.94 | 9.72 | 8.75 |
| | 3 × 10⁷ | 1.5 × 10⁷ | 1 × 107 | 7.5 × 10⁶ | 6 × 10⁶ | 5 × 10⁶ | 4.3 × 10⁶ | 3.75 × 10⁶ | 3.33 × 10⁶ | 3 × 10⁶ |

TABLE 3
(125 mg abatacept/dosing cycle. A = 3 × 10⁵ units aldesleukin/dosing cycle; B = 3 × 10⁶
units aldesleukin/dosing cycle; C = 3 × 10⁷ units aldesleukin/dosing
cycle; #/dosing cycle = number of administrations of the formulation (number of doses) per dosing cycle)

| | #/dosing cycle | | | | | | | | | |
|---|---|---|---|---|---|---|---|---|---|---|
| | 1 | 2 | 3 | 4 | 5 | 6 | 7 | 8 | 9 | 10 |
| A | 125 | 62.5 | 41.67 | 31.25 | 25 | 20.83 | 17.86 | 15.63 | 13.89 | 12.5 |
| | 3 × 10⁵ | 1.5 × 10⁵ | 1 × 10⁵ | 7.5 × 10⁴ | 6 × 10⁴ | 5 × 10⁴ | 4.3 × 10⁴ | 3.75 × 10⁴ | 3.33 × 10⁴ | 3 × 10⁴ |
| B | 125 | 62.5 | 41.67 | 31.25 | 25 | 20.83 | 17.86 | 15.63 | 13.89 | 12.5 |
| | 3 × 10⁶ | 1.5 × 10⁶ | 1 × 10⁶ | 7.5 × 10⁵ | 6 × 10⁵ | 5 × 10⁵ | 4.3 × 10⁵ | 3.75 × 10⁵ | 3.33 × 10⁵ | 3 × 10⁵ |
| C | 125 | 62.5 | 41.67 | 31.25 | 25 | 20.83 | 17.86 | 15.63 | 13.89 | 12.5 |
| | 3 × 10⁷ | 1.5 × 10⁷ | 1 × 10⁷ | 7.5 × 10⁶ | 6 × 10⁶ | 5 × 10⁶ | 4.3 × 10⁶ | 3.75 × 10⁶ | 3.33 × 10⁶ | 3 × 10⁶ |

In certain embodiments, the methods described herein further comprise administering a CTLA-4-containing protein formulation, e.g., an abatacept formulation, to the subject prior to the first administration to the subject of the CTLA-4-containing/L-2 protein formulation, e.g., the abatacept/aldesleukin formulation. In certain embodiments, the methods described herein further comprise administering a CTLA-4-containing protein formulation, e.g., an abatacept formulation, to the subject 14 days prior to day 1 of the first dosing cycle, that is, 14 days prior to the first administration to the subject of the CTLA-4-containing/IL-2 protein formulation, e.g., the abatacept/aldesleukin formulation.

In specific embodiments, the CTLA-4-containing protein formulation comprises 50 mg to 125 mg CTLA-4-containing protein, for example, 50 mg CTLA-4-containing protein, 87.5 mg CTLA-4-containing protein or 125 mg CTLA-4-containing protein. In specific embodiments, the abatacept formulation comprises 50 mg to 125 mg abatacept, for example, 50 mg abatacept, 87.5 mg abatacept or 125 mg abatacept. In certain embodiments, the CTLA-4-containing protein formulation, e.g., abatacept formulation, is administered to the subject by injection or infusion. In certain embodiments, the CTLA-4-containing protein formulation, e.g., abatacept formulation, is administered to the subject subcutaneously or intravenously.

In some embodiments, according to the methods provided herein, the dosing cycle comprises administering the formulation to the subject 1-10 times. In some embodiments, the dosing cycle comprises a single administration of the formulation to the subject on day 1 of the dosing cycle. In some embodiments, the dosing cycle comprises administering the formulation to the subject daily for two consecutive days, beginning on day 1 of the dosing cycle. In some embodiments, the dosing cycle comprises administering the formulation to the subject daily for three consecutive days, beginning on day 1 of the dosing cycle. In some embodiments, the dosing cycle comprises administering the formulation to the subject daily for four consecutive days, beginning on day 1 of the dosing cycle. In some embodiments, the dosing cycle comprises administering the formulation to the subject daily for five consecutive days, beginning on day 1 of the dosing cycle. In some embodiments, the dosing cycle comprises administering the formulation to the subject daily for six consecutive days, beginning on day 1 of the dosing cycle. In some embodiments, the dosing cycle comprises administering the formulation to the subject daily for seven consecutive days, beginning on day 1 of the dosing cycle. In some embodiments, the dosing cycle is repeated 6 times, with each repeated dosing cycle beginning 14 days after day 1 of the previous dosing cycle. In some embodiments, the first dosing cycle comprises administering the formulation to the subject daily for 2-5 consecutive days, such as for three consecutive days, beginning on day 1 of the dosing cycle, and the first dosing cycle is repeated 6 times, with each repeated dosing cycle beginning 14 days after day 1 of the previous dosing cycle.

In some embodiments, according to the methods provided herein, the method further comprises administering an abatacept formulation to the subject 14 days prior to day 1 of the first dosing cycle, wherein the abatacept formulation comprises abatacept. In some embodiments, the abatacept formulation comprises 50 mg to 125 mg abatacept. In some embodiments, the abatacept formulation comprises 87.5 mg abatacept. In some embodiments, the abatacept is subcutaneously administered in a 0.1-2.0 mL volume. In some embodiments, the abatacept is subcutaneously administered in a 0.4 mL volume. In some embodiments, the abatacept is subcutaneously administered in a 0.7 mL volume. In some embodiments, the abatacept is subcutaneously administered in a 1.0 mL volume. In some embodiments, the abatacept is subcutaneously administered in a 50 mg amount in a 0.4 mL volume. In some embodiments, the abatacept formulation is administered by injection or infusion. In some embodiments, the abatacept formulation is administered subcutaneously. In some embodiments, the abatacept formulation is administered intravenously. In some embodiments, the abatacept is subcutaneously administered in an 87.5 mg amount in a 0.7 mL volume. In some embodiments, the abatacept is subcutaneously administered in a 125 mg amount in a 1.0 mL volume.

In certain aspects, the methods of treatment provided herein comprise administering a pharmaceutical composition described herein to a subject in need of treatment.

In some embodiments, the subject is diagnosed with or is suspected of having a disorder associated with Treg dysfunction. In some embodiments, the subject is diagnosed with or is suspected of having a disorder associated with Treg deficiency. In some embodiments, the subject is diagnosed with or is suspected of having a condition driven by a T cell response.

In some embodiments the subject is diagnosed with or is suspected of having a neurodegenerative disease. In some embodiments, the subject is diagnosed with or is suspected of having Alzheimer's disease, Amyotrophic Lateral Sclerosis, Huntington's disease, Parkinson's disease, or frontotemporal dementia.

In some embodiments, the subject is diagnosed with or is suspected of having a disorder that would benefit from downregulation of the immune system.

In some embodiments, the subject is diagnosed with or suspected of having an autoimmune disease. The autoimmune disease may be, for example, systemic sclerosis (scleroderma), polymyositis, ulcerative colitis, inflammatory bowel disease, Crohn's disease, celiac disease, multiple sclerosis (MS), rheumatoid arthritis (RA), Type I diabetes, psoriasis, dermatomyositis, systemic lupus erythematosus, cutaneous lupus, myasthenia gravis, autoimmune nephropathy, autoimmune hemolytic anemia, autoimmune cytopenia autoimmune hepatitis, autoimmune uveitis, alopecia, thyroiditis or pemphigus.

In some embodiments, the subject is diagnosed with or suspected of having heart failure or ischemic cardiomyopathy. In some embodiments, the subject is diagnosed with or suspected of having graft-versus-host disease, e.g., after undergoing organ transplantation (such as a kidney transplantation or a liver transplantation), or after undergoing stem cell transplantation (such as hematopoietic stem cell transplantation).

In some embodiments, the subject is diagnosed with or suspected of having neuroinflammation. Neuroinflammation may be associated, for example, with stroke, acute disseminated encephalomyelitis (ADEM), acute optic neuritis, transverse myelitis, neuromyelitis optica (NMO), epilepsy, traumatic brain injury, spinal cord injury, encephalitis central nervous system (CNS) vasculitis, neurosarcoidosis, autoimmune or post-infectious encephalitis, or chronic meningitis.

In some embodiments, the subject is diagnosed with or suspected of having cardo-inflammation, e.g., cardio-inflammation associated with atherosclerosis, myocardial infarction, ischemic cardiomyopathy, with heart failure.

In some embodiments, the subject is diagnosed with or suspected of having chronic inflammatory demyelinating polyradiculoneuropathy (CIDP). In some embodiments, the subject is diagnosed with or suspected of having acute inflammatory demyelinating polyneuropathy (AIDP). In some embodiments, the subject is diagnosed with or suspected of having Guillain-Barre syndrome (GBS).

In some embodiments, the subject has had a stroke.

In some embodiments, the subject is diagnosed with or suspected of having cancer, e.g., a blood cancer.

In some embodiments, the subject is diagnosed with or suspected of having asthma.

In some embodiments, the subject is diagnosed with or suspected of having eczema.

In some embodiments, the subject is diagnosed with or suspected of having a disorder associated with overactivation of the immune system.

In some embodiments, the subject is diagnosed with or suspected of having Tregopathy. The Tregopathy may be caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

5.3.1. Methods of Determining Treatment Effect

The effect of a method of treatment provided herein may be assessed by monitoring clinical signs and symptoms of the disease to be treated.

The efficacy of a method of treatment described herein may be assessed at about 4 weeks, about 8 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, at about 2-3 months, 3-4 months, 4-5 months, 5-6 months, 6-7 months, 7-8 months, 8-9 months, about 9-10 months, about 10-11 months, about 11-12 months, about 12-18 months, about 18-24 months, about 1-2 years, about 2-3 years, about 3-4 years, about 4-5 years, about 5-6 years, about 6-7 years, about 7-8 years, about 8-9 years, or about 9-10 years after initiation of treatment in accordance with the method described herein.

In some embodiments, method of treatment provided herein results in a change in the mini-mental state examination (MMSE) score compared to baseline. In the context of an assessment of the effect of a method of treatment, the term “baseline” refers to a measurement pre-treatment. The MMSE score measures overall Alzheimer's disease symptoms. In some embodiments, the MMSE score increases in a subject treated in accordance with a method provided herein compared to baseline, indicating an improvement of symptoms. In other embodiments, the MMSE score remains unchanged in a subject treated in accordance with a method provided herein compared to baseline.

In some embodiments, method of treatment provided herein results in a change in the Appel ALS score compared to baseline. In the context of an assessment of the effect of a method of treatment, the term “baseline” refers to a measurement pre-treatment. The Appel ALS score measures overall progression of disability or altered function. In some embodiments, the Appel ALS score decreases in a subject treated in accordance with a method provided herein compared to baseline, indicating an improvement of symptoms. In other embodiments, the Appel ALS score remains unchanged ins a subject treated in accordance with a method provided herein compared to baseline.

In some embodiments, a method of treatment provided herein results in a change in the Amyotrophic Lateral Sclerosis Functional Rating Scale-revised (ALSFRS-R) score compared to baseline. The ALSFRS-R score assesses the progression of disability or altered function. In some embodiments, the ALSFRS-R score increases in a subject treated in accordance with a method provided herein compared to baseline, indicating an improvement of symptoms. In other embodiments, the Appel ALSFRS-R score remains unchanged in a subject treated in accordance with a method provided herein compared to baseline.

In some embodiments, a method of treatment provided herein results in a change in forced vital capacity (FVC; strength of muscles used with expiration) compared to baseline, where the highest number is the strongest measurement. In some embodiments, FVC increases in a subject treated in accordance with a method provided herein compared to baseline. In other embodiments, FVC remains unchanged in a subject treated in accordance with a method provided herein compared to baseline.

In some embodiments, a method of treatment provided herein results in a change in Maximum Inspiratory Pressure (MIP; strength of muscles used with inspiration) compared where the highest number is the strongest measurement. In some embodiments, MIP increases in a subject treated in accordance with a method provided herein compared to baseline. In other embodiments, MIP remains unchanged in a subject treated in accordance with a method provided herein compared to baseline.

In some embodiments, a method of treatment provided herein results in a change in Neuropsychiatric Inventory Questionnaire (NPI-Q) compared to baseline. The NPI-Q provides symptom Severity and Distress ratings for each symptom reported, and total Severity and Distress scores reflecting the sum of individual domain scores. In some embodiments, the NPI-Q score decreases in a subject treated in accordance with a method provided herein compared to baseline. In other embodiments, NPI-Q score remains unchanged in a subject treated in accordance with a method provided herein compared to baseline.

In some embodiments, a method of treatment provided herein results in a decrease in the frequency of GI symptoms, anaphylaxis or seizures compared to baseline.

In some embodiments, a method of treatment provided herein results in a change in a change in CSF amyloid and/or CSF tau protein (CSF-tau) compared to baseline. In some embodiments, the levels of CSF amyloid and/or CSF tau protein decreases in a subject treated in accordance with a method provided herein compared to baseline. In other embodiments, the levels of CSF amyloid and/or CSF tau protein remains unchanged in a subject treated in accordance with a method provided herein compared to baseline.

In some embodiments, a method of treatment provided herein results in a change in Clinical Dementia Rating (CDR) compared to baseline. The CDR rates memory, orientation, judgment and problem-solving, community affairs, home and hobbies, and personal care, and a global rating is then generated, ranging from 0-no impairment to 3-severe impairment. In some embodiments, the CDR decreases in a subject treated in accordance with the methods provided herein compared to baseline. In other embodiments, the CDR remains unchanged in a subject treated in accordance with a method provided herein compared to baseline.

In some embodiments, a method of treatment provided herein results in a change in Alzheimer's Disease Assessment Scale (ADAS)-cog13 score compared to baseline. ADAS-cog tests cognitive performance and has an upper limit is 85 (poor performance) and lower limit is zero (best performance). In some embodiments, the ADAS-cog13 score decreases in a subject treated in accordance with a method provided herein compared to baseline. In other embodiments, the ADAS-cog13 score remains unchanged in a subject treated in accordance with a method provided herein.

### 5.4 Compositions

In one aspect, presented herein is a pharmaceutical composition comprising one or more doses of a CTLA-4-containing protein and an IL-2 protein (“CTLA-4-containing protein/IL-2 protein doses”). In certain embodiments, presented herein is a pharmaceutical composition comprising one or more doses of abatacept and aldesleukin (“abatacept/aldesleukin doses”).

In certain embodiments, presented herein is a pharmaceutical composition comprising one or more abatacept/aldesleukin doses, wherein an abatacept/aldesleukin dose comprises 5 mg to 125 mg abatacept and 3×10⁴ to 3×10⁷ units aldesleukin.

In particular embodiments, presented herein is a pharmaceutical composition comprising one or more abatacept/aldesleukin doses, wherein an abatacept/aldesleukin dose comprises 8.75 to 87.5 mg abatacept and 3×10⁴ to 3×10⁷ units aldesleukin. In specific embodiments, presented herein is a pharmaceutical composition comprising one or more abatacept/aldesleukin doses, wherein an abatacept/aldesleukin dose comprises 29.17 mg abatacept and 1×10⁵ units aldesleukin, 1×10⁶ units aldesleukin or 1×10⁷ units aldesleukin. In specific embodiments, presented herein is a pharmaceutical composition comprising one or more abatacept/aldesleukin doses, wherein an abatacept/aldesleukin dose comprises 29.17 mg abatacept and 1×10⁶ units aldesleukin.

In particular embodiments, presented herein is a pharmaceutical composition comprising one or more abatacept/aldesleukin doses, wherein an abatacept/aldesleukin dose comprises 5 mg to 50 mg abatacept and 3×10⁴ to 3×10⁷ units aldesleukin. In specific embodiments, presented herein is a pharmaceutical composition comprising one or more abatacept/aldesleukin doses, wherein an abatacept/aldesleukin dose comprises 16.67 mg abatacept and 1×10⁵ units aldesleukin, 1×10⁶ units aldesleukin or 1×10⁷ units aldesleukin. In specific embodiments, presented herein is a pharmaceutical composition comprising one or more abatacept/aldesleukin doses, wherein an abatacept/aldesleukin dose comprises 16.67 mg abatacept and 1×10⁶ units aldesleukin.

In particular embodiments, presented herein is a pharmaceutical composition comprising one or more abatacept/aldesleukin doses, wherein an abatacept/aldesleukin dose comprises 12.5 mg to 125 mg abatacept and 3×10⁴ to 3×10⁷ units aldesleukin. In specific embodiments, presented herein is a pharmaceutical composition comprising one or more abatacept/aldesleukin doses, wherein an abatacept/aldesleukin dose comprises 41.67 mg abatacept and 1×10⁵ units aldesleukin, 1×10⁶ units aldesleukin or 1×10⁷ units aldesleukin. In specific embodiments, presented herein is a pharmaceutical composition comprising one or more abatacept/aldesleukin doses, wherein an abatacept/aldesleukin dose comprises 41.67 mg abatacept and 1×10⁶1 units aldesleukin.

In certain embodiments, a pharmaceutical composition presented herein comprises one or more abatacept/aldesleukin doses as shown at Table 4. The shaded numbers correspond to the amount of abatacept per dose (in mg) and the unshaded numbers correspond to the amount of aldesleukin per dose (in units). Each pair of shaded (top) and unshaded (bottom) values corresponds to the amount of abatacept and aldesleukin in a particular dose. For example, in the upper left of the table, 50 (shaded, top) and 3×10⁵ (unshaded, bottom) refers to a 50 mg abatacept/3×10⁵ units aldesleukin dose.

TABLE 4
abatacept/aldesleukin doses.

| | | | | | | | | | |
|---|---|---|---|---|---|---|---|---|---|
| 50 | 25 | 16.67 | 15 | 10 | 8.33 | 7.14 | 6.25 | 5.56 | 5 |
| 3 × 10⁵ | 1.5 × 10⁵ | 1 × 10⁵ | 7.5 × 10⁴ | 6 × 10⁴ | 5 × 10⁴ | 4.3 × 10⁴ | 3.75 × 10⁴ | 3.33 × 10⁴ | 3 × 10⁴ |
| 50 | 25 | 16.67 | 15 | 10 | 8.33 | 7.14 | 6.25 | 5.56 | 5 |
| 3 × 10⁶ | 1.5 × 10⁶ | 1 × 10⁶ | 7.5 × 10⁵ | 6 × 10⁵ | 5 × 10⁵ | 4.3 × 10⁵ | 3.75 × 10⁵ | 3.33 × 10⁵ | 3 × 10⁵ |
| 50 | 25 | 16.67 | 15 | 10 | 8.33 | 7.14 | 6.25 | 5.56 | 5 |
| 3 × 10⁷ | 1.5 × 10⁷ | 1 × 10⁷ | 7.5 × 10⁶ | 6 × 10⁶ | 5 × 10⁶ | 4.3 × 10⁶ | 3.75 × 10⁶ | 3.33 × 10⁶ | 3 × 10⁶ |
| 87.5 | 43.75 | 29.17 | 21.88 | 17.5 | 14.58 | 12.5 | 10.94 | 9.72 | 8.75 |
| 3 × 10⁵ | 1.5 × 10⁵ | 1 × 10⁵ | 7.5 × 10⁴ | 6 × 10⁴ | 5 × 10⁴ | 4.3 × 10⁴ | 3.75 × 10⁴ | 3.33 × 10⁴ | 3 × 10⁴ |
| 87.5 | 43.75 | 29.17 | 21.88 | 17.5 | 14.58 | 12.5 | 10.94 | 9.72 | 8.75 |
| 3 × 10⁶ | 1.5 × 10⁶ | 1 × 10⁶ | 7.5 × 10⁵ | 6 × 10⁵ | 5 × 10⁵ | 4.3 × 10⁵ | 3.75 × 10⁵ | 3.33 × 10⁵ | 3 × 10⁵ |
| 87.5 | 43.75 | 29.17 | 21.88 | 17.5 | 14.58 | 12.5 | 10.94 | 9.72 | 8.75 |
| 3 × 10⁷ | 1.5 × 10⁷ | 1 × 10⁷ | 7.5 × 10⁶ | 6 × 10⁶ | 5 × 10⁶ | 4.3 × 10⁶ | 3.75 × 10⁶ | 3.33 × 10⁶ | 3 × 10⁶ |
| 125 | 62.5 | 41.67 | 31.25 | 25 | 20.83 | 17.86 | 15.63 | 13.89 | 12.5 |
| 3 × 10⁵ | 1.5 × 10⁵ | 1 × 10⁵ | 7.5 × 10⁴ | 6 × 10⁴ | 5 × 10⁴ | 4.3 × 10⁴ | 3.75 × 10⁴ | 3.33 × 10⁴ | 3 × 10⁴ |
| 125 | 62.5 | 41.67 | 31.25 | 25 | 20.83 | 17.86 | 15.63 | 13.89 | 12.5 |
| 3 × 10⁶ | 1.5 × 10⁶ | 1 × 10⁶ | 7.5 × 10⁵ | 6 × 10⁵ | 5 × 10⁵ | 4.3 × 10⁵ | 3.75 × 10⁵ | 3.33 × 10⁵ | 3 × 10⁵ |
| 125 | 62.5 | 41.67 | 31.25 | 25 | 20.83 | 17.86 | 15.63 | 13.89 | 12.5 |
| 3 × 10⁷ | 1.5 × 10⁷ | 1 × 10⁷ | 7.5 × 10⁶ | 6 × 10⁶ | 5 × 10⁶ | 4.3 × 10⁶ | 3.75 × 10⁶ | 3.33 × 10⁶ | 3 × 10⁶ |

In certain embodiments, presented herein is a pharmaceutical composition comprising a CTLA-4-containing protein and an IL-2 protein in a mass ratio of between 270:1 to 680:1 (CTLA4-containing protein:Il-2 protein). In some embodiments, presented herein is a pharmaceutical composition comprising a CTLA-4-containing protein and an IL-2 protein in a mass ratio of between 450:1 to 500:1 (CTLA4-containing protein:Il-2 protein). In certain embodiments, the mass ratio is 450:1, 455:1, 460:1, 465:1, 470:1, 475:1, 477:1, 480:1, 485:1, 490:1, 495:1, or 500:1 (CTLA4-containing protein:Il-2 protein). In certain embodiments, the mass ratio is 480:1 (CTLA4-containing protein:Il-2 protein).

In some embodiments, presented herein is a pharmaceutical composition comprising abatacept and aldesleukin in a mass ratio of between 270:1 to 680:1 (abatacept:aldesleukin). In some embodiments, presented herein is a pharmaceutical composition comprising abatacept and aldesleukin in a mass ratio of between 450:1 to 500:1 (abatacept:aldesleukin). In certain embodiments, the mass ratio is 450:1, 455:1, 460:1, 465:1, 470:1, 475:1, 477:1, 480:1, 485:1, 490:1, 495:1, or 500:1 (abatacept:aldesleukin). In certain embodiments, the mass ratio is 480:1 (abatacept:aldesleukin). It will be understood that the standard quantitative measure for IL-2 is the International Unit (IU), which is based not on mass of protein but on activity in a biological assay such as that as established by the World Health Organization 1st International Standard for Interleukin-2 (human). However, in practice, when manufacture of an IL-2 product is standardized, a conversion between drug mass and units is routinely possible. For the PROLEUKIN product, for instance, the conversion is 18×10⁶ IU equals 1.1 mg protein.

In certain embodiments, a pharmaceutical composition comprising one or more CTLA4-containing protein/IL-2 protein doses described herein is present in lyophilized form, for example, is present as a lyophilized powder or lyophilized cake. In certain embodiments, a pharmaceutical composition comprising one or more abatacept/aldesleukin doses described herein is present in lyophilized form, for example, is present as a lyophilized powder or lyophilized cake.

In certain embodiments, a pharmaceutical composition comprising one or more CTLA4-containing protein/IL-2 protein doses described herein is a solution, for example, an aqueous solution. In specific embodiments, the one or more CTLA4-containing protein/IL-2 protein doses are present in the pharmaceutical composition at a concentration of 1 CTLA4-containing protein/IL-2 protein dose/0.4 ml, 1 CTLA4-containing protein/IL-2 protein dose/0.7 ml, 1 CTLA4-containing protein/IL-2 protein dose/1.0 ml, 1 CTLA4-containing protein/IL-2 protein dose/1.5 ml or 1 CTLA4-containing protein/IL-2 protein dose/2.0 ml.

In certain embodiments, a pharmaceutical composition comprising one or more abatacept/aldesleukin doses described herein is a solution, for example, an aqueous solution. In specific embodiments, the one or more abatacept/aldesleukin doses are present in the pharmaceutical composition at a concentration of 1 abatacept/aldesleukin dose/0.4 ml, 1 abatacept/aldesleukin dose/0.7 ml, 1 abatacept/aldesleukin dose/1.0 ml, 1 abatacept/aldesleukin dose/1.5 ml or 1 abatacept/aldesleukin dose/2.0 ml.

In certain embodiments, a pharmaceutical composition comprising one or more CTLA4-containing protein/IL-2 protein doses described herein is suitable for subcutaneous administration. In certain embodiments, a pharmaceutical composition comprising one or more CTLA4-containing protein/IL-2 protein doses described herein is suitable for intravenous administration.

In certain embodiments, a pharmaceutical composition comprising one or more abatacept/aldesleukin doses described herein is suitable for subcutaneous administration. In certain embodiments, a pharmaceutical composition comprising one or more abatacept/aldesleukin doses described herein is suitable for intravenous administration.

In certain embodiments, presented herein is a pharmaceutical composition, comprising: i) a therapeutically effective amount of a CTLA4-containing protein, ii) a therapeutically effective amount of an IL-2 protein; and iii) one or more inactive ingredients comprising pharmaceutically acceptable salts, excipients, or carriers. In some embodiments, the therapeutically effective amount of the CTLA4-containing protein is in the range of 25-200 mg, such as 25-200 mg, 50-200 mg, 50-175 mg, 50-150 mg, or 50-125 mg. In some embodiments, the therapeutically effective amount of the CTLA4-containing protein is 50 mg. In some embodiments, the therapeutically effective amount of the CTLA4-containing protein is 87.5 mg. In some embodiments, the therapeutically effective amount of the CTLA4-containing protein is 125 mg. In some embodiments, the therapeutically effective amount of the IL-2 protein is in the range of 10,000-3,000,000 units, such as 500,000-3,000,000 units or 500,000-2,000,000 units. In some embodiments, the therapeutically effective amount of the IL-2 protein is 1,000,000 units. In some embodiments, the therapeutically effective amount of the IL-2 protein is 2,000,000 units. In some embodiments, the therapeutically effective amount of the IL-2 protein is 3,000,000 units. In some embodiments, the therapeutically effective amount of the IL-2 protein is 4,000,000 units. In some embodiments, the therapeutically effective amount of the IL-2 protein is 5,000,000 units.

In certain embodiments, presented herein is a pharmaceutical composition, comprising: i) a therapeutically effective amount of abatacept, ii) a therapeutically effective amount of aldesleukin; and iii) one or more inactive ingredients comprising pharmaceutically acceptable salts, excipients, or carriers. In some embodiments, the therapeutically effective amount of the abatacept is in the range of 25-200 mg, such as 25-200 mg, 50-200 mg, 50-175 mg, 50-150 mg, or 50-125 mg. In some embodiments, the therapeutically effective amount of the abatacept is 50 mg. In some embodiments, the therapeutically effective amount of the abatacept is 87.5 mg. In some embodiments, the therapeutically effective amount of the abatacept is 125 mg. In some embodiments, the therapeutically effective amount of the aldesleukin is in the range of 10,000-3,000,000 units, such as 500,000-3,000,000 units or 500,000-2,000,000 units. In some embodiments, the therapeutically effective amount of the aldesleukin is 1,000,000 units. In some embodiments, the therapeutically effective amount of aldesleukin is 2,000,000 units. In some embodiments, the therapeutically effective amount of aldesleukin is 3,000,000 units. In some embodiments, the therapeutically effective amount of aldesleukin is 4,000,000 units. In some embodiments, the therapeutically effective amount of aldesleukin is 5,000,000 units.

An effective amount, e.g., an effective amount of abatacept or aldesleukin, refers to an amount which is sufficient to result in a desired outcome. An effective amount may, for example refer to the amount, e.g., the amount of abatacept or aldesleukin, in a dose that is administered to a subject as part of a dosing regimen which results in a desired outcome. Such a dosing regimen may comprise administration of a single dose or administration of more than one dose, e.g., multiple doses. Such a dosing regimen may, for example, comprise a single dosing cycle or more than one dosing cycle, each dosing cycle of which may comprise administration of a single dose or administration of more than one dose, e.g., multiple doses.

In some embodiments, the one or more inactive ingredients included in the a pharmaceutical composition provided herein, comprises pharmaceutically acceptable salts, excipients, or carriers, selected from the group consisting of: sodium chloride, sodium dodecyl sulfate, monobasic sodium phosphate, dibasic sodium phosphate, maltose, mannitol, poloxamer, or sucrose. In some embodiments, the pharmaceutical composition is a lyophilized powder. In some embodiments, the pharmaceutical composition is a solution. For example, in some embodiments, the solution is an aqueous solution.

In certain embodiments CTLA4-containing protein/IL-2 protein pharmaceutical composition, such as an abatacept/aldesleukin pharmaceutical composition, as provided herein is suitable for self-administration (e.g., cutaneous administration) by a subject, for example with a pre-filled syringe, an injection device (e.g., an INJECT-EASE™ or a GENJECT™ device), an infusion pump (e.g. an Accu-Chek™ infusion pump), an injector pen (e.g., a GENPEN™ injector pen), a needleless device (e.g., a MEDDECTOR™ or BIOJECTOR™ needless device), or an autoinjector (e.g., a ClickJect™ autoinjector).

In some embodiments, the CTLA4-containing protein/IL-2 protein, e.g., abatacept/aldesleukin, pharmaceutical composition is administered with an autoinjector, which may for example, be a delivery pen with a mechanism for automation. Such an autoinjector may use any mechanism for automation known in the art (e.g., a spring loaded needle or a liquefied gas, such as liquefied hydrofluoroalkane). One of skill in the art will appreciate that in some embodiments, in using an autoinjector, a subject may actuate drug delivery without activating a push-button (e.g., solely through the application of pressure on the injection site). In some embodiments, an autoinjector used for the administration of a pharmaceutical composition disclosed herein may be a device that wholly or partially replaces the activities involved in drug delivery from a standard syringe. As a non-limiting example, these activities may include removing the protective syringe cap, inserting a needle into the skin of the patient, injecting medication, removing the needle, shielding the needle, and preventing reuse of the device.

In some embodiments, the self-administration device, for example, the injection device (e.g., autoinjector, such as autoinjector pen) is mechanical. In some embodiments, the injection device (e.g., autoinjector, such as autoinjector pen) is electronic. Such injection devices may be provided separate from a pharmaceutical composition or prefilled with the pharmaceutical composition. In some embodiments, the injection device is prefilled. In some embodiments, the device is empty and can be filled using cassette or cartridges.

In some embodiments, a device suitable for self-administration, e.g., subcutaneous administration, of a pharmaceutical composition disclosed herein is provided in a single-use container (e.g., a single-use vial, ampoule, syringe, or autoinjector). In some embodiments, a single-use container can be disposable. In some embodiments, an injection device suitable for self-administration, e.g., subcutaneous administration, of a pharmaceutical composition disclosed herein can be provided prefilled with a pharmaceutical composition held in a reservoir within the device, and once the reservoir is emptied of the pharmaceutical composition, the entire device can be discarded.

In some embodiments, a device suitable for self-administration, e.g., subcutaneous administration, of a pharmaceutical composition disclosed herein is reusable. As a non-limiting example, in some embodiments, a reusable autoinjector delivery device may utilize a replaceable cartridge that contains a pharmaceutical composition, and once the pharmaceutical composition within the cartridge has been administered and the cartridge is empty or no longer needed, the cartridge can be discarded and replaced with a new cartridge that contains a pharmaceutical composition.

In certain embodiments, any pen and/or autoinjector injection device known in the art may be used for the subcutaneous delivery of a pharmaceutical composition disclosed herein.

### 5.5 Additional Therapies

In some embodiments, a subject treated in accordance with the method of treatment described herein further receives one or more additional therapy or additional therapies known in the art for treating diseases such as neurodegenerative and neuroinflammatory diseases.

In some embodiments, the subjected treated in accordance with the methods described herein receives one or more additional therapies are for the treatment of Alzheimer's. Addition therapies for the treatment of Alzheimer's may include acetylcholinesterase inhibitors (e.g., donepezil (Aricept®), galantamine (Razadyne®), or rivastigmine (Exelon®)) or NMDA receptor antagonists (e.g., Memantine (Akatinol®, Axura®, Ebixa®/Abixa®, Memox® and Namenda®). Additional therapies may also include anti-inflammatory agents (e.g., nonsteroidal anti-inflammatory drugs (NSAID) such as ibuprofen, indomethacin, and sulindac sulfide)), neuronal death associated protein kinase (DAPK) inhibitors such as derivatives of 3-amino pyridazine, Cyclooxygenases (COX-1 and -2) inhibitors, or antioxidants such as vitamins C and E.

In some embodiments, a subject treated in accordance with the methods described herein receives one or more additional therapies for the treatment of ALS. Additional therapies for the treatment of ALS may include Riluzole (Rilutek®) or Riluzole (Rilutek®).

In some embodiments, a subject treated in accordance with the methods described herein receives one or more additional therapy, which can include, but is not limited to:
(a) a TNF alpha inhibitor (e.g., infliximab, adalimumab (Humira®), etanercept, golimumab, or certolizumab);
(b) an IL-6 inhibitor (e.g., siltuximab, tocilizumab, olokizumab, elsilimomab, clazakizumab, or sirukumab);
(c) an IL-23 inhibitor (e.g., tildrakizumab, guselkumab, or risankizumab);
(d) an IL-17 inhibitor (e.g., secukinumab, ixekizumab, or brodalumab);
(e) an IL-12/IL-23 subunit p40 inhibitor (e.g., ustekinumab or briakinumab);
(f) an IL-1 inhibitor (e.g., anakinra (Kineret®), canakinumab, or rilonacept);
(g) a C3-targeted complement inhibitor (e.g., pegcetacoplan);
(h) a C5-targeted complement inhibitor (e.g., ravulizumab or eculizumab);
(i) a JAK inhibitor (e.g., baricitinib, tofacitinib, or upadacitinib);
(j) an anti-CD40 CD40L (e.g., toralizumab, dapirolizumab pegol, or ruplizumab); or

(k) an CD14 inhibitor (e.g., IC14).

In some embodiments, a subject treated in accordance with the method of treatment described herein further receives a Treg cell therapy. A Treg cell therapy is described, for instance, in WO 2021/113685 A2, which is incorporated herein in its entirety for all purposes.

In some embodiments, a subject treated in accordance with the method of treatment described herein further receives extracellular vesicles (EVs) that are derived from ex vivo-expanded human Tregs as therapy (“Treg EV therapy”). Treg EV therapy is described, for instance, in International Application No. PCT/US2022/017990, filed Feb. 25, 2022, which is incorporated herein in its entirety for all purposes.

In one aspect, presented herein is a method of treating a disease or disorder in a subject in need thereof, for example, a disease or disorder described herein, e.g., a neurodegenerative or neuroinflammatory disease or disorder, comprising administering to the subject:
i) an IL-2 protein (e.g., aldesleukin); and
ii) an additional therapy;
wherein the method mitigates one or more symptoms associated with the neurodegenerative or neuroinflammatory disease or disorder in the treated subject, and wherein the additional therapy comprises: (a) a TNF alpha inhibitor (e.g., infliximab, adalimumab (Humira®), etanercept, golimumab, or certolizumab); (b) an IL-6 inhibitor (e.g., siltuximab, tocilizumab, olokizumab, elsilimomab, clazakizumab, or sirukumab); (c) an IL-23 inhibitor (e.g., tildrakizumab, guselkumab, or risankizumab); (d) an IL-17 inhibitor (e.g., secukinumab, ixekizumab, or brodalumab); (e) an IL-12/IL-23 subunit p40 inhibitor (e.g., ustekinumab or briakinumab); (f) an IL-1 inhibitor (e.g., anakinra (Kineret®), canakinumab, or rilonacept); (g) a C3-targeted complement inhibitor (e.g., pegcetacoplan); (h) a C5-targeted complement inhibitor (e.g., ravulizumab or eculizumab); (i) a JAK inhibitor (e.g., baricitinib, tofacitinib, or upadacitinib); (j) an anti-CD40 CD40L (e.g., toralizumab, dapirolizumab pegol, or ruplizumab); (k) an CD14 inhibitor (e.g., IC14); (1) a Treg cell therapy, e.g., as described in WO 2021/113685 A2; or (m) Treg EV therapy, e.g., as described in International Application No. PCT/US2022/017990, filed Feb. 25, 2022.

### 5.6 Additional Therapeutic Interventions

In some embodiments, the methods disclosed herein can be employed with one or more additional therapeutic interventions known in the art for treating diseases such as neurodegenerative and neuroinflammatory diseases, for example, ALS or Alzheimer's disease. As a non-limiting example, in some embodiments the additional therapeutic intervention may comprise a cognitive rehabilitation program, neurostimulation technique, or a combination thereof.

Any cognitive rehabilitation program known in the art can be used with the methods disclosed herein. Cognitive training, stimulation, and rehabilitation methods and software provided via digital devices are used in the art to improve the cognitive function in subjects with neurodegenerative and neuroinflammatory diseases, for example, Alzheimer's disease (Irazoki, E. et al., Front. Psychol. 11:648 (2020). In some embodiments, the cognitive rehabilitation program is a computer-implemented cognitive rehabilitation program. As a non-limiting example, in some embodiments the computer-implemented cognitive rehabilitation program may include: FesKits (Gaitin et al., 2012, Int. J. Geriatr. Psychiatry 28, 91-99), SOCIABLE (Barban et al., 2015, Int. J. Geriatr. Psychiatry 31, 340-348; and Danassi, 2015, Adv. Exp. Med. Biol. 821, 129-130), Brainer (Cavallo et al., 2016, Arch. Clin. Neuropsychol. 31, 868-876; Cavallo and Angilletta, 2018, J. Appl. Gerontol. 38, 1035-1044), NeuronUp (Mendoza Laiz et al., 2018, Restor. Neurol. Neurosci. 36, 207-213), and ComCog (Hwang et al., 2015, J. Phys. Ther. Sci. 27, 2921-2923).

In some embodiments, the neurostimulation technique is a non-invasive brain stimulation (NIBS). In some embodiments, the neurostimulation technique is an invasive brain stimulation (IBS). Any neurostimulation techniques known in the art can be used with the methods disclosed herein. As a non-limiting example, IBS includes deep brain stimulation (DBS), and invasive vagus nerve stimulation (VNS), and NIBS includes transcranial magnetic stimulation (TMS), transcranial direct current stimulation (tDCS), transcranial alternating current stimulation (tACS), electroconvulsive treatment (ECT), magnetic seizure therapy (MST), cranial electrostimulation (CES), and/or non-invasive VNS. In some embodiments, the neurostimulation technique is invasive vagus nerve stimulation or non-invasive VNS. As a non-limiting example, in some embodiments, the additional therapeutic intervention is implantation and use of a vagus stimulator (e.g., NeuroCybernetic Prosthesis, Cyberonics Inc., Houston TX.) See, e.g., Sjogren, M J et al., J Clin Psychiatry. (2002) 63(11):972-80. In some embodiments, any methods known in the art to use vagus nerve stimulation to enhance cognition in a subject may be used (e.g., by programming a pulse generator that delivers electrical signals using parameters known in the art).

### 5.7 Kits

In one aspect, presented herein is a kit, comprising, in separate containers, i) one or more doses of a formulation comprising 50 to 125 mg abatacept, and ii) one or more doses of a formulation comprising 500,000 to 3,000,000 units aldesleukin. In certain embodiments, the kit comprises one or more doses of a formulation comprising 50 mg abatacept, 87.5 mg abatacept or 125 mg abatacept. In specific embodiments, the one or more doses of abatacept are present in lyophilized form, e.g., are present as a lyophilized powder or cake. In certain embodiments, the formulation of one or more doses of abatacept is suitable for subcutaneous administration or intravenous administration. In certain embodiments, the kit comprises one or more doses of a formulation comprising 500,000 to 2,000,000 units aldesleukin or 1,000,000 units aldesleukin. In specific embodiments, the one or more doses of aldesleukin are present in lyophilized form, e.g., are present as a lyophilized powder or cake. In certain embodiments, the formulation of one or more doses of aldesleukin is suitable for subcutaneous administration or intravenous administration.

In certain embodiments, provided herein is a kit, comprising, in separate containers, i) one or more doses of a formulation comprising an amount in the range of 20-200 mg abatacept, and ii) one or more doses of a formulation comprising an amount in the range of 10,000-3,000,000 units aldesleukin. In some embodiments, the abatacept formulation comprises an amount in the range of 25-200 mg abatacept, such as 25-200 mg, 50-200 mg, 50-175 mg, 50-150 mg, or 50-125 mg abatacept. In some embodiments, the abatacept formulation comprises 50 mg abatacept. In some embodiments, the abatacept formulation comprises 87.5 mg abatacept. In some embodiments, the abatacept formulation comprises 125 mg abatacept. In some embodiments, the aldesleukin formulation comprises an amount in the range of 10,000-3,000,000 units aldesleukin, such as 500,000-3,000,000 units or 500,000-2,000,000 units aldesleukin. In some embodiments, the aldesleukin formulation comprises 1,000,000 units aldesleukin. For example, in some embodiments, the kit provided herein, comprises, in separate containers, i) one or more doses of a formulation comprising 87.5 mg abatacept, and ii) one or more doses of a formulation comprising 1,000,000 units aldesleukin.

In some embodiments, according to the kit provided herein, the abatacept formulation is an intravenous formulation. In some embodiments, the intravenous abatacept formulation is a lyophilized powder. In some embodiments, the intravenous abatacept formulation further comprises monobasic sodium phosphate. In some embodiments, the intravenous abatacept formulation further comprises sodium chloride. In some embodiments, the intravenous abatacept formulation further comprises maltose. In some embodiments, the intravenous abatacept formulation has a pH in the range of 7.2-7.8 when reconstituted in 3.5 mL of Sterile Water for Injection, USP.

In some embodiments, according to the kit provided herein, the abatacept formulation is a subcutaneous formulation. In some embodiments, the subcutaneous abatacept formulation is a solution having a pH in the range of 6.8-7.4. In some embodiments, the subcutaneous abatacept formulation further comprises dibasic sodium phosphate. In some embodiments, the subcutaneous abatacept formulation further comprises monobasic sodium phosphate. In some embodiments, the subcutaneous abatacept formulation further comprises poloxamer. In some embodiments, the subcutaneous abatacept formulation further comprises sucrose. In some embodiments, the subcutaneous abatacept formulation further comprises Sterile Water for Injection, USP. In some embodiments, the volume of the abatacept formulation is 0.1-2.0 mL. For example, in some embodiments, the volume of the abatacept formulation is 0.4 mL, 0.7 mL, or 1.0 mL.

In some embodiments, according to the kit provided herein, the aldesleukin formulation is a subcutaneous formulation. In some embodiments, the aldesleukin formulation is a lyophilized powder. In some embodiments, the aldesleukin formulation further comprises dibasic sodium phosphate. In some embodiments, the aldesleukin formulation further comprises monobasic sodium phosphate. In some embodiments, the aldesleukin formulation further comprises sodium dodecyl sulfate. In some embodiments, the aldesleukin formulation further comprises mannitol. In some embodiments, the aldesleukin formulation, when reconstituted in Sterile Water for Injection, USP, at a concentration of 18,000,000 units per 1 mL, has a pH in the range of 7.2-7.8.

In some embodiments, a kit provided herein comprises instructions for use, additional reagents (e.g., sterilized water or saline solutions for dilution of the compositions), or components, such as tubes, containers or syringes for collection of biological samples, processing of biological samples, and/or reagents for quantitating the amount of one or more surface markers in a sample (e.g., detection reagents, such as antibodies).

In some embodiments, the kits contain one or more containers containing an abatacept formulation and an aldesleukin formulation for use in the methods provided herein. The one or more containers holding the abatacept formulation may be a single-use vial or a multi-use vial. The one or more containers holding the aldesleukin formulation may be a single-use vial or a multi-use vial. In some embodiments, the article of manufacture or kit may further comprise a third container comprising a suitable diluent. In some embodiments, the kit contains instruction for use (e.g., dilution and/or administration) of the abatacept formulation and/or the aldesleukin formulation provided herein.

In some embodiments, a kit provided herein comprises multiple doses or administration units of one or more pharmaceutical together with one or more devices for application (e.g., syringe(s), injection pen(s) and/or autoinjector(s)). In some embodiments, such devices may be provided separate from a pharmaceutical composition or prefilled with the pharmaceutical composition. In some embodiments, a kit provided herein comprises one or more doses of a pharmaceutical composition and/or formulation in separate containers. In some embodiments, the containers are enclosed in an injection device or can be inserted into an injection device (e.g., disposable dose cassettes or cartridges that can be inserted into an autoinjector device for administration).

In one aspect, presented herein is a kit comprising, in one container, a pharmaceutical composition comprising one or more doses of a CTLA-4-containing protein, e.g., abatacept, and an IL-2 protein, e.g., aldesleukin (“CTLA-4-containing protein/IL-2 protein doses”). In some embodiments, the kit further comprises instructions for use, additional reagents (e.g., sterilized water or saline solutions for dilution of the compositions), or components, such as tubes, containers or syringes for collection of biological samples, processing of biological samples, reagents for quantitating the amount of one or more surface markers in a sample (e.g., detection reagents, such as antibodies), and/or one or more devices for administration (e.g., syringe(s), injection pen(s) and/or autoinjector(s)).

### 6. EXAMPLES

### 6.1 Example 1: In Vitro Results of CTLA4 IgG (Abatacept) and Interleukin-2 (Il-2) Combination

The experiments described in this Example demonstrate that the combination of CTLA4 IgG (abatacept) and IL-2 synergistically enhance the suppressive function of Tregs.

6.1.1. Impact of Ascending Dose of CTLA4 IgG (Abatacept) on M1 IL6 Protein Expression

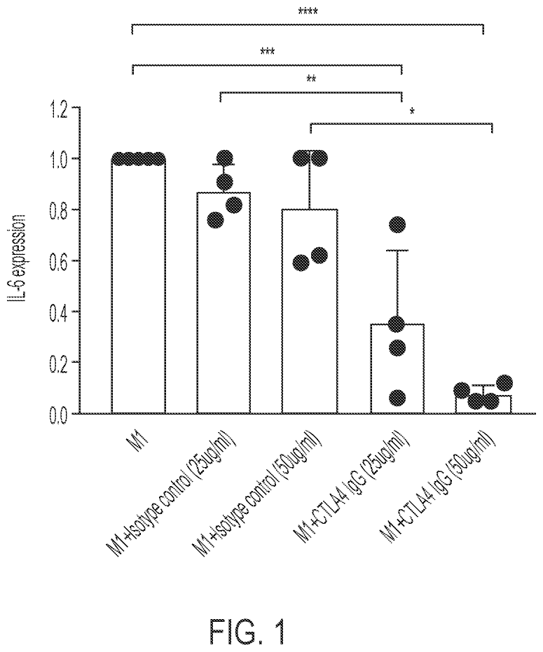

CTLA4 IgG (abatacept) or its isotype control were added to induced pluripotent stem cell (iPSC)-derived pro-inflammatory macrophages (M1) in vitro and changes in pro-inflammatory IL-6 protein expression were assayed by enzyme-linked immunoassay (ELISA). Abatacept reduced M1 IL6 protein expression in a dose-dependent manner, while the isotype control had no statistically significant effect on IL-6 expression. The results are summarized in FIG. 1.

6.1.2. Impact of Ascending Dose of Abatacept on T Responder Proliferation

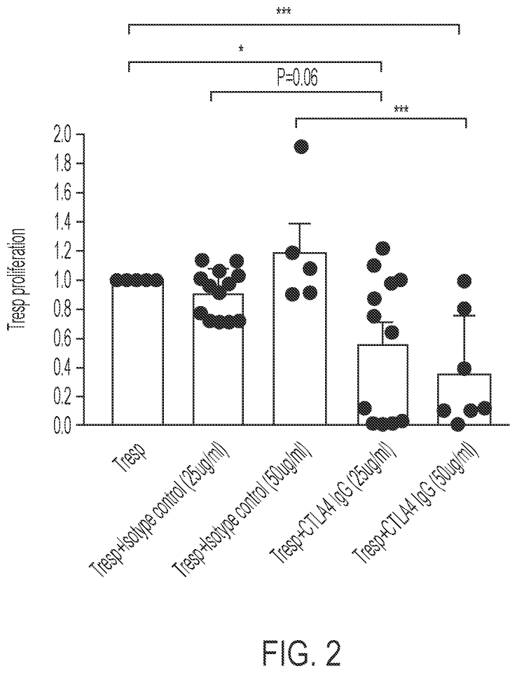

T responder cells (Tresp) were isolated from blood of Alzheimer's disease patients who had not received IL-2 therapy and placed in a 96-well plate at a density of 50,000 cells per plate. CTLA4 IgG (abatacept) or its isotype control were added to the Alzheimer's disease patient Tresp. After 5 days in culture, Tresp proliferation was assayed via thymidine incorporation. Abatacept reduced Tresp proliferation in a dose-dependent manner, while the isotype control had no statistically significant effect on Tresp proliferation. The results are summarized in FIG. 2.

6.1.3. Impact of Ascending Dose of Abatacept on the Ability of IL-2-Induced In Vivo-Expanded Alzheimer's Disease Treg to Suppress Tresp Proliferation

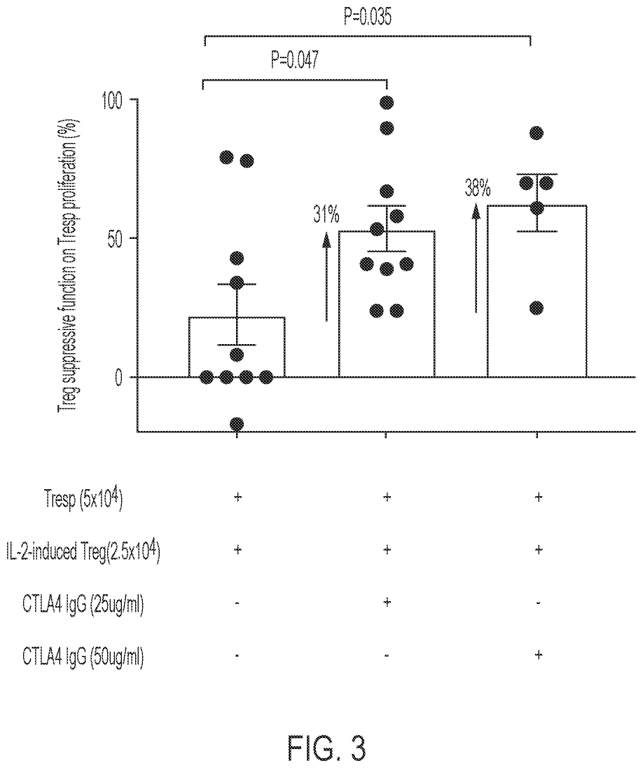

Alzheimer's disease patients received a 5-day course of IL-2 (1×10⁶ units aldesleukin) by subcutaneous injection to expand Tregs in vivo. On day 8, IL-2-induced, in vivo-expanded Tregs were isolated from patient blood samples. The 5-day course of IL-2 therapy increased the number of Tregs in the blood up to 2-fold, as measured with flow cytometry. The ability of IL-2-induced in vivo-expanded Tregs to suppress Tresp proliferation was assayed in vitro via thymidine incorporation. Addition of abatacept to IL-2-induced, in vivo expanded Tregs, enhanced the ability of the Tregs to suppress Tresp proliferation in a dose-dependent manner. The results are summarized in FIG. 3.

6.1.4. Impact of Ascending Dose of Abatacept on the Ability of IL-2-Induced In Vivo-Expanded Alzheimer's Disease Treg to Suppress M1 IL6 Production

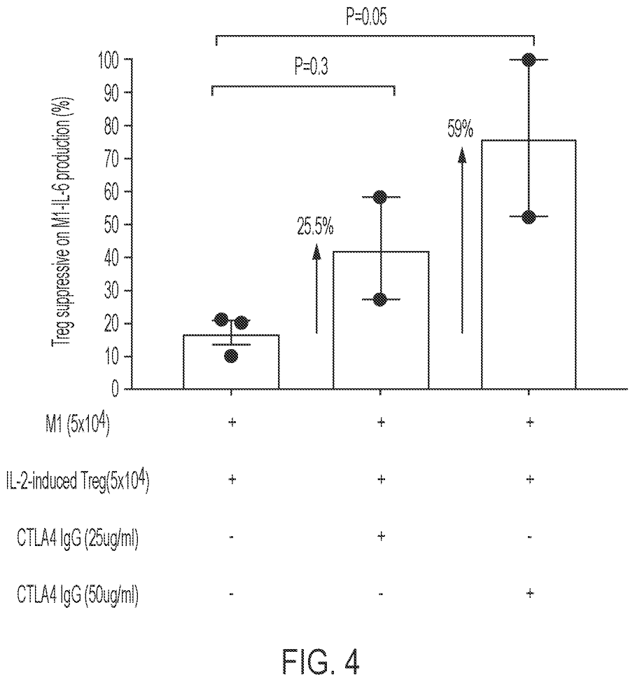

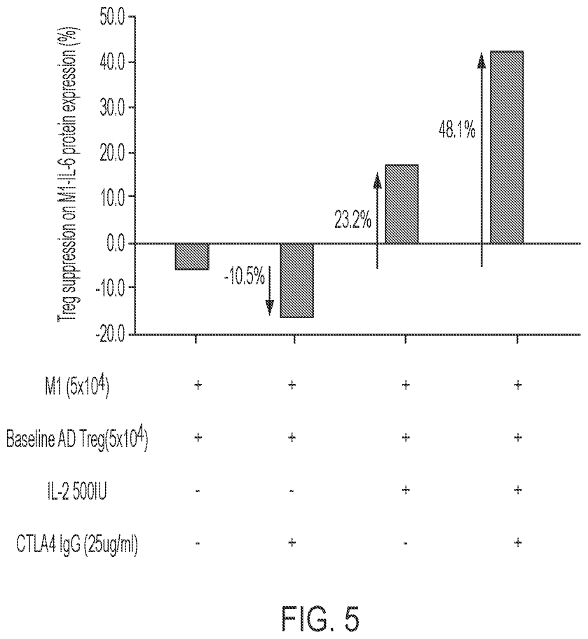

Alzheimer's disease patients received a 5-day course of IL-2 by subcutaneous injection to expand Tregs in vivo. On day 8, IL-2-induced, in vivo expanded Tregs were isolated from patient blood samples, and co-cultured with iPSC-derived pro-inflammatory M1 macrophages in vitro for 24 hours. Cultured media was collected to assess cytokine protein levels via ELISA, and the ability of Tregs to suppress pro-inflammatory M1 macrophage function was assayed. Addition of abatacept to IL-2-induced, in vivo-expanded Tregs:M1 co-culture, synergistically enhanced the ability of the Tregs to suppress M1 IL6 protein expression in a dose-dependent manner. The results are summarized in FIG. 4. The enhancement being synergistic is supported, for example, by the fact that addition of abatacept alone to co-culture actually worsens the suppressive function, whereas adding IL-2 and abatacept results in substantial enhancement of Treg suppressive function, as described in the next section and summarized in FIG. 5.

6.1.5. Impact of Abatacept and IL-2 on Alzheimer's Disease Treg Suppressive Function

Tregs were isolated from Alzheimer's disease patients who had not received IL-2 therapy and co-cultured with pro-inflammatory (M1) macrophages, as described above. Addition of IL-2 and abatacept to the Treg:M1 co-culture synergistically enhanced the ability of the Tregs to suppress pro-inflammatory M1 function as measured by IL-6 expression, while addition of abatacept alone actually decreased IL-6 expression. The results are summarized in FIG. 5.

### 6.2 Example 2: Phase I Trial Using Abatacept and Interleukin-2 (I1-2) in Patients with Alzheimer's Disease (AD)

The purpose of this study is to assess the effects of low-dose abatacept followed by IL-2 administration in patients with AD. In particular, this is a Phase I open-labeled study to assess the safety and tolerability of abatacept followed by low-dose subcutaneous IL-2 administration. Briefly, patients receive low-dose abatacept followed by IL-2 administration for a total of 4 months. Changes in inflammation markers are measured during the study period.

6.2.1. Primary Objective

To assess the safety and the tolerability of abatacept followed by IL-2 administration in AD patients, administered according to the dosage described in this protocol.

6.2.2. Secondary Objective

To investigate the immunomodulatory effects of abatacept followed by IL-2, administered in patients with AD, by comparing before, during and after treatment: (a) to monitor the change in the number and immunophenotype of Tregs; (b) to monitor the change in the suppressive activity of CD4+CD25+ FoxP3+ Tregs on T effectors proliferation; (c) to monitor the change in the level of cytokines secreted by PBMCs throughout the course of the study; and (d) to measure disease progression throughout the course of trial.

6.2.3. Study Design

This is a Phase I open-labeled uncontrolled study to assess the safety of the administration of abatacept followed by subcutaneous IL-2 administration.

Four AD patients with mild clinical dementia (MMSE between 12-25) receive a fixed low-dose of abatacept followed by IL-2 treatment for a total of 4 months.

In addition to assessing the safety and toxicity of the low-dose abatacept followed by IL-2 administration in treating the progression of AD, the goal of this phase I study is to evaluate the magnitude of Treg suppressive function enhancement in the subjects. The measurement for evaluating the enhancement of Tregs suppression is the area under the curve (AUC).

Patients receive a fixed dose of subcutaneous abatacept (87.5 mg/0.7 mL) at Day 1 of week 1. Two weeks later (Day 1 (D1) of week 3) patients receive the second dose of subcutaneous abatacept (87.5 mg/0.7 mL). In addition, patients receive subcutaneous IL-2 (1×10⁶ units/day) for 3 days (Day 1-3 (D1-3) of week 3). If this treatment regimen is tolerated, patients receive 6 further similar treatment courses of abatacept and IL-2 every two weeks.

Alternative treatment strategies may be employed. For example, in a five-day administration schedule, patients receive a fixed dose of subcutaneous abatacept (87.5 mg/0.7 mL) at Day 1 of week 1. Two weeks later (Day 1 (D1) of week 3) patients receive the second dose of subcutaneous abatacept (87.5 mg/0.7 mL). In addition, patients receive subcutaneous IL-2 (1×10⁶ units/day) for 5 days (Day 1-5 (D1-5) of week 3), with patients receiving 6 similar treatment courses of abatacept and IL-2 every two weeks. In another alternative example, in a seven-day administration schedule, patients receive a fixed dose of subcutaneous abatacept (87.5 mg/0.7 mL) at Day 1 of week 1. Two weeks later (Day 1 (D1) of week 3) patients receive the second dose of subcutaneous abatacept (87.5 mg/0.7 mL). In addition, patients receive subcutaneous IL-2 (1×10⁶ units/day) for 7 days (Day 1-7 (D1-7) of week 3), with patients receiving 6 similar treatment courses of abatacept and IL-2 every two weeks.

In another alternative example, in a five-day administration schedule, patients receive a fixed dose of subcutaneous abatacept (125 mg/0.7 mL) at Day 1 of week 1. Two weeks later (Day 1 (D1) of week 3) patients receive the second dose of subcutaneous abatacept (125 mg/0.7 mL). In addition, patients receive subcutaneous IL-2 (1×10⁶ units/day) for 5 days (Day 1-5 (D1-5) of week 3), with patients receiving 6 similar treatment courses of abatacept and IL-2 every two weeks. See Table 5.

TABLE 5
Abatacept/IL-2 Administration Schedule

| | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| | Weeks of Therapy (three-day administration schedule) | | | | | | | | | | | | | | | |
| Abatacept | D 1 | | D 1 | | D 1 | | D 1 | | D 1 | | D 1 | | D 1 | | D 1 | |
| 87.5 mg | | | | | | | | | | | | | | | | |
| IL-2 | | | D 1-3 | | D 1-3 | | D 1-3 | | D 1-3 | | D 1-3 | | D 1-3 | | D 1-3 | |
| Injection | | | | | | | | | | | | | | | | |
| 10⁶ unit | | | | | | | | | | | | | | | | |
| | Weeks of Therapy (five-day administration schedule) | | | | | | | | | | | | | | | |
| Abatacept | D 1 | | D 1 | | D 1 | | D 1 | | D 1 | | D 1 | | D 1 | | D 1 | |
| 87.5 mg | | | | | | | | | | | | | | | | |
| IL-2 | | | D 1-5 | | D 1-5 | | D 1-5 | | D 1-5 | | D 1-5 | | D 1-5 | | D 1-5 | |
| Injection | | | | | | | | | | | | | | | | |
| 10⁶ unit | | | | | | | | | | | | | | | | |
| | Weeks of Therapy (seven-day administration schedule) | | | | | | | | | | | | | | | |
| Abatacept | D 1 | | D 1 | | D 1 | | D 1 | | D 1 | | D 1 | | D 1 | | D 1 | |
| 87.5 mg | | | | | | | | | | | | | | | | |
| IL-2 | | | D 1-7 | | D 1-7 | | D 1-7 | | D 1-7 | | D 1-7 | | D 1-7 | | D 1-7 | |
| Injection | | | | | | | | | | | | | | | | |
| 10⁶ unit | | | | | | | | | | | | | | | | |
| | Weeks of Therapy (three-day administration schedule) | | | | | | | | | | | | | | | |
| Abatacept | D 1 | | D 1 | | D 1 | | D 1 | | D 1 | | D 1 | | D 1 | | D 1 | |
| 125 mg | | | | | | | | | | | | | | | | |
| IL-2 | | | D 1-3 | | D 1-3 | | D 1-3 | | D 1-3 | | D 1-3 | | D 1-3 | | D 1-3 | |
| Injection | | | | | | | | | | | | | | | | |
| 10⁶ unit | | | | | | | | | | | | | | | | |
| | Weeks of Therapy (five-day administration schedule) | | | | | | | | | | | | | | | |
| Abatacept | D 1 | | D 1 | | D 1 | | D 1 | | D 1 | | D 1 | | D 1 | | D 1 | |
| 125 mg | | | | | | | | | | | | | | | | |
| IL-2 | | | D 1-5 | | D 1-5 | | D 1-5 | | D 1-5 | | D 1-5 | | D 1-5 | | D 1-5 | |
| Injection | | | | | | | | | | | | | | | | |
| 10⁶ unit | | | | | | | | | | | | | | | | |
| | Weeks of Therapy (seven-day administration schedule) | | | | | | | | | | | | | | | |
| Abatacept | D 1 | | D 1 | | D 1 | | D 1 | | D 1 | | D 1 | | D 1 | | D 1 | |
| 125 mg | | | | | | | | | | | | | | | | |
| IL-2 | | | D 1-7 | | D 1-7 | | D 1-7 | | D 1-7 | | D 1-7 | | D 1-7 | | D 1-7 | |
| Injection | | | | | | | | | | | | | | | | |
| 10⁶ unit | | | | | | | | | | | | | | | | |

6.2.4. Drug Information

Interleukin 2. The recombinant human IL-2 used is PROLEUKIN (aldesleukin). Currently, the approved dose PROLEUKIN (aldesleukin) is 600,000 International Units/kg (0.037 mg/kg). In this study, patients receive subcutaneous IL-2 (1×10⁶ units) which is a fixed dose roughly 2.5% of an average on-label single dose infusion.

PROLEUKIN (aldesleukin) is made for injection and is a highly purified protein with a molecular weight of approximately 15,300 Daltons. The chemical name is des-alanyl-1, serine-125 human interleukin-2. PROLEUKIN differs from native IL-2 in the following ways: a) it is derived from E. coli and therefore not glycosylated; b) it has no N-terminal alanine—the codon for this amino acid was deleted during the genetic engineering procedure; c) it has substituted cysteine for serine at amino acid position 125 by site specific manipulation during the genetic engineering procedure; and d) its aggregation state is likely different from native IL-2.

PROLEUKIN is supplied as a sterile, white to off-white, lyophilized cake in single-use vials intended for intravenous (IV) or subcutaneous administration. Vials of lyophilized PROLEUKIN for injection should be protected from light.

When reconstituted with 1.2 mL Sterile Water for Injection (SWFI), USP, each mL contains 18 million IU (1.1 mg) PROLEUKIN, 50 mg mannitol, and 0.18 mg sodium dodecyl sulfate, buffered with approximately 0.17 mg monobasic and 0.89 mg dibasic sodium phosphate to a pH of 7.5 (range 7.2 to 7.8).

Under aseptic conditions under a laminar flow hood, vials of PROLEUKIN are reconstituted with 1.2 mL SWFI and samples further diluted with D₅W (Dextrose 5% in Water) to a concentration of 200 μg/mL and stored in Becton-Dickinson (B-D) plastic syringes at 2° C. to 8° C. (36° F. to 46° F.). Under these conditions, stability and sterility have been maintained for up to 14 days. Following delivery of the diluted syringes to the study participants, the syringes should also be kept under refrigeration (2° C. to 8° C.) at home before usage.

Vials of reconstituted IL-2 solution are further diluted with an appropriate volume of D₅W, to be able to give a subcutaneous dose of 1 million units per dose (subcutaneous dose should not exceed 2 mL).

The biological potency of PROLEUKIN is determined by a lymphocyte proliferation bioassay and is expressed in International Units (IU) as established by the World Health Organization 1st International Standard for human IL-2. The relationship between potency and protein mass is as follows: 18 million (18×106) IU PROLEUKIN=1.1 mg protein.

Abatacept. Abatacept is an FDA approved medication marketed as ORENCIA that has been indicated as a monotherapy or concomitantly with other anti-inflammatory drugs to modulate inflammation in autoimmune disorders.

The ORENCIA (abatacept) dose for this study is a fixed dose of 87.5 mg/0.7 mL. The dose amount for this study is consistent with the current approved, marketed ORENCIA label which lists the following approved injection doses: 50 mg/0.4 mL, 87.5 mg/0.7 mL, and 125 mg/mL of a clear to slightly opalescent, colorless to pale-yellow solution in a single-dose prefilled glass syringe.

Abatacept is a recombinant soluble fusion protein containing the extracellular domain of human cytotoxic T-lymphocyte-associated antigen 4 (CTLA-4) linked to the modified Fc (hinge, CH2, and CH3 domains) portion of human immunoglobulin G1.

6.2.5. Dosing Duration

PROLEUKIN. This study utilizes PROLEUKIN administrations for 3-day cycles for 15 weeks with breaks every other week. Approved duration of PROLEUKIN administration is 5-day cycles with breaks between for a total treatment period of 19 days. The longer total treatment duration of this study is substantiated from prior phase I studies and preclinical evidence of acceptable tolerability.

ORENCIA. This study proposes ORENCIA administration once weekly for a total of 16 weeks with breaks every other week. Approved duration of ORENCIA administration is introduced either via loading or fixed doses followed by continuous therapy on dosing schedules varying by indication. The treatment duration utilized in this study pairs with the IL-2 administration and there is no need for continuous therapy.

6.2.6. Patient Evaluation

6.2.6.1 Prior to Beginning Therapy

The following pre-treatment evaluation is completed prior to first abatacept/IL-2 injections:
(a) Baseline history and physical exam;
(b) Vital signs, Weight;
(c) Pulse oximetry;
(d) Labs: Complete blood count, PT/PTT, chemistries, liver function tests, thyroid function tests, QuantiFERON;
(e) Baseline ECG; and
(f) Baseline Research Labs: Treg, Th1, and Treg suppression.

6.2.6.2 During Therapy

The following data is obtained on Day 1 and every two weeks while receiving abatacept/IL-2 infusions:
(a) Physical exam; and
(b) Vital signs, Weight, Pulse oximetry.

The following lab tests are obtained every two weeks during abatacept/IL-2 therapy: Complete blood count, chemistries, liver function tests.

The following lab tests are obtained on Day 1 and every two weeks during abatacept/IL-2 therapy: Research Labs (Treg analysis): Treg, Th1, and Treg suppression.

6.2.6.3 Post Therapy

Once IL-2 treatment cycles are complete, the treated patient undergoes the following investigations at weeks 17 and 24:
(a) Physical exam;
(b) Vital signs, weight, and pulse oximetry; and
(c) Labs: complete blood count, chemistries, liver function tests, thyroid function tests, Treg analysis.

6.2.6.4 Disease Specific Evaluation

Cognitive status, including MMSE, is performed at baseline and weeks 5, 9, 13, 17 and 24 of the trial.

Cognitive status, including ADAS-Cog and CDR-SB, is performed at baseline, and weeks 13 and 24.

6.2.6.5 Immune Reconstitution Analysis

Depending on the availability of patient samples and reagents, immune reconstitution studies, including immunophenotyping and functional analysis, are obtained serially at the following time points before, during and after treatment.

Approximately 1-2 tablespoons (15-30 ml) of patient blood are taken, if feasible, at baseline visit and Day 1 (before abatacept treatment) of each treatment cycle during therapy, then at weeks 17 and 24.

If a patient's hemoglobin is less than 8.0 g/dL at any of the evaluation times, the amount of blood drawn for evaluation is reduced and may be obtained over more than one venipuncture, if necessary.

6.2.6.6 Management of Hypotension

Interruption of IL-2 for grade 3 or greater hypotension. Appropriate hydration fluids should be administered to maintain blood pressure. If this process fails, patients are managed according to guidelines in the intensive care unit.

6.2.6.7 Management of Respiratory Distress/Dyspnea

Patients are given supplemental oxygen and appropriate imaging studies. If not effective, patients is managed in the intensive care unit.

6.2.6.8 Management of Infection

Patients who develop a new infection while undergoing treatment with abatacept should be monitored closely and receive standard treatment. Administration of abatacept should be discontinued if a patient develops a serious infection. Prior to treating patients with abatacept, patient will be screened for tuberculosis. Should a patient test positive for tuberculosis screening, the patient should be treated in accordance with standard medical practice and excluded from the study.

6.2.6.9 Modifications of Therapy

Abatacept/IL-2 therapy is held for the following: patients with hypotension unresponsive to fluids, dyspnea or oxygen saturation <90% on 2 liters of supplemental oxygen; acute mental status changes; grade 3 ventricular or supraventricular arrhythmias; evidence of myocarditis or ischemia; bilirubin or creatinine >5 mg/dL; evidence of sepsis; or any other grade 3 or 4 poorly tolerated toxicity. The treatment may be restarted at a 50% dose reduction for all toxicities returning to grade 1 or less within 72 hours, with the exception of grade 3 or 4 ventricular arrhythmias, myocardial infarction, intubation, sepsis, coma, dialysis, or any toxicity considered to be life-threatening.

6.2.7. Inclusion Criteria for Initial Study Enrollment

Patients will be eligible for initial enrollment on this study if they meet the following criteria:
(a) Diagnosis of probable Alzheimer disease according to National Institute on Aging-Alzheimer's Association (NIA-AA) criteria 13;
(b) Male or female age 60 to 86 years;
(c) MMSE between 12-25;
(d) Total bilirubin less than or equal to 1.5 mg/dL;
(e) Alanine aminotransferase level (ALT) less than or equal to five times normal, albumin greater than or equal to 3.0 gm/dL;
(f) Serum creatinine less than 1.5 mg/dL;
(g) English language speaking;
(h) A family member or caretaker who is expected to be consistently available, administer both study drugs of abatacept and IL-2 and attend study visits throughout the study; and
(i) For AD patients with limited decision-making capacity, the legally authorized representative (LAD) should be present and consent based on the patient's best interest.

6.2.8. Exclusion Criteria for Initial Study Enrollment

Patients will be ineligible to participate if any of the following are true:
(a) Serious, active bacterial, fungal or viral infection, active or latent tuberculosis;
(b) Severe pulmonary dysfunction. FEV1 and FVC less than 40% of predicted (or 3 SD below normal) at baseline, If a pulmonary function test is clinically indicated. History of intubation for >72 hours;
(c) Severe cardiac dysfunction defined as left ventricular ejection fraction <40% if an echocardiogram is medically indicated to clarify ongoing symptoms or EKG findings; a history of non-controlled cardiac arrhythmias; history of cardiac tamponade; Unstable angina or MI in the last 3 months;
(d) Hypersensitivity or allergy to IL-2 or abatacept;
(e) History of bowel ischemia/perforation, or GI bleeding requiring surgery;
(f) History of resistant seizures, history of coma or toxic psychosis lasting >48 hours;
(g) Platelets <100,000/mm³; hematocrit (HCT)<30%;
(h) History of cancer in the past 5 years (except cutaneous Basal cell carcinoma or squamous cell carcinoma); or
(i) Hx of immunosuppressive therapy including IL-2 administration in the past 90 days.

6.2.9. Concomitant Medications

Antibiotics, antifungals, antivirals, G/GM-CSF and immune serum globulin will be permitted and instituted according to outlined standard operating procedures. Nutritional support including tube feeds and/or total parenteral nutrition is also permitted. Tylenol as needed for fever and appropriate clinical response to fever per standard operating procedures should be implemented.

6.2.10. Excluded Medications

The biologic response modifier interferon and TNFα blockers should not be given in conjunction with IL-2/abatacept treatment regimen. The continuous use of steroids should not be given in conjunction with IL-2.

6.2.11. Withdrawal of Subjects

6.2.11.1 Criteria for the Withdrawal of Subjects

Criteria for the withdrawal of subjects include:
(a) Any patient who develops irreversible or life threatening, or non-hematologic Grade 3 or 4 toxicity considered to be primary related to abatacept or IL-2;
(b) Severe, life-threatening infection;
(c) Intubation;
(d) Coma;
(e) Severe cardiac dysfunction, including evidence for ischemia, cardiac arrest, decreased LVSF (Left Ventricular Systolic Function) to less than 25%, life-threatening cardiac arrhythmia;
(f) Severe pulmonary dysfunction or severe pulmonary insufficiency requiring more than 2 liters of oxygen to maintain oxygen saturations greater than 90%;
(g) Severe renal dysfunctions with creatinine >5 mg/dL or renal dialysis; or
(h) Severe liver dysfunction with direct bilirubin >5 mg/dL or liver failure.

### 6.3 Example 3: Phase I Study Using Low-Dose IL-2 Followed by a Combination of Abatacept and Interleukin-2 (IL-2) in Two Patients with Alzheimer's Disease (AD)

It has been previously documented that Tregs immunophenotype and function are compromised in AD patients, however, dysfunctional AD Tregs can be normalized by expanding them in vitro in the presence of IL-2 (Faridar et al., Brain Commun. 2.2 (2020): fca 112).

The in vitro data described in Example 1 demonstrated a synergistic effect of IL-2 and abatacept in remodeling immunologic pathways in Alzheimer's disease; adding abatacept to IL-2-treated Tregs substantially improved the immunomodulatory function of the cells. To evaluate this finding in the clinical setting, two patients who initially received a four-month low dose IL-2 monotherapy were later administered an abatacept/IL-2 combination therapy.

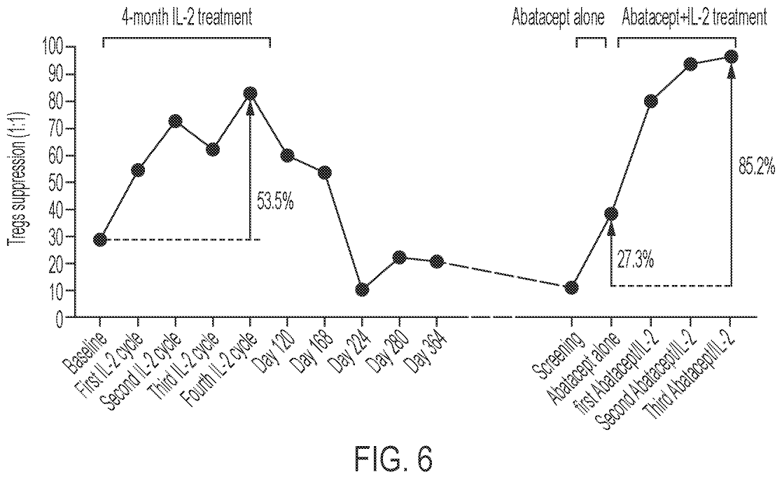

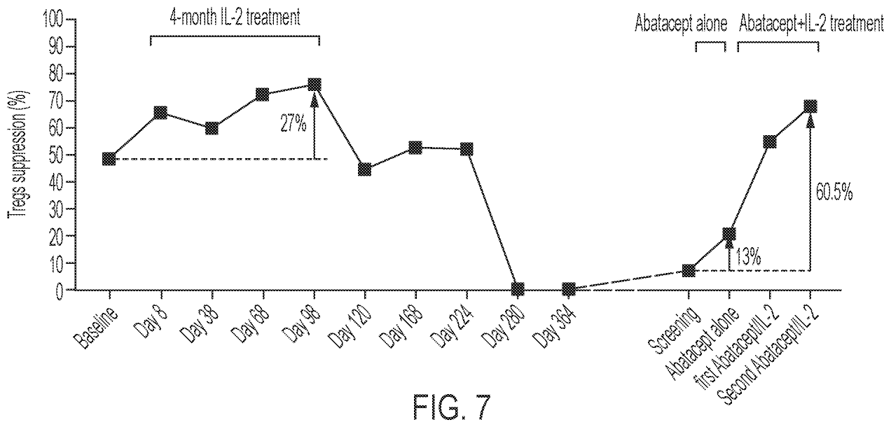

In particular, the two AD patients had received a four-month treatment of subcutaneous low dose IL-2 (1×10⁶ units/day). Patients received the low dose IL-2 for 5 days (Day 1-5 (D1-5) of week 1, week 5, week 9, and week 13, as depicted in Table 6. Tregs were isolated from patients and co-cultured with T responder (Tresp) in 1:1 ratio. IL-2 monotherapy enhanced Treg immunosuppressive function on T responder (Tresp) proliferation (Treg:Tresp ratio 1:1) (see, e.g., FIGS. 6 and 7, “4-month IL-2 monotherapy”).

TABLE 6
IL-2 Administration Schedule

| Weeks of Therapy (five-day administration schedule) | | | | | | | | | | | | | | | |
|---|---|---|---|---|---|---|---|---|---|---|---|---|---|---|---|
| 1 | 2 | 3 | 4 | 5 | 6 | 7 | 8 | 9 | 10 | 11 | 12 | 13 | 14 | 15 | 16 |

| | | | | |
|---|---|---|---|---|
| IL-2 | D | D | D | D |
| Injection | 1-5 | 1-5 | 1-5 | 1-5 |
| 10⁶ unit | | | | |

Following a period of approximately ten months after the end of the four-month low dose IL-2 monotherapy, the two patients who had initially received the monotherapy received abatacept alone, followed by low dose IL-2 and 87.5 mg abatacept administration according to the three-day administration protocol described in Table 5 (see, e.g., FIGS. 6 and 7, “Abatacept alone” and “Abatacept+IL-2 treatment”, respectively).

Treg suppressive functions on Tresp proliferation were monitored as a surrogate biomarker of response to treatment. As discussed below, IL-2 plus abatacept treatment had a synergistic effect in restoring Treg immunosuppressive function. Moreover, as also discussed below, restoration of Treg immunodulatory function amplified cognitive function of the enrolled AD patients.

6.3.1. Effect of IL-2 and Abatacept Treatment in Restoring Treg and Cognitive Function in AD Patients

Two Alzheimer's disease patients received a 4-month course of low-dose IL-2 monotherapy by subcutaneous injection to expand Tregs in vivo, as described above. IL-2 administration selectively expanded peripheral Treg populations and enhanced their immunosuppressive function. As described in Section 6.3 above, the two AD patients later received abatacept alone and combination of low-dose IL-2 and 87.5 mg abatacept according to the three-day administration protocol described in Table 5. See, e.g., FIGS. 6-8.

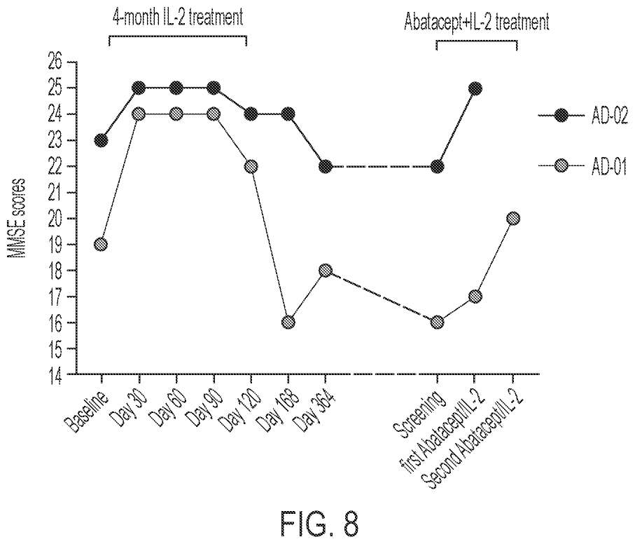

FIG. 6 illustrates that Patient AD-01 showed a 53.5% increase in Treg suppression at the fourth IL-2 monotherapy cycle, but then exhibited a rapid decrease in Treg suppression. The patient also exhibited a concomitant decline in cognitive function as measured by MMSE, shown in FIG. 8. Once patient AD-01 was administered a combination treatment of abatacept and IL-2, Treg suppressive function on Tresp proliferation increased to 85.2%, a level beyond that observed in response to IL-2 (53.5%) or abatacept alone (27.3%) (FIG. 6). Patient AD-01 also showed improved cognitive function (FIG. 8).

Similarly, as shown in FIG. 7, patient AD-02 showed a 27% increase in Treg suppression at day 98 of IL-2 monotherapy treatment, followed by a dramatic decrease in Treg suppression by day 280. Patient AD-02 exhibited a concomitant decline in cognitive function as measured by MMSE (FIG. 8). Upon initiation of abatacept/IL-2 combination treatment, the patient showed a 60.5% increase in Treg suppressive function on Tresp proliferation after the second abatacept/IL-2 administration, a level beyond that observed in response to IL-2 (27.3%) or abatacept alone (13%) (FIG. 7) and, further, exhibited a concomitant increase in cognitive function as measured by MMSE (FIG. 8).

6.3.2. Comparison of MMSE Score and Treg Suppression in Additional AD Patients on IL-2 Monotherapy Versus IL-2 Plus Abatacept Therapy

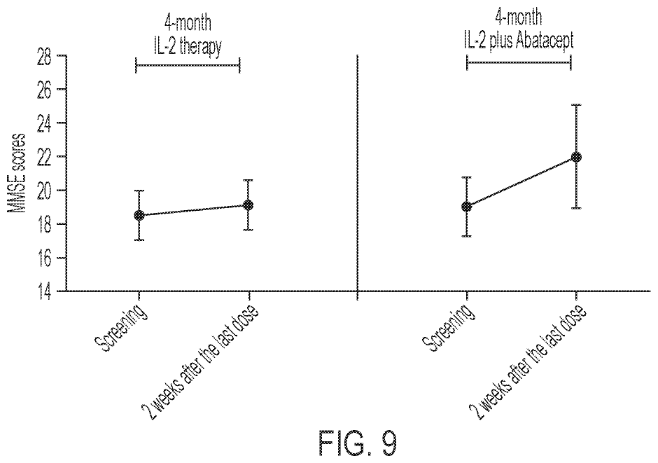

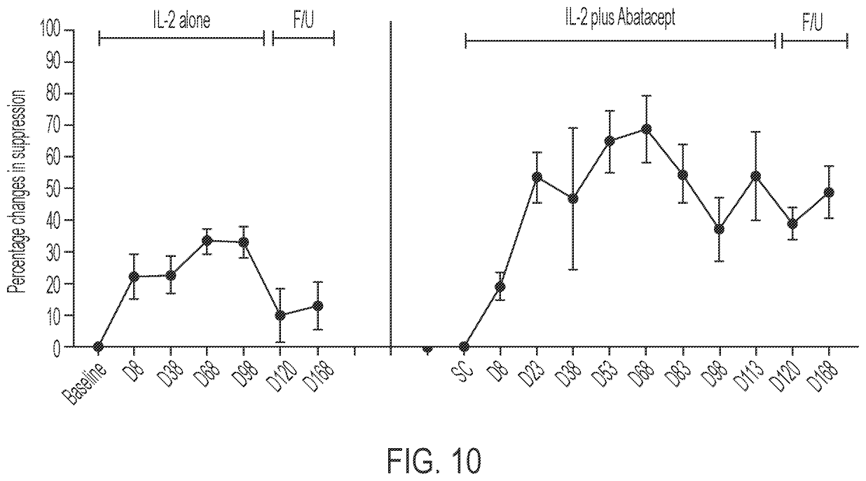

In addition to the two Alzheimer's disease patients described in the previous section, another six Alzheimer's disease patients received a 4-month course of low-dose IL-2 monotherapy by subcutaneous injection (to make eight AD patients in total). Cognitive status was evaluated with MMSE test at baseline and 2 weeks after the last cycle of IL-2 immunotherapy (monotherapy) in the 8 AD patients (FIG. 9, left panel). Treg suppressive function was also assessed in the 8 AD patients, as shown in FIG. 10 (left panel).

Three patients (patients AD-01, AD02, described above, and a third patient who had not previously received IL-2 monotherapy) received abatacept alone and combination of low-dose IL-2 and 87.5 mg abatacept according to the three-day administration protocol described in Section 6.3 and in Table 5, above. Cognitive status was monitored with MMSE changes at baseline and 2 weeks after the last cycle of IL-2 plus Abatacept immunotherapy, as shown in FIG. 9 (right panel). Treg suppressive function was evaluated in these 3 patients, as shown in FIG. 10 (right panel).

Results depicted in FIG. 9 demonstrating a beneficial effect on cognition of the IL-2/abatacept combination therapy compared to IL-2 administered alone. In particular, the results show that administration of IL-2/abatacept resulted in a 15.7% improvement in MMSE score at two weeks post treatment compared to pre-treatment screening, versus only a 3.3% increase with IL-2 administration alone. This amounts to a 4.75-fold incremental percentage improvement in MMSE score with IL-2/abatacept versus IL-2 administered alone. Results depicted in FIG. 10 show percentage changes in Treg suppressive function over pre-treatment baseline over the course of the studies. The results show an increase of Treg suppressive function in AD patients receiving IL-2/abatacept compared to patients receiving IL-2 alone that demonstrates a synergistic effect of the combination therapy on the Treg suppressive function. Further, the data demonstrate that the IL-2/abatacept treatment results in a much more successful maintenance of Treg suppressive function post-treatment than IL-2 treatment alone, which showed a substantial decline in Treg suppressive function over the same post-treatment time period.

### 6.4 Example 4: Phase I Trial Using Combination of Abatacept and Interleukin-2 (11-2) in Patients with Alzheimer's Disease

The purpose of this study is to assess the effects of IL-2 and low-dose abatacept administration in patients with AD. In particular, this is a Phase I open-labeled study to assess the safety and tolerability of IL-2 and low-dose abatacept administered subcutaneously in a single formulation. Changes in inflammation markers are measured during the study period.

6.4.1. Primary Objective

To assess the safety and the tolerability of IL-2 and low-dose abatacept in AD patients, administered in a single formulation according to the dosage described in this protocol.

6.4.2. Secondary Objective

To investigate the immunomodulatory effects of IL-2 and low-dose abatacept administered in a single formulation to patients with AD, by comparing before, during and after treatment: (a) to monitor the change in the number and immunophenotype of Tregs; (b) to monitor the change in the suppressive activity of CD4+CD25+ FoxP3+ Tregs on T effectors proliferation; (c) to monitor the change in the level of cytokines secreted by PBMCs throughout the course of the study; and (d) to measure disease progression throughout the course of trial.

6.4.3. Study Design

This is a Phase I open-labeled uncontrolled study to assess the safety of the administration of IL-2 and low-dose abatacept subcutaneously administered as a single formulation.

AD patients with mild clinical dementia (MMSE between 12-25) receive fixed low-dose of IL-2 and abatacept treatment for a total of 4 months, wherein the IL-2 and abatacept are administered in a single formulation.

In addition to assessing the safety and toxicity of the administration in treating the progression of AD, the goal of this phase I study is to evaluate the magnitude of Treg suppressive function enhancement in the subjects. The measurement for evaluating the enhancement of Tregs suppression is the area under the curve (AUC).

Patients receive a combination of 1×10⁶ units IL-2 (PROLEUKIN; aldesleukin) and 29.17 mg abatacept per day for 3 days (Day 1-3; D1-3) of week 1, delivered subcutaneously in a single formulation. If this treatment regimen is tolerated, patients receive over the next 15 weeks further similar treatment courses of the subcutaneous combination of IL-2 and abatacept every two weeks. Alternative treatment strategies may be employed. For example, patients receive a combination of 1×10⁶ units IL-2 (PROLEUKIN; aldesleukin) and 17.5 mg abatacept per day for 5 days (Day 1-5; D1-5) of week 1, delivered subcutaneously in a single formulation, followed by patients receiving over the next 15 weeks further similar treatment courses of the subcutaneous combination of IL-2 and abatacept every two weeks. In another alternative, patients receive a combination of 1×10⁶ units IL-2 (PROLEUKIN; aldesleukin) and 12.5 mg abatacept per day for 7 days (Day 1-7; D1-7) of week 1, delivered subcutaneously in a single formulation, followed by patients receiving over the next 15 weeks further similar treatment courses of the subcutaneous combination of IL-2 and abatacept every two weeks. See Table 7.

TABLE 7
Abatacept/IL-2 Combination Administration Schedule

| | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| | Weeks of Therapy (three-day administration schedule) | | | | | | | | | | | | | | | |
| Combination | D | | D | | D | | D | | D | | D | | D | | D | |
| of Abatacept | 1-3 | | 1-3 | | 1-3 | | 1-3 | | 1-3 | | 1-3 | | 1-3 | | 1-3 | |
| 29.17 mg and | | | | | | | | | | | | | | | | |
| IL-2 Injection | | | | | | | | | | | | | | | | |
| 10\hat{ }6 unit | | | | | | | | | | | | | | | | |
| | Weeks of Therapy (five-day administration schedule) | | | | | | | | | | | | | | | |
| Combination | D | | D | | D | | D | | D | | D | | D | | D | |
| of Abatacept | 1-5 | | 1-5 | | 1-5 | | 1-5 | | 1-5 | | 1-5 | | 1-5 | | 1-5 | |
| 17.5 mg and | | | | | | | | | | | | | | | | |
| IL-2 Injection | | | | | | | | | | | | | | | | |
| 10\hat{ }6 unit | | | | | | | | | | | | | | | | |
| | Weeks of Therapy (seven-day administration schedule) | | | | | | | | | | | | | | | |
| Combination | D | | D | | D | | D | | D | | D | | D | | D | |
| of Abatacept | 1-7 | | 1-7 | | 1-7 | | 1-7 | | 1-7 | | 1-7 | | 1-7 | | 1-7 | |
| 12.5 mg and | | | | | | | | | | | | | | | | |
| IL-2 Injection | | | | | | | | | | | | | | | | |
| 10\hat{ }6 unit | | | | | | | | | | | | | | | | |

Patient evaluation criteria, inclusion and exclusion criteria, and information on concomitant and excluded medications and withdrawal of subjects are the same for this study as for the study presented in Example 1.

### 6.5 Example 5: Stability of an IL-2/Abatacept Combination Formulation

This example demonstrates that an aqueous formulation comprising IL-2 and abatacept remains stable under standard test conditions (STC) for up to 24 hours at room temperature.

IL-2 (PROLEUKIN (aldesleukin), Clinigen, 1.3 mg/vial, lyophilized) was reconstituted with 1.2 mL water to be at 1.1 mg/mL and mixed with abatacept (ORENCIA, Bristol Myers Squibb, 87.5 mg/0.7 mL, PFS (prefilled syringe) to obtain a combination formulation with a IL-2:abatacept mass ratio of 480:1 (101 mg/mL abatacept and 0.21 mg/mL IL-2). The solution was kept under STC with samples taken at start, 3 hours, 6 hours and 24 hours, and assessed by UV spectroscopy (A280/340), light obscuration (HIAC), and dynamic light scattering (DLS).

For UV spectroscopy, samples of combination formulation were diluted 135 times with saline, and absorbance measured on a Thermo Scientific Evolution 220 UV/Vis spectrophotometer (sample volume of 0.45 mL) at 280 nm and 340 nm. An aggregation index (AI) was determined where AI=100*(A340)/(A280−A340). Results are shown in Table 8, where changes in the concentration is within the method variability. These results show that the IL-2/abatacept solution concentration (mg/mL) is stable for 24 hours (the last timepoint tested) at room temperature.

TABLE 8
Absorbance of IL-2/Abatacept co-formulation

| | | | | | Ave. | Ave. |
|---|---|---|---|---|---|---|
| | | OD | % CV of | OD | Conc. | Aggregation |
| Time | Sample | @ 280 | OD280 | @ 340 | mg/mL | Index |
| T0 | 1 | 0.766 | 2% | 0.039 | 89.2 | 3.8 |
| | 2 | 0.762 | | 0.033 | | |
| | 3 | 0.742 | | 0.012 | | |
| T3 h | 1 | 0.822 | 2% | 0.056 | 93.9 | 5.4 |
| | 2 | 0.810 | | 0.043 | | |
| | 3 | 0.793 | | 0.026 | | |
| T6 h | 1 | 0.778 | 2% | 0.039 | 90.3 | 4.1 |
| | 2 | 0.772 | | 0.034 | | |
| | 3 | 0.753 | | 0.017 | | |
| T24 h | 1 | 0.771 | 1% | 0.026 | 91.4 | 3.4 |
| | 2 | 0.763 | | 0.017 | | |
| | 3 | 0.779 | | 0.032 | | |

Triplicate samples (0.05 mL), undiluted were added to wells of Thermo Scientific flat-bottom clear 96-well plate and light scattering measure at A280 and A340 in a BioTeck Synergy HTX Multi-mode Reader. Results are shown in Table 9. These results show that light scattering is stable for at least 24 hours (the final timepoint tested) at room temperature, indicating that the IL-2/abatacept combination formulation is stable and does not have a tendency to form aggregates and particles for at least 24 hours at room temperature.

TABLE 9
IL-2/abatacept co-formulation light scattering

| | Average | Average | Average |
|---|---|---|---|
| Time | 280 nm | 340 nm | AI |
| T0 | 3.301 | 0.178 | 5.7 |
| T3 h | 3.291 | 0.173 | 5.5 |
| T6 h | 3.284 | 0.167 | 5.4 |
| T24 h | 3.293 | 0.172 | 5.5 |

HIAC results of the samples from the BioTeck Synergy HTX Mulit-mode Reader for sub-visible particles is shown in Table 10. These results show that sub-visible particle counts for >2 μm and >5 μm are stable for at least 24 hours (the final timepoint tested), and that the sub-visible particle counts for >10 μm and >25 μm are stable for 24 hours and within the EU and USP requirements.

TABLE 10
IL-2/abatacept co-formulation - sub-visible particles.

| | |
|---|---|
| Sample (average | Particle Size (μm) |

| | | | | |
|---|---|---|---|---|
| of 3 | >2 | >5 | >10 | >25 |

| | |
|---|---|
| measurements) | Particles per mL |

| | | | | |
|---|---|---|---|---|
| Water Blank | 3.3 | 1.7 | 0.8 | 0.0 |
| Sample 1, T0 | 17353.3 | 4615.0 | 539.2 | 0.8 |
| Sample 2, T3 h | 19045.8 | 5508.3 | 685.0 | 5.8 |
| Sample 3, T6 h | 18584.2 | 5268.3 | 654.2 | 0.8 |
| Sample 4, T24 h | 18455.8 | 5105.0 | 665.0 | 4.2 |

DLS results are summarized in Table 11. The DLS results indicated that the size of particles in the IL-2/Abatacept combination formulation are stable for 24 hours. Most of the particles were in the main peak. The PDI is high. This might be because the particle size distribution beyond the main peak is diverse.

TABLE 11
IL-2/abatacept co-formulation - size of particles by DLS

| | Cumulant analysis | | Distribution analysis | |
|---|---|---|---|---|
| | Z-average, | | Size Main Peak, | |
| Sample Name | nm | PDI | nm | % Main Peak |
| Average T0 | 115.4 | 1.000 | 9.1 | 99.0 |
| Sample | | | | |
| Average T3 h | 143.8 | 1.000 | 9.4 | 98.9 |
| Sample | | | | |
| Average T6 h | 181.4 | 1.000 | 9.5 | 98.7 |
| Sample | | | | |
| Average T24 h | 277.8 | 0.967 | 9.0 | 99.1 |
| Sample | | | | |

### 6.6 Example 6: Phase I Trial Using Abatacept and Interleukin-2 (I1-2) in Patients with Amyotrophic Lateral Sclerosis (ALS)

The purpose of this study is to assess the effects of low dose abatacept followed by IL-2 administration in patients with ALS. In particular, this phase 1 study aims to determine whether the combination therapy of subcutaneous IL-2 and abatacept (Orencia®) is safe and well-tolerated in patients with ALS, and whether the therapy enhances Treg numbers and suppressive function in vivo.

6.6.1. Primary Objective

To assess the safety and the tolerability of abatacept followed by IL-2 administration in ALS patients, administered according to the dosage described in this protocol.

6.6.2. Secondary Objective

To investigate the immunomodulatory effects of abatacept followed by IL-2, administered in patients with ALS, by comparing before, during and after treatment: (a) the number of Tregs; (b) the suppressive activity of Tregs on T effector proliferation; (c) the level of cytokines secreted by PBMCs throughout the course of the study; and (d) the disease progression as determined by clinical outcome measures of ALS, including the Appel ALS Rating Scale (AALS) and ALS Functional Rating Scale-Revised (ALSFRS-R) scores, and the forced vital capacity (FVC) and maximum inspiratory pressure (MIP).

6.6.3. Study Design

This is a Phase I open-labeled uncontrolled study to assess the safety of the administration of abatacept followed by subcutaneous IL-2 administration. In addition to assessing the safety and toxicity of the low-dose abatacept followed by IL-2 administration in treating the progression of ALS, the goal of this phase I study is to evaluate the magnitude of Treg population enhancement in the subjects.

ALS patients receive a fixed low-dose of abatacept and IL-2 treatment for a total of 4 months. In particular, patients receive a fixed dose of subcutaneous abatacept (125 mg/mL) at day 1 of week 1. Two weeks later (Day 1 of week 3) patients receive a second dose of subcutaneous abatacept (125 mg/mL). In addition, patients receive subcutaneous IL-2 (1×10⁶ units/day) for 5 days (day 2-5 of week 3). If this treatment regimen is tolerated, patients receive 6 further similar treatment courses of abatacept and IL-2 every two weeks. See Table 12 below for administration schedule.

TABLE 12
Abatacept/IL-2 Administration Schedule

| Weeks of Therapy | | | | | | | | | | | | | | | |
|---|---|---|---|---|---|---|---|---|---|---|---|---|---|---|---|
| 1 | 2 | 3 | 4 | 5 | 6 | 7 | 8 | 9 | 10 | 11 | 12 | 13 | 14 | 15 | 16 |

| | | | | | | | | |
|---|---|---|---|---|---|---|---|---|
| Abatacept | D 1 | D 1 | D 1 | D 1 | D 1 | D 1 | D 1 | D 1 |
| 125 mg/ml | | | | | | | | |
| IL-2 | | D | D | D | D | D | D | D |
| Injection | | 1-5 | 1-5 | 1-5 | 1-5 | 1-5 | 1-5 | 1-5 |
| 10⁶ unit | | | | | | | | |

6.6.4. Drug Information

The recombinant human IL-2 used is PROLEUKIN (aldesleukin). Abatacept used is that in the ORENCIA product. See Section 6.2.4, above.

6.6.5. Patient Evaluation

6.6.5.1 Prior to Beginning Therapy

The following pre-treatment evaluation is completed prior to first abatacept/IL-2 injections: (a) Baseline history and physical exam; (b) Vital signs, Weight; (c) Pulse oximetry; (d) Safety labs: Complete blood count, PT/PTT, chemistries, liver function tests, thyroid function tests, QuantiFERON; (e) Baseline ECG; and (f) Research Labs: Immune biomarkers and Treg function and flow assays.

6.6.5.2 During Therapy

The following data is obtained on Day 1 and every two weeks while receiving abatacept/IL-2 infusions: Vital signs, Weight, Pulse oximetry.

The following lab tests are obtained every two weeks during abatacept/IL-2 therapy: Complete blood count, chemistries, liver function tests.

The following lab test are obtained twice (Day 1 and Day 8 of each treatment cycle) every two weeks during abatacept/IL-2 therapy: Immune biomarkers: Plasma, serum and messenger RNA.

The following lab test is obtained on days 1, 8, 22, 50, 78 and 106 during abatacept/IL-2 therapy: Treg function and flow assays.

6.6.5.3 Post Therapy

Once IL-2 treatment cycles are complete, the treated patient undergoes the following investigations at weeks 17 and 24: (a) Physical exam; (b) Vital signs, weight, and pulse oximetry; and (c) Labs: complete blood count, chemistries, liver function tests, thyroid function tests, Immune biomarkers and Treg function and flow assays (week 24 only).

6.6.5.4 Disease Specific Evaluation

Measurements of disease progression including the Appel ALS Rating Scale (AALS), ALS Functional Rating Scale-Revised (ALSFRS-R), Forced vital capacity (FVC) and Mean inspiratory pressure (MIP) is performed at screening, baseline (week 1), and weeks 5, 9, 13, 17 and 24 of the trial.

6.6.5.5 Immune Reconstitution Analysis

Depending on the availability of patient samples and reagents, immune reconstitution studies, including immunophenotyping and functional analysis, are obtained serially at the following time points before, during and after treatment.

Approximately 2-5 tablespoons (30-75 cc) of patient blood is taken, if feasible, at baseline visit and day 1 and day 8 of each treatment cycle during therapy (twice biweekly), then at weeks 17 and 24.

If a patient's hemoglobin is less than 8.0 g/dL at any of the evaluation times, the amount of blood drawn for evaluation is reduced and may be obtained over more than one venipuncture, if necessary.

Management of hypotension, management of respiratory distress/dyspnea, management of infection, and modifications of therapy are as described above in Example 2.

6.6.6. Inclusion Criteria for Enrollment

Subjects are eligible for initial enrollment on this study if they meet the following criteria:
(a) Provided informed consent and authorized use of protected health information (PHI) in accordance with national and local patient privacy regulations.
(b) ALS meeting El Escorial criteria for possible, probable, lab-supported probable, or definite ALS.
(c) At least 18 years old.
(d) Total bilirubin less than or equal to 1.5 mg/dL.
(e) Alanine aminotransferase level (ALT) less than or equal to five times normal, albumin greater than or equal to 3.0 gm/dL.
(f) Serum creatinine less than 1.5 mg/dL.
(g) Capable of complying with all study procedures, including the study drug delivery.
(h) A family member or caretaker who is expected to be consistently available to administer both study drugs of abatacept and IL-2 if the participant is unable to do so.
(i) On a stable regimen of riluzole for at least 30 days at the time of screening. If not on riluzole at the time of study entry, willing to refrain from initiation of the agent for the duration of the trial.
(j) Patients on edaravone willing to refrain from taking edaravone on the same day as they receive the abatacept injection for the duration of the trial. If not on edaravone at the time of study entry, willing to refrain from initiation of the agent for the duration of the trial.
(k) Forced vital capacity (FVC) >50% of predicted capacity for age, height, and sex at screening, or receiving treatment with noninvasive ventilation if FVC <50% of predicted for age, height and sex at screening.

6.6.7. Exclusion Criteria for Enrollment

Patients are ineligible to participate if any of the following are true at the time of screening:
(a) Serious, active bacterial, fungal or viral infection, active or latent tuberculosis.
(b) Tracheostomy.
(c) Severe cardiac dysfunction defined as left ventricular ejection fraction <40% if an echocardiogram is medically indicated to clarify ongoing symptoms or EKG findings; a history of non-controlled cardiac arrhythmias; history of cardiac tamponade; Unstable angina or MI in the last 3 months.
(d) Hypersensitivity or allergy to IL-2 or abatacept.
(e) History of bowel ischemia/perforation, or GI bleeding requiring surgery.
(f) History of resistant seizures, history of coma or toxic psychosis lasting >48 hours.
(g) Platelets <100,000/mm3; hematocrit <30%.
(h) History of cancer in the past 5 years (except cutaneous Basal cell carcinoma or squamous cell carcinoma).
(i) Hx of immunomodulation therapy including IL-2 or abatacept administration in the past 90 days.
(j) Treatment with another investigational drug, biological agent, or device within 30 days or 5 half-lives of screening, whichever is longer.
(k) If female, breastfeeding, known to be pregnant, planning to become pregnant during the study, or unwilling to use effective contraception for the duration of the trial and for 90 days after treatment.
(l) If male of reproductive capacity, unwilling to use effective contraception for the duration of the trial and for 90 days after treatment.

### 6.7 Example 7: Interim Results from Ongoing Phase I Trial Using Abatacept and Interleukin-2 (Il-2) in Patients with Amyotrophic Lateral Sclerosis (ALS)

The phase I trial described in Example 6 above involves abatacept and IL-2 treatment for a total of 4 months. To date, subjects enrolled in the trial have been on the treatment regimen at least 3 weeks, and the trial is ongoing. In this example, interim results (i.e., results available to date, since the trial is in progress) are provided from 4 ALS patients enrolled in the trial.

Table 13 below supplies the enrolled subjects' demographics and baseline characteristics.

TABLE 13
Enrolled Subjects' Demographics and Baseline Characteristics

| | Age | | | | Respiratory | Respiratory |
|---|---|---|---|---|---|---|
| | (years) | Sex | Type | Onset | Status | Support |
| Subject 1 | 47 | Female | Familial | Limb | No Respiratory | None |
| | | | | | Insufficiency | |
| Subject 2 | 54 | Male | Sporadic | Limb | Respiratory | Non-invasive |
| | | | | | Insufficiency | Ventilation |
| Subject 3 | 57 | Female | Sporadic | Bulbar | Respiratory | Non-invasive |
| | | | | | Insufficiency | Ventilation |
| Subject 4 | 84 | Female | Sporadic | Bulbar | Respiratory | None |
| | | | | | Insufficiency | |

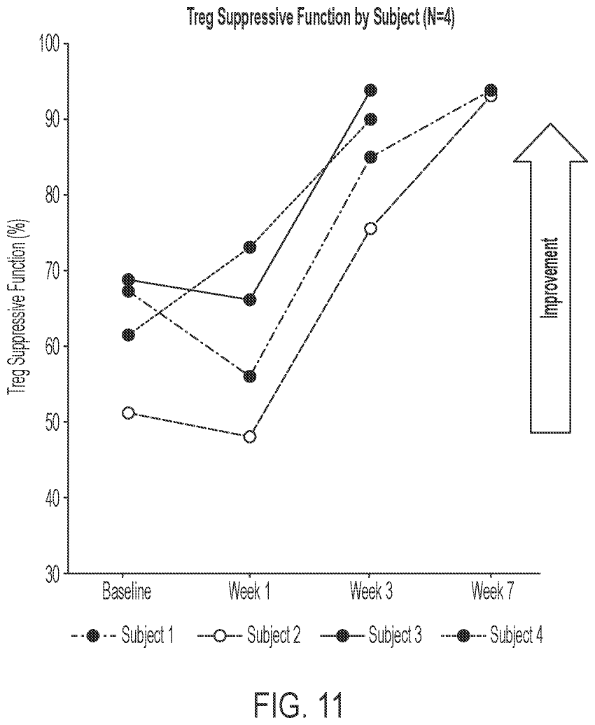

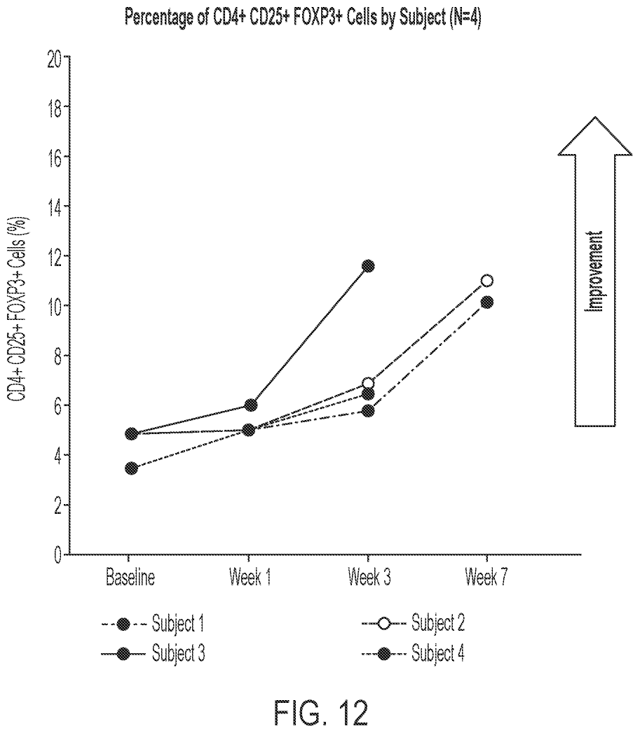

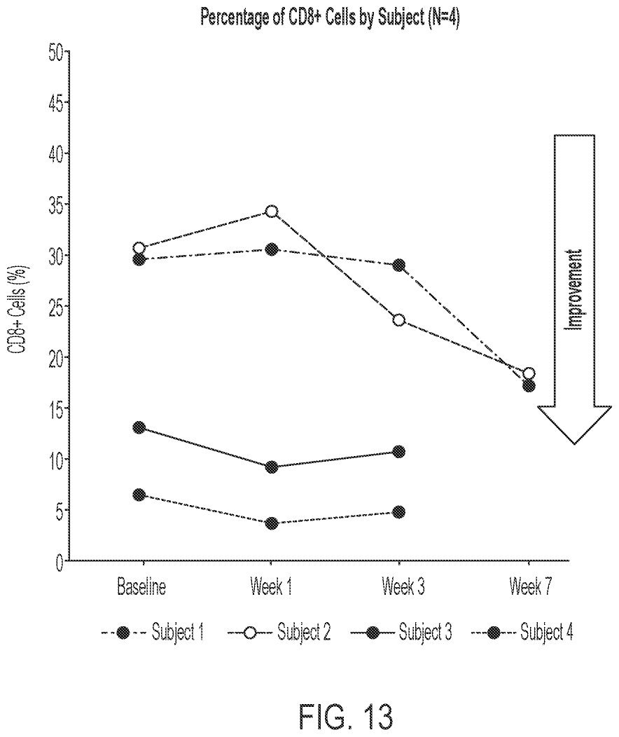

FIGS. 11-13 show Treg suppressive function (FIG. 11), CD4+CD25+ FOXP3+ Treg cell surface phenotype (FIG. 12) and CD8+ cell surface phenotype (FIG. 13) from each of these subjects. Abatacept alone was administered at day 1 immediately following baseline measurements. In FIGS. 11-13, “Week 1” refers to measurements taken the week following the abatacept only-administration. As explained above, abatacept/IL-2 combination treatment began two weeks after the abatacept-only administration and continued every two weeks thereafter during the course of the treatment. In FIGS. 11-13, “Week 3” refers to measurements taken the week following the first abatacept/IL-2 administration and “Week 7” refers to measurements taken the week following the third abatacept/IL-2 administration.

FIG. 11 shows the Treg suppressive function in Tregs from each of subjects. As the figure demonstrates, following the introduction of abatacept/IL-2 dual administration, improvement in Treg suppressive function in each subject relative to the respective baseline value was observed.

The percentage of cells expressing a CD4+CD25+ FOXP3+ Treg phenotype in the subjects is shown in FIG. 12. As the figure demonstrates, the percentage of cells exhibiting such a Treg phenotype has increased relative to baseline in each of the four subjects as the treatment regimen has progressed.

The percentage of Tregs expressing a CD8+ cytotoxic and pro-inflammatory phenotype is shown in FIG. 13. As the figure demonstrates, the percentage of cells exhibiting such a phenotype remained stable or improved (decreased) relative to baseline in each of the four subjects by week 3 (that is, the week following the first abatacept/IL-2 administration). Interestingly, an improvement (decrease) in the percentage of CD8+ cells has been observed in both subjects (Subjects 2 and 4) that have progressed to week 7 (the week following the third abatacept/IL-2 administration) of the treatment regimen.

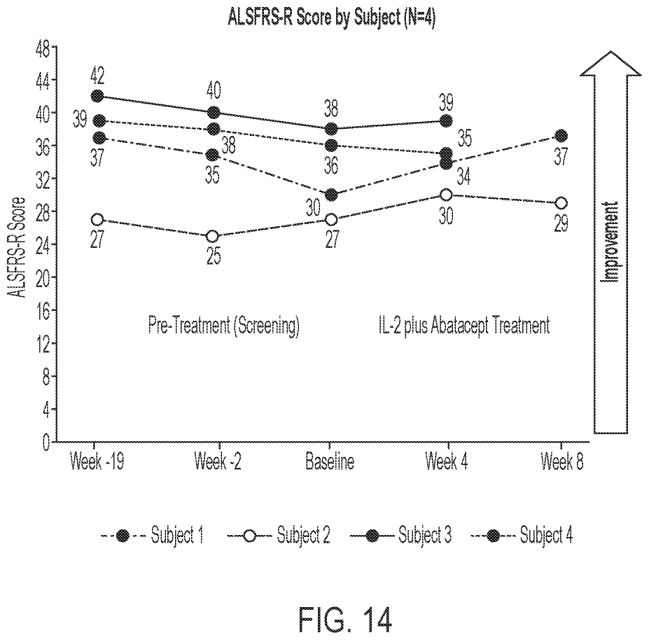

Disease progression has been monitored by the ALSFRS-R score in the 4 subjects receiving abatacept/IL-2 treatment (FIG. 14). The ALSFRS-R is a widely accepted and validated outcome measure of activity limitation for patients with ALS. It comprises 12 items rated on a scale of 0 to 4 (4=normal function; 0=complete loss of function). The 12 items are grouped into 4 domains: bulbar, fine motor, gross motor, and respiratory. Each point decrease on the ALSFRS-R represents lost capability in performing activities fundamental to daily life. ALS progression is therefore reflected in lowering ALSFRS-R score over time.

As shown in FIG. 14, each subject progressed at a varying rate prior to enrollment in the study (“Pre-Treatment (Screening)”). Once enrolled, the ALSFRS-R was measured at baseline (immediately prior to administration of the abatacept-only dose) and then has been measured every 4 weeks during the study (“Week 4” and “Week 8”). As shown in FIG. 14, overall, disease progression as assessed by ALSFRS-R scoring, has been stable in the four subjects since the initiation of abatacept/IL-2 treatment.

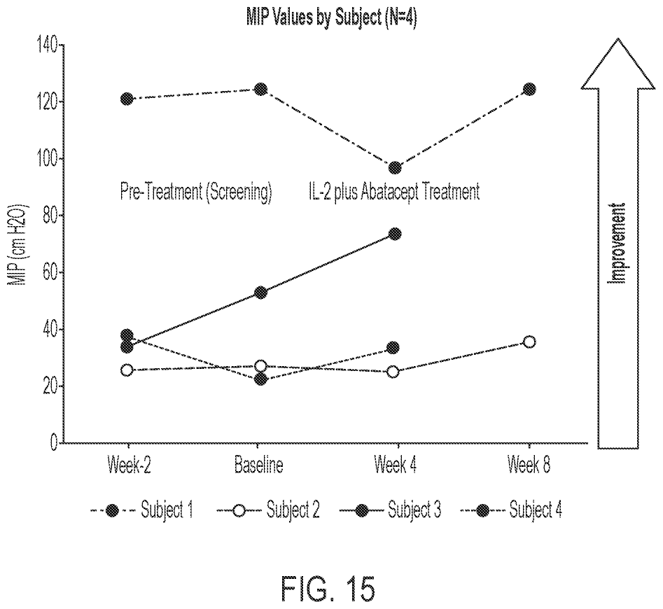

Disease progression has also been monitored by the maximal inspiratory pressure (MIP) in the 4 subjects receiving abatacept/IL-2 treatment (FIG. 15). Maximal inspiratory pressure (MIP) is a measure of the strength of inspiratory muscles, primarily the diaphragm, and allows for the assessment of ventilatory failure, restrictive lung disease and respiratory muscle strength. A reduction in MIP is associated with a progressive clinical worsening in patients with various conditions, including ALS. FIG. 15 show MIP values in in the 4 subjects enrolled in the phase I trial prior to and after onset of treatment with IL-2 and abatacept. As shown in FIG. 15, overall, disease progression, as assessed by MIP, has been stable in the four subjects since the initiation of abatacept/IL-2 treatment.

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method of treating a neurodegenerative or neuroinflammatory disease or disorder in a subject in need thereof, comprising administering to the subject:
i) a CTLA-4-containing protein; and
ii) an IL-2 protein;
wherein the method mitigates one or more symptoms associated with the neurodegenerative or neuroinflammatory disease or disorder in the treated subject.

2. The method of claim 1, wherein the CTLA-4-containing protein comprises a human CTLA-4 extracellular domain.

3. The method of claim 1 or 2, wherein the CTLA-4-containing protein is a fusion protein.

4. The method of claim 3, wherein the fusion protein comprises a human CTLA-4 extracellular domain and a human immunoglobulin Fc domain.

5. The method of claim 4, wherein the Fc domain is a modified Fc domain that comprises an immunoglobulin hinge region, CH2 region and CH3.

6. The method of claim 4 or 5, wherein the human immunoglobulin Fc domain is a human IgG1 Fc domain.

7. The method of any one of claims 1-6, wherein the CTLA-4-containing protein is glycosylated.

8. The method of any one of claims 1-7, wherein the CTLA-4-containing protein the following amino acid sequence monomer:
(SEQ ID NO: 1)
MHVAQPAVVLASSRGIASFVCEYASPGKATEVRVTVLRQADSQVTEVCA
ATYMMGNELTFLDDSICTGTSSGNQVNLTIQGLRAMDTGLYICKVELMY
PPPYYLGIGNGTQIYVIDPEPCPDSDQEPKSSDKTHTSPPSPAPELLGG
SSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGK.

9. The method of claim 8, wherein the CTLA-4-containing protein comprises a homodimer of two monomers, each comprising the amino acid sequence of SEQ ID NO:1.

10. The method of claim 1, wherein the CTLA-4-containing protein is abatacept.

11. The method of any one of claims 1-10, wherein the IL-2 protein is a human IL-2 protein.

12. The method of claim 11, wherein the human IL-2 protein comprises a serine at the amino acid position corresponding to native mature human IL-2 amino acid residue 125.

13. The method of claim 11 or 12, wherein the human IL-2 protein lacks an N-terminal alanine amino acid.

14. The method of any one of claims 11-13, wherein the human IL-2 protein comprises the following amino acid sequence:
(SEQ ID NO: 3)
PTSSSTKKTQLQLEHLLLDLQMILNGINNYKNPKLTRMLTFKFYMPKKA
TELKHLQLEEELKPLEEVLNLAQSKNFHLRPRDLISNINVIVLELKGSE
TTFMCEYADETATIVEFLNRWITFSQSIISTLT.

15. The method of any one of claims 1-14, wherein the IL-2 protein is not glycosylated.

16. The method of claim 11, wherein the IL-2 protein is aldesleukin.

17. A method of treating a neurodegenerative or neuroinflammatory disease or disorder in a subject in need thereof, comprising administering to the subject:
i) abatacept; and
ii) aldesleukin;
wherein the method mitigates one or more symptoms associated with the neurodegenerative or neuroinflammatory disease or disorder in the treated subject.

18. The method of claim 17, wherein the abatacept is administered by injection or infusion.

19. The method of 18, wherein the abatacept is administered subcutaneously.

20. The method of 18, wherein the abatacept is administered intravenously.

21. The method of claim 17, wherein the aldesleukin is administered by injection or infusion.

22. The method of 21, wherein the aldesleukin is administered subcutaneously.

23. The method of 21, wherein the aldesleukin is administered intravenously.

24. The method of claim 17, wherein the abatacept and the aldesleukin are administered subcutaneously.

25. The method of claim 17, wherein the abatacept and the aldesleukin are administered intravenously.

26. The method of any one of claims 17-25, wherein the abatacept is administered once every two weeks.

27. The method of claim 26, wherein the abatacept is administered subcutaneously once every two weeks.

28. The method of claim 26 or 27, wherein the abatacept is administered once every two weeks for 15 weeks.

29. The method of any one of claims 17-28, wherein the aldesleukin is administered once daily for three consecutive days.

30. The method of claim 29, wherein the aldesleukin is administered subcutaneously once daily for three consecutive days.

31. The method of any one of claims 17-25, wherein:
a) the abatacept is administered once every two weeks; and
b) the aldesleukin is administered once daily for three consecutive days beginning on the day the abatacept is administered.

32. The method of claim 31, wherein the abatacept and the aldesleukin are administered subcutaneously.

33. The method of any one of claims 17-25, wherein:
a) the abatacept is administered once every two weeks for fifteen weeks;
b) aldesleukin administration begins on week three; and
c) once aldesleukin administration begins, the aldesleukin is administered once daily for three consecutive days beginning on the day the abatacept is administered.

34. The method of claim 33, wherein the abatacept and the aldesleukin are administered subcutaneously.

35. The method of any one of claims 17-34, wherein the abatacept is administered in an amount in the range of 50 mg to 125 mg.

36. The method of claim 35, wherein the abatacept is administered in a 50 mg amount.

37. The method of claim 36, wherein the abatacept is subcutaneously administered in a 0.4 mL volume.

38. The method of claim 35, wherein the abatacept is administered in an 87.5 mg amount.

39. The method of claim 38, wherein the abatacept is subcutaneously administered in a 0.7 mL volume.

40. The method of claim 35, wherein the abatacept is administered in a 125 mg amount.

41. The method of claim 40, wherein the abatacept is subcutaneously administered in a 1.0 mL volume.

42. The method of any one of claims 17-41, wherein the aldesleukin is administered in an amount in the range of 500,000 units to 3,000,000 units.

43. The method of claim 42, wherein the aldesleukin is administered in an amount in the range of 500,000 units to 2,000,000 units.

44. The method of claim 43, wherein the aldesleukin is administered in an amount of 1,000,000 units.

45. The method of any one of claims 42-44, wherein the aldesleukin is administered subcutaneously.

46. A method of treating a neurodegenerative or neuroinflammatory disease or disorder in a subject in need thereof, comprising a dosing cycle that begins on day 1 and comprises administering to the subject a formulation comprising:
i) abatacept; and
ii) aldesleukin;
wherein the method mitigates one or more symptoms associated with the neurodegenerative or neuroinflammatory disease or disorder in the treated subject.

47. The method of claim 46, wherein the formulation is administered by injection or infusion.

48. The method of 46, wherein the formulation is administered subcutaneously.

49. The method of 46, wherein the formulation is administered intravenously.

50. The method of any one of claims 46-49, wherein the dosing cycle comprises administering the formulation to the subject 1-10 times.

51. The method of any one of claims 46-49, wherein the dosing cycle comprises a single administration of the formulation to the subject on day 1 of the dosing cycle.

52. The method of any one of claims 46-49, wherein the dosing cycle comprises administering the formulation to the subject daily for two consecutive days, beginning on day 1 of the dosing cycle.

53. The method of any one of claims 46-49, wherein the dosing cycle comprises administering the formulation to the subject daily for three consecutive days, beginning on day 1 of the dosing cycle.

54. The method of any one of claims 46-49, wherein the dosing cycle comprises administering the formulation to the subject daily for four consecutive days, beginning on day 1 of the dosing cycle.

55. The method of any one of claims 46-49, wherein the dosing cycle comprises administering the formulation to the subject daily for five consecutive days, beginning on day 1 of the dosing cycle.

56. The method of any one of claims 46-55, wherein the dosing cycle is repeated 1-12 times.

57. The method of any one of claims 46-55, wherein the dosing cycle is repeated 6 times.

58. The method of claim 56 or 57, wherein each repeated dosing cycle begins 10-28 days after day 1 of the previous dosing cycle.

59. The method of any one of claims 56-58, wherein each repeated dosing cycle begins 14 days after day 1 of the previous dosing cycle.

60. The method of any one of claims 46-49, wherein the first dosing cycle comprises administering the formulation to the subject daily for three consecutive days, beginning on day 1 of the dosing cycle, and the first dosing cycle is repeated 6 times, with each repeated dosing cycle beginning 14 days after day 1 of the previous dosing cycle.

61. The method of any one of claims 46-49, wherein the dosing cycle comprises administering to the subject a formulation comprising about 5 mg to about 125 mg abatacept and about 3×10⁴ to about 3×10⁷ units aldesleukin.

62. The method of any one of claims 46-49, wherein the dosing cycle comprises administering to the subject a formulation comprising about 8.75 mg to about 87.5 mg abatacept and about 3×10⁴ to about 3×10⁷ units aldesleukin.

63. The method of any one of claims 46-49, wherein the dosing cycle comprises administering to the subject a formulation comprising about 29.17 mg abatacept and about 1×10⁵ units aldesleukin.

64. The method of any one of claims 46-49, wherein the dosing cycle comprises administering to the subject a formulation comprising about 29.17 mg abatacept and about 1×10⁶ units aldesleukin.

65. The method of any one of claims 46-49, wherein the dosing cycle comprises administering to the subject a formulation comprising about 29.17 mg abatacept and about 1×10⁷ units aldesleukin.

66. The method of any one of claims 46-49, wherein the dosing cycle comprises administering to the subject a formulation comprising about 5 mg to about 50 mg abatacept and about 3×10⁴ to about 3×10⁷ units aldesleukin.

67. The method of any one of claims 46-49, wherein the dosing cycle comprises administering to the subject a formulation comprising about 16.67 mg abatacept and about 1×10⁵ units aldesleukin.

68. The method of any one of claims 46-49, wherein the dosing cycle comprises administering to the subject a formulation comprising about 16.67 mg abatacept and about 1×10⁶ units aldesleukin.

69. The method of any one of claims 46-49, wherein the dosing cycle comprises administering to the subject a formulation comprising about 16.67 mg abatacept and about 1×10⁷ units aldesleukin.

70. The method of any one of claims 46-49, wherein the dosing cycle comprises administering to the subject a formulation comprising about 12.5 mg to about 125 mg abatacept and about 3×10⁴ to about 3×10⁷ units aldesleukin.

71. The method of any one of claims 46-49, wherein the dosing cycle comprises administering to the subject a formulation comprising about 41.67 mg abatacept and about 1×10⁵ units aldesleukin.

72. The method of any one of claims 46-49, wherein the dosing cycle comprises administering to the subject a formulation comprising about 41.67 mg abatacept and about 1×10⁶ units aldesleukin.

73. The method of any one of claims 46-49, wherein the dosing cycle comprises administering to the subject a formulation comprising about 41.67 mg abatacept and about 1×10⁷ units aldesleukin.

74. The method of any one of claims 46-49, wherein the dosing cycle comprises administering the formulation to the subject daily for three consecutive days, beginning on day 1 of the dosing cycle, wherein the formulation comprises about 29.17 mg abatacept and about 1×10⁶ units aldesleukin.

75. The method of any one of claims 46-49, wherein a total of 50 mg abatacept and 3×10⁵ units aldesleukin are administered to the subject per dosing cycle.

76. The method of claim 75, wherein the dosing cycle comprises 1-10 administrations of an abatacept/aldesleukin formulation as shown at Table 1A.

77. The method of any one of claims 46-49, wherein a total of 50 mg abatacept and 3×10⁶ units aldesleukin are administered to the subject per dosing cycle.

78. The method of claim 77, wherein the dosing cycle comprises 1-10 administrations of an abatacept/aldesleukin formulation as shown at Table 1B.

79. The method of any one of claims 46-49, wherein a total of 50 mg abatacept and 3×10⁷ units aldesleukin are administered to the subject per dosing cycle.

80. The method of claim 79, wherein the dosing cycle comprises 1-10 administrations of an abatacept/aldesleukin formulation as shown at Table 1C.

81. The method of any one of claims 46-49, wherein a total of 87.5 mg abatacept and 3×10⁵ units aldesleukin are administered to the subject per dosing cycle.

82. The method of claim 81, wherein the dosing cycle comprises 1-10 administrations of an abatacept/aldesleukin formulation as shown at Table 2A.

83. The method of any one of claims 46-49, wherein a total of 87.5 mg abatacept and 3×10⁶ units aldesleukin are administered to the subject per dosing cycle.

84. The method of claim 83, wherein the dosing cycle comprises 1-10 administrations of an abatacept/aldesleukin formulation as shown at Table 2B.

85. The method of any one of claims 46-49, wherein a total of 87.5 mg abatacept and 3×10⁷ units aldesleukin are administered to the subject per dosing cycle.

86. The method of claim 85, wherein the dosing cycle comprises 1-10 administrations of an abatacept/aldesleukin formulation as shown at Table 2C.

87. The method of any one of claims 46-49, wherein a total of 125 mg abatacept and 3×10⁵ units aldesleukin are administered to the subject per dosing cycle.

88. The method of claim 87, wherein the dosing cycle comprises 1-10 administrations of an abatacept/aldesleukin formulation as shown at Table 3A.

89. The method of any one of claims 46-49, wherein a total of 125 mg abatacept and 3×10⁶ units aldesleukin are administered to the subject per dosing cycle.

90. The method of claim 89, wherein the dosing cycle comprises 1-10 administrations of an abatacept/aldesleukin formulation as shown at Table 3B.

91. The method of any one of claims 46-49, wherein a total of 125 mg abatacept and 3×10⁷ units aldesleukin are administered to the subject per dosing cycle.

92. The method of claim 91, wherein the dosing cycle comprises 1-10 administrations of an abatacept/aldesleukin formulation as shown at Table 3C.

93. The method of any one of claims 46-92, wherein the dosing cycle is repeated 6 times, with each repeated dosing cycle beginning 14 days after day 1 of the previous dosing cycle.

94. The method of any one of claims 46-92, wherein the formulation is administered by injection or infusion.

95. The method of any one of claims 46-92, wherein the formulation is administered subcutaneously.

96. The method of any one of claims 46-92, wherein the formulation is administered intravenously.

97. The method of any one of claims 46-96, further comprising administering an abatacept formulation to the subject 14 days prior to day 1 of the first dosing cycle, wherein the abatacept formulation comprises abatacept.

98. The method of claim 97, wherein the abatacept formulation comprises 50 mg to 125 mg abatacept.

99. The method of claim 97, wherein the abatacept formulation comprises 87.5 mg abatacept.

100. The method of any one of claims 97-99, wherein the abatacept formulation is administered by injection or infusion.

101. The method of any one of claims 97-99, wherein the abatacept formulation is administered subcutaneously.

102. The method of any one of claims 97-99, wherein the abatacept formulation is administered intravenously.

103. The method of any one of claims 97-99, wherein the neurodegenerative disease or disorder is amyotrophic lateral sclerosis, Alzheimer's disease, Parkinson's disease, multiple sclerosis, frontotemporal dementia or Huntington's disease.

104. The method of claim 103, wherein the neurodegenerative disease or disorder is Alzheimer's disease.

105. The method of any one of claims 1-104, wherein the neuroinflammatory disease or disorder is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

106. The method of any one of claims 1-105, wherein the method further comprises performing an additional therapeutic intervention comprising a cognitive rehabilitation program, a neurostimulation technique, or a combination thereof.

107. The method of claim 106, wherein the cognitive rehabilitation program is a computer-implemented cognitive rehabilitation program.

108. The method of claim 105 or 106, wherein the neurostimulation technique is an invasive brain stimulation (IBS) technique.

109. The method of claim 105 or 106, wherein the neurostimulation technique is a non-invasive brain stimulation (NIBS) technique.

110. The method of claim 108, wherein the IBS technique is selected from the group consisting of: deep brain stimulation (DBS) and invasive vagus nerve stimulation (VNS).

111. The method of claim 109, wherein the NIBS technique is selected from the group consisting of transcranial magnetic stimulation (TMS), transcranial direct current stimulation (tDCS), transcranial alternating current stimulation (tACS), electroconvulsive treatment (ECT), magnetic seizure therapy (MST), cranial electrostimulation (CES), and non-invasive VNS.

112. A kit, comprising, in separate containers, i) one or more doses of a formulation comprising 50 to 125 mg abatacept, and ii) one or more doses of a formulation comprising 500,000 to 3,000,000 units aldesleukin.

113. The kit of claim 112, wherein the kit comprises one or more doses of a formulation comprising 50 mg abatacept.

114. The kit of claim 112, wherein the kit comprises one or more doses of a formulation comprising 87.5 mg abatacept.

115. The kit of claim 112, wherein the kit comprises one or more doses of a formulation comprising 125 mg abatacept.

116. The kit of any one of claims 112-115, wherein the kit comprises one or more doses of a formulation of 500,000 to 2,000,000 units aldesleukin.

117. The kit of any one of claims 112-116, wherein the kit comprises one or more doses of a formulation of 1,000,000 units aldesleukin.

118. The kit of any one of claims 112-117, wherein the one or more doses of abatacept are present in lyophilized form.

119. The kit of claim 118, wherein the one or more doses of abatacept are present as a lyophilized powder or lyophilized cake.

120. The kit of any one of claims 112-119, wherein the one or more doses of aldesleukin are present in lyophilized form.

121. The kit of claim 120, wherein the one or more doses of aldesleukin are present as a lyophilized powder or lyophilized cake.

122. The kit of any one of claims 112-121, wherein the formulation of one or more doses of abatacept is suitable for subcutaneous administration.

123. The kit of any one of claims 112-121, wherein the formulation of one or more doses of abatacept is suitable for intravenous administration.

124. The kit of any one of claims 112-123, wherein the formulation of one or more doses of aldesleukin is suitable for subcutaneous administration.

125. The kit of any one of claims 112-123, wherein the formulation of one or more doses of aldesleukin is suitable for intravenous administration.

126. A pharmaceutical composition comprising one or more abatacept/aldesleukin doses.

127. The pharmaceutical composition of claim 126, wherein an abatacept/aldesleukin dose comprises 5 mg to 125 mg abatacept and 3×10⁴ to 3×10⁷ units aldesleukin.

128. The pharmaceutical composition of claim 126, wherein an abatacept/aldesleukin dose comprises 8.75 mg to about 87.5 mg abatacept and 3×10⁴ to 3×10⁷ units aldesleukin.

129. The pharmaceutical composition of claim 126, wherein an abatacept/aldesleukin dose comprises about 29.17 mg abatacept and about 1×10⁵ units aldesleukin.

130. The pharmaceutical composition of claim 126, wherein an abatacept/aldesleukin dose comprises about 29.17 mg abatacept and about 1×10⁶ units aldesleukin.

131. The pharmaceutical composition of claim 126, wherein an abatacept/aldesleukin dose comprises about 29.17 mg abatacept and about 1×10⁷ units aldesleukin.

132. The pharmaceutical composition of claim 126, wherein an abatacept/aldesleukin dose comprises about 5 mg to about 50 mg abatacept and about 3×10⁴ to about 3×10⁷ units aldesleukin.

133. The pharmaceutical composition of claim 126, wherein an abatacept/aldesleukin dose comprises about 16.67 mg abatacept and about 1×10⁵ units aldesleukin.

134. The pharmaceutical composition of claim 126, wherein an abatacept/aldesleukin dose comprises about 16.67 mg abatacept and about 1×10⁶ units aldesleukin.

135. The pharmaceutical composition of claim 126, wherein an abatacept/aldesleukin dose comprises about 16.67 mg abatacept and about 1×10⁷ units aldesleukin.

136. The pharmaceutical composition of claim 126, wherein an abatacept/aldesleukin dose comprises about 12.5 mg to about 125 mg abatacept and about 3×10⁴ to about 3×10⁷ units aldesleukin.

137. The pharmaceutical composition of claim 126, wherein an abatacept/aldesleukin dose comprises about 41.67 mg abatacept and about 1×10⁵ units aldesleukin.

138. The pharmaceutical composition of claim 126, wherein an abatacept/aldesleukin dose comprises about 41.67 mg abatacept and about 1×10⁶ units aldesleukin.

139. The pharmaceutical composition of claim 126, wherein an abatacept/aldesleukin dose comprises comprising about 41.67 mg abatacept and about 1×10⁷ units aldesleukin.

140. The pharmaceutical composition of claim 126, wherein the pharmaceutical compositions comprises one or more abatacept/aldesleukin doses as shown at Table 4.

141. The pharmaceutical composition of any one of claims 126-140, wherein the pharmaceutical composition is present in lyophilized form.

142. The pharmaceutical composition of claim 141, wherein the pharmaceutical composition is present as a lyophilized powder or lyophilized cake.

143. The pharmaceutical composition of any one of claims 126-140, wherein the pharmaceutical composition is a solution.

144. The pharmaceutical composition of claim 143, wherein the pharmaceutical composition is present as an aqueous solution.

145. The pharmaceutical composition of claim 143 or 144, wherein the one or more abatacept/aldesleukin doses are present at a concentration of 1 abatacept/aldesleukin dose/0.4 ml.

146. The pharmaceutical composition of claim 143 or 144, wherein the one or more abatacept/aldesleukin doses are present at a concentration of 1 abatacept/aldesleukin dose/0.7 ml.

147. The pharmaceutical composition of claim 143 or 144, wherein the one or more abatacept/aldesleukin doses are present at a concentration of 1 abatacept/aldesleukin dose/1.0 ml.

148. The pharmaceutical composition of claim 143 or 144, wherein the one or more abatacept/aldesleukin doses are present at a concentration of 1 abatacept/aldesleukin dose/1.5 ml.

149. The pharmaceutical composition of claim 143 or 144, wherein the one or more abatacept/aldesleukin doses are present at a concentration of 1 abatacept/aldesleukin dose/2.0 ml.

150. The pharmaceutical composition of any one of claims 126-149, wherein the pharmaceutical composition is suitable for subcutaneous administration.

151. The pharmaceutical composition of any one of claims 126-149, wherein the pharmaceutical composition is suitable for intravenous administration.
